SEQUENCE LISTING (48 sequences)

SEQ ID NO: 1 - protein, 11 aa, Flavivirus FSME
VHTWTEQYFKQ

SEQ ID NO: 2 - protein, 16 aa, Flavivirus FSME
GIRSTTRLENVMWKQI

SEQ ID NO: 3 - protein, 17 aa, Flavivirus FSME
EVEDYGFGVFTTNIWLK

SEQ ID NO: 4 - protein, 9 aa, Flavivirus FSME
RLMSAAIKD

SEQ ID NO: 5 - protein, 12 aa, Flavivirus FSME
AVHADMGYWIES

SEQ ID NO: 6 - protein, 15 aa, Flavivirus FSME
WPKSHTLWSNGVLES

SEQ ID NO: 7 - protein, 13 aa, Flavivirus FSME
RGPSLRTTTVSGK

SEQ ID NO: 8 - protein, 13 aa, Flavivirus FSME
GEDGCWYGMEIRP

SEQ ID NO: 9 - protein, 12 aa, Flavivirus FSME
GPWHLGKLELDF

SEQ ID NO: 10 - protein, 10 aa, Artificial Sequence
  DENV NS1
KYSWSEWGAK

SEQ ID NO: 11 - protein, 11 aa, Flavivirus FSME
KYSWKSWGKAK

SEQ ID NO: 12 - skipped

SEQ ID NO: 13 - protein, 4 aa, Flavivirus FSME
GGGC

SEQ ID NO: 14 - protein, 15 aa, Flavivirus FSME
DSGCVVSWKNKELKC

SEQ ID NO: 15 - protein, 15 aa, Flavivirus FSME
ITDNVHTWTEQYKFQ

SEQ ID NO: 16 - protein, 15 aa, Flavivirus FSME
TRLENLMWKQITPEL

SEQ ID NO: 17 - protein, 15 aa, Flavivirus FSME
ITPELNHILSENEVK

SEQ ID NO: 18 - protein, 15 aa, Flavivirus FSME
AECPNTNRAWNSLEV

SEQ ID NO: 19 - protein, 15 aa, Flavivirus FSME
MIIPKNLAGPVSQHN

SEQ ID NO: 20 - protein, 15 aa, Flavivirus FSME
VSQHNYRPGYHTQIT

SEQ ID NO: 21 - protein, 15 aa, Flavivirus FSME
TTASGKLITEWCCRS

SEQ ID NO: 22 - protein, 15 aa, Flavivirus FSME
LRYRGEDGCWYGMEI

SEQ ID NO: 23 - protein, 15 aa, Flavivirus FSME
KELKCGSGIFITDNV

SEQ ID NO: 24 - protein, 15 aa, Flavivirus FSME
QYKFQPESPSKLASA

SEQ ID NO: 25 - protein, 15 aa, Flavivirus FSME
KLASAIQKAHEEGIC

SEQ ID NO: 26 - protein, 15 aa, Flavivirus FSME
EEGICGIRSVTRLEN

SEQ ID NO: 27 - protein, 15 aa, Flavivirus FSME
ENEVKLTIMTGDIKG

SEQ ID NO: 28 - protein, 15 aa, Flavivirus FSME
GDIKGIMQAGKRSLR

SEQ ID NO: 29 - protein, 15 aa, Flavivirus FSME
KRSLRPQPTELKYSW

SEQ ID NO: 30 - protein, 15 aa, Flavivirus FSME
LKYSWKTWGKAKMLS

SEQ ID NO: 31 - protein, 15 aa, Flavivirus FSME
AKMLSTESHNQTFLI

SEQ ID NO: 32 - protein, 15 aa, Flavivirus FSME
QTFLIDGPETAECPN

SEQ ID NO: 33 - protein, 15 aa, Flavivirus FSME
NSLEVEDYGFGVFTT

SEQ ID NO: 34 - protein, 15 aa, Flavivirus FSME
GVFTTNIWLKLKEKQ

SEQ ID NO: 35 - protein, 15 aa, Flavivirus FSME
LKEKQDVFCDSKLMS

SEQ ID NO: 36 - protein, 15 aa, Flavivirus FSME
SKLMSAAIKDNRAVH

SEQ ID NO: 37 - protein, 15 aa, Flavivirus FSME
NRAVHADMGYWIESA

SEQ ID NO: 38 - protein, 15 aa, Flavivirus FSME
WIESALNDTWKIEKA

SEQ ID NO: 39 - protein, 15 aa, Flavivirus FSME
KIEKASFIEVKNCHW

SEQ ID NO: 40 - protein, 15 aa, Flavivirus FSME
KNCHWPKSHTLWSNG

SEQ ID NO: 41 - protein, 15 aa, Flavivirus FSME
LWSNGVLESEMIIPK

SEQ ID NO: 42 - protein, 15 aa, Flavivirus FSME
HTQITGPWHLGKLEM

SEQ ID NO: 43 - protein, 15 aa, Flavivirus FSME
GKLEMDFDFCDGTTV

SEQ ID NO: 44 - protein, 15 aa, Flavivirus FSME
DGTTVVVTEDCGNRG

SEQ ID NO: 45 - protein, 15 aa, Flavivirus FSME
CGNRGPSLRTTTASG

SEQ ID NO: 46 - protein, 15 aa, Flavivirus FSME
WCCRSCTLPPLRYRG

SEQ ID NO: 47 - protein, 15 aa, Flavivirus FSME
YGMEIRPLKEKEENL

SEQ ID NO: 48 - protein, 15 aa, Flavivirus FSME
LKEKEENLVNSLVTA

(19) United States
(12) Patent Application Publication
(10) Pub. No.: US 2024/0316174 A1
(43) Pub. Date: Sep. 26, 2024

(54) BACTERIOPHAGE VIRUS-LIKE PARTICLE VACCINES AGAINST FLAVIVIRUS NON-STRUCTURAL PROTEIN 1

(71) Applicant: UNM RAINFOREST INNOVATIONS, Albuquerque, NM (US)

(72) Inventors: KATHRYN MARIE FRIETZE, ALBUQUERQUE, NM (US); NICOLE WARNER, ALBUQUERQUE, NM (US)

(21) Appl. No.: 18/269,796

(22) PCT Filed: Jan. 27, 2022
(86) PCT No.: PCT/US22/14080

§ 371 Date: Jun. 27, 2023

Related U.S. Application Data
(60) Provisional application No. 63/253,205, filed on Oct. 7, 2021.
(60) Provisional application No. 63/146,347, filed on Feb. 5, 2021.

(51) Int. Cl. A61K 39/12; A61K 47/64; A61P 31/14; C07K 14/005; C12N 7/00

(52) U.S. Cl. CPC A61K 39/12; A61K 47/64; A61P 31/14; C07K 14/005; C12N 7/00; C12N 2770/24022; C12N 2770/24023; C12N 2770/24033; C12N 2795/18122; C12N 2795/18123

(57) ABSTRACT

The present invention is directed to virus-like particles (VLPs) which display immunogenic peptides of Flavivirus non-structural Protein 1 (NS1) derived from Dengue Virus (DENY), immunogenic compositions and vaccines against Flavivirus infection and related methods of immunizing and/or vaccinating subjects against Flavivirus, especially Dengue infections. The VLPs according to the present invention comprise polypeptide subunits of Dengue NS 1 protein which has been conjugated to the surface of a VLP as described herein, often a VLP derived from a Qbeta (Pβ) or AP205 bacteriophage.

### RELATED APPLICATIONS AND GRANT SUPPORT

This application claims the benefit of priority of US provisional applications s.n. U.S. 63/146,347, filed Feb. 5, 2021 and s.n. U.S. 63/253,205, filed Oct. 7, 2021 of identical title, the entire contents of each application being incorporated by reference herein.

This invention was made with government support under grant nos. K21 GM088021, R21 AI148835 and T32 AI0075238 awarded by the National Institute of Allergy & Infectious Diseases, National Institutes of Health and grant nos. ULITR001449 and KL2 TR001448 awarded by the National Center for Advancing Translational Sciences, National Institutes of Health. The government has certain rights in the invention.

### FIELD OF THE INVENTION

The present invention is directed to virus-like particles (VLPs) which display immunogenic peptides of Flavivirus non-structural Protein 1 (NS1) derived from Dengue Virus (DENV), immunogenic compositions and vaccines against Flavivirus infection, especially DENV and related methods of immunizing and/or vaccinating subjects against DENV infections. The VLPs according to the present invention comprise polypeptide subunits of Dengue NS1 protein, especially those that are conserved among the DENV serotypes, which are conjugated to the surface of a VLP as described herein, often a VLP derived from a Qbeta (Qβ) or AP205 bacteriophage.

### BACKGROUND AND OVERVIEW OF THE INVENTION

Dengue virus (DENV) is a mosquito-borne virus that infects over 390 million people worldwide annually, primarily in developing nations. There are currently no antiviral treatments for DENV and prevention efforts rely on local control of the vector Aedes aegypti mosquito populations. Although efforts to develop a DENV vaccine have been pursued for almost 90 years, a safe and effective vaccine remains elusive largely due to the unique pathogenic features of DENV infection. Recent concerns regarding the safety of Dengvaxia (Sanofi Pasteur) has highlighted the need for novel vaccine strategies for DENV. Recent research from several groups has implicated non-structural protein 1 (NS1) in the development of vascular leakage and progression to severe disease. NS1 is produced early in infection and is secreted in large quantities from infected cells into the blood where it goes on to cause plasma leakage by disruption of endothelial cells in the vasculature, mediated through direct interaction with endothelial cells and also indirect action by eliciting monocytes to produce cytokines that cause plasma leakage. Indeed, immunization with recombinant NS1 protein or modified NS1 proteins can protect against NS1-mediated vascular leak. However, some antibodies produced against NS1 have cross-reactivity with proteins on endothelial cells, which are hypothesized to further contribute to pathogenesis in the host. For this reason, NS1 is a promising candidate for a DENV vaccine, but for safety reasons care should be taken to avoid eliciting these harmful auto-reactive antibodies. Pursuant to the present invention, the inventors propose to use a highly immunogenic bacteriophage virus-like particle (VLP) platform to display short NS1 peptides as a novel vaccine strategy. This approach holds promise for eliciting high-titer, long-lasting antibodies to NS1 that are specific for epitopes that do not elicit dangerous cross-reactive antibodies. Pursuant to the present invention, the immunogenicity of bacteriophage VLPs displaying NS1 peptides is assessed in mice. In addition, in vitro assessments of the antibodies elicited by the vaccine candidates exhibit their ability to block NS1-mediated endothelial cell disruption and cytokine production. In addition, a pilot in vivo assessment of vaccine candidates pursuant to the present invention using a mouse model of NS1-mediated vascular leakage is performed. Overall, the present invention establishes the feasibility of epitope-specific vaccines against NS1 for eliciting protective and safe antibody responses.

Dengue virus (DENV) is a mosquito-borne virus that can result in potentially fatal hemorrhagic fever. With over half of the world's population at risk of infection, a vaccine that can prevent serious complications is desperately needed. The goal was to use a novel virus-like particle platform to elicit antibodies that can safely block the activity of DENV non-structural protein 1 (NS1), a protein that contributes to the progression to severe complications. Further disclosure and related experiments and results are presented in the attached disclosure.

Dengue virus (DENV) is an arthropod-borne flavivirus with four serotypes (DENV-1-4) that is transmitted by Aedes mosquitos. Every year, approximately 400 million people are infected with dengue virus (DENV), and approximately 10,000 people die as a result of severe DENV disease [1-3]. Additionally, 3 billion people reside in areas that are at risk for contracting DENV, yet a reliable and safe vaccine for population-based vaccinations does not exist. The current DENV vaccine, Dengvaxia by Sanofi-Pasteur has been criticized for its safety efficacy in population-based vaccination campaigns [4]. This is in part due to the unique immune response that follows DENV infection. When an individual is infected with any serotype of DENV, that individual can have a range of illnesses from asymptomatic to fever and severe muscle and bone pain, but they develop antibodies against this serotype. Consequentially, if that individual is infected with a heterologous serotype, the individual is at higher risk for developing severe dengue (SD): dengue hemorrhagic fever (DHF) or dengue shock syndrome (DSS) [5,6]. This is likely due to a phenomenon called Antibody-Dependent Enhancement of infection (ADE), whereby non-neutralizing antibodies to structural proteins present on the DENV virion are able to facilitate infection of Fcγ receptor-expressing cells, leading to increased infection and disease. However, if the individual survives the second infection, they make life-long antibodies that protect from all DENV serotype disease in future exposures. This unique antibody response is why protection against all four DENV serotypes must be addressed in a successful vaccine, while avoiding antibodies that can cause ADE.

DENV has a single-stranded, positive-sense RNA genome that encodes three structural proteins, membrane (M), envelope (E), and capsid I, as well as seven non-structural proteins (NS1, NS2a, NS2b, NS3, NS4a, NS4b, and NS5). Among these non-structural proteins, NS1 has been an attractive target for vaccines. This protein is a 352 amino acid protein that is translated as a monomer during replication of the virus and is glycosylated and forms a dimer in ER of DENV-infected cells [7-11]. This dimeric form is present on both the surface of DENV-infected cells and intracellularly, but NS1 can also be secreted from cells as a hexamer where it can be found circulating in DENV-infected patients. The soluble, hexameric form of NS1 has been associated with inducing vascular leakage of endothelial cells, having a role in autophagy, as well as inducing immune response in peripheral blood mononuclear cells (PBMCs) [12-16]. As such, NS1 has been implicated in playing a role in SD disease. A number of vaccines have been developed against DENV NS1 protein and passive transfer of antibodies against NS1 to naïve mice have been protective in challenge models, suggesting the importance of anti-NS1 antibodies in protection against SD disease [12,17-28]. A thorough review of these vaccine strategies and antibody challenge models can be found in the 2018 review by Chen [29]. Targeting DENV NS1 is attractive because antibodies against NS1 should not be at risk of causing ADE because they are not to a structural protein.

### BRIEF DESCRIPTION OF THE INVENTION

The present invention is directed to a composition comprising a population of virus-like particles derived from a bacteriophage coat protein, often a Qβ or AP205 bacteriophage single coat protein (hereinafter, a Qβ or AP205 virus-like particle or VLP, preferably a Qβ VLP), to which are conjugated onto the surface of the VLP at least one immunogenic peptide comprising an immunogenic peptide determinant (small peptide determinant) of at least 5 and up to 17 contiguous amino acids derived from DENV virus or more often from a conserved region of aligned Dengue virus serotypes 1-4 of NS1 protein. In embodiments, the immunogenic peptide is derived from a non-conserved peptide region of DENV-1-4, often DENV-2. In embodiments, the immunogenic peptide derived from the non-conserved region (often, DENV-2) is often 10-17 amino acid units in length, more often 15 amino acid units in length. In embodiments, the VLPs of the present invention elicit neutralizing antibody responses to all four DENV serotypes. In embodiments, the present invention addresses the failings of prior art vaccines to effectively prevent and/or reduce the likelihood of Dengue virus infections and/or ameliorate the symptoms of dengue fever and/or severe dengue.

In embodiments, the bacteriophage coat protein used to form the VLPs is a Qβ or AP205 bacteriophage single coat protein, preferably a Qβ bacteriophage single coat protein. In embodiments, often 90 single coat protein dimers or 180 bacteriophage single coat proteins self-assemble into a VLP onto the surface of which immunogenic peptides comprising small peptide determinants derived from NS1 protein are conjugated. In embodiments, the immunogenic peptides which are conjugated to the surface of the VLP are derived from Dengue virus (DENV) serotypes 1-4 (Accession #s DENV-1, NP_722461.1; DENV-2, NP_739584.2; DENV-3, YP_001531169.2; and DENV-4, NP_740318.1). In embodiments, these immunogenic peptides are obtained by alignment to show peptides which evidence highly conserved regions for the four serotypes. Alternatively, these peptides are identified through a non-biased approach, when deriving immunogenic peptides from non-conserved regions. In embodiments, the immunogenic peptide which is obtained from non-conserved regions of DENV-2 NS1 polypeptide (DENV-2. NP_739584.2) is one of the 35 polypeptide which is presented in FIG. 10 heref. In embodiments, the immunogenic peptide is one of the nine immunogenic peptides which are presented in FIG. 12B hereof (SEQ ID Nos: 14-22).

Immunogenic peptides which are conjugated to the surface of the VLP according to the present invention are derived from Dengue Virus NS1 protein and are between 5 and 17 contiguous amino acids, 5 and 16 contiguous amino acids, 5 and 15 contiguous amino acids, 5 and 14 contiguous amino acids, 5 and 13 contiguous amino acids, 5 and 12 contiguous amino acids, 6 contiguous amino acids, 7 contiguous amino acids, 8 contiguous amino acids, 9 contiguous amino acids, 10 contiguous amino acids and 11 contiguous amino acids in length. Most often, these immunogenic peptides are derived from NS1 peptide sequences SEQ ID Nos:1-9, often SEQ ID Nos: 1-8, or SEQ ID Nos: 14-22 described herein below. In embodiments, the immunogenic peptide is a peptide presented in FIG. 10A hereof. Often in embodiments, the immunogenic peptides are set forth in FIG. 1 or FIG. 12B hereof.

In embodiments, the immunogenic NS1 small peptide determinant which is conjugated to the VLPs pursuant to the present invention are derived from the following conserved peptide sequences of DENV NS1 peptide:
VHTWTEQYFKQ (SEQ ID NO:1), corresponding to amino acids 25-35 of NS1 protein;
GIRSTTRLENVMWKQI (SEQ ID NO:2), corresponding to amino acids 56-71 of NS1 protein;
EVEDYGFGVFTTNIWLK (SEQ ID NO:3), corresponding to amino acids
   154-170 of NS1 protein;
RLMSAAIKD (SEQ ID NO:4), corresponding to amino acids 182-190 of NS1 protein;
AVHADMGYWIES (Seq ID NO:5), corresponding to amino acids 193-204 of NS1 protein;
WPKSHTLWSNGVLES (SEQ ID NO:6), correspond to amino acids 225-239 of NS1 protein;
RGPSLRTTTVSGK (SEQ ID NO:7), corresponding to amino acids 294-306 of NS1 protein;
GEDGCWYGMEIRP (SEQ ID NO:8), corresponding to amino acids 325-337 of NS1 protein; and
GPWHLGKLELDF (SEQ ID NO:9), corresponding to amino acids 266-277 of NS1 protein; and
KYSWSEWGAK (SEQ ID NO: 10) corresponding to a modification of amino acids 112-122 of NS1 protein with SE substituting for KS in the peptide;
DSGCVVSWKNKELKC (SEQ ID NO: 14) corresponding to amino acids 1-15 ofNS1 protein of DENV-2;
ITDNVHTWTEQYKFQ (SEQ ID NO:15) corresponding to amino acids 21-35 of NS1 protein of DENV-2;
TRLENLMWKQITPEL (SEQ ID NO:16) corresponding to amino acids 61-75 of NS1 protein of DENV-2;
ITPELNHILSENEVK (SEQ ID NO:17) corresponding to amino acids 71-85 of NS1 protein of DENV-2;
AECPNTNRAWNSLEV (SEQ ID NO:18) corresponding to amino acids 141-155 ofNS1 protein of DENV-2;
MIIPKNLAGPVSQHN (SEQ ID NO:19) corresponding to amino acids 241-255 of NS1 protein of DENV-2;
VSQHNYRPGYHTQIT (SEQ ID NO:20) corresponding to amino acids 251-265 of NS1 protein of DENV-2;
TTASGKLITEWCCRS (SEQ ID NO:21) corresponding to amino acids 301-315 ofNS1 protein of DENV-2; and
LRYRGEDGCWYGMEI (SEQ ID NO:22) corresponding to amino acids 321-335 of NS1 protein of DENV-2.

In alternative embodiments, the immunogenic peptide is a peptide which is presented in FIG. 10A hereof. These immune peptides are identified as follows:

PEP 1
SEQ ID NO: 14
DSGCVVSWKNKELKC;
PEP 2
SEQ ID NO: 23
KELKCGSGIFITDNV;
PEP 3
SEQ ID NO: 15
ITDNVHTWTEQYKFQ;
PEP 4
SEQ ID NO: 24
QYKFQPESPSKLASA;
PEP 5
SEQ ID NO: 25
KLASAIQKAHEEGIC;
PEP 6;
SEQ ID NO: 26
EEGICGIRSVTRLEN
PEP 7
SEQ ID NO: 16
TRLENLMWKQITPEL;
PEP 8
SEQ ID NO: 17
ITPELNHILSENEVK;
PEP 9
SEQ ID NO: 27
ENEVKLTIMTGDIKG;
PEP10
SEQ ID NO: 28
GDIKGIMQAGKRSLR;
PEP11
SEQ ID NO: 29
KRSLRPQPTELKYSW;
PEP12
SEQ ID NO: 30
LKYSWKTWGKAKMLS;
PEP13
SEQ ID NO: 31
AKMLSTESHNQTFLI;
PEP14
SEQ ID NO: 32
QTFLIDGPETAECPN;
PEP15
SEQ ID NO: 18
AECPNTNRAWNSLEV;
PEP16
SEQ ID NO: 33
NSLEVEDYGFGVFTT;
PEP17
SEQ ID NO: 34
GVFTTNIWLKLKEKQ;
PEP18
SEQ ID NO: 35
LKEKQDVFCDSKLMS;
PEP19
SEQ ID NO: 36
SKLMSAAIKDNRAVH;
PEP20
SEQ ID NO: 37
NRAVHADMGYWIESA;
PEP21
SEQ ID NO: 38
WIESALNDTWKIEKA;
PEP22
SEQ ID NO: 39
KIEKASFIEVKNCHW;
PEP23
SEQ ID NO: 40
KNCHWPKSHTLWSNG;
PEP24
SEQ ID NO: 41
LWSNGVLESEMIIPK;
PEP25
SEQ ID NO: 19
MIIPKNLAGPVSQHN;
PEP26
SEQ ID NO: 20
VSQHNYRPGYHTQIT;
PEP27
SEQ ID NO: 42
HTQITGPWHLGKLEM;
PEP28
SEQ ID NO: 43
GKLEMDFDFCDGTTV;
PEP29
SEQ ID NO: 44
DGTTVVVTEDCGNRG;
PEP30
SEQ ID NO: 45
CGNRGPSLRTTTASG;
PEP31
SEQ ID NO: 21
TTASGKLITEWCCRS;
PEP32
SEQ ID NO: 46
WCCRSCTLPPLRYRG;
PEP33
SEQ ID NO: 22
LRYRGEDGCWYGMEI;
PEP34
SEQ ID NO: 47
YGMEIRPLKEKEENL;
PEP35
SEQ ID NO: 48
LKEKEENLVNSLVTA.

See also FIG. 1, FIG. 12B, FIG. 10A. In embodiments, the NS1 immunogenic peptides (small peptide determinants) are the peptides represented as SEQ ID Nos: 1-10 and 14-22, often 1-8 and 10, often 1-8 and often 14-22 which are presented herein above. In embodiments, the NS1 immunopeptides (small peptide determinants) according to SEQ ID Nos: 1-9, often 1-8 and 10, often 1-8 or often 14-22, described above are conjugated to VLPs as otherwise described herein, often a Qbeta or AP205 bacteriophage VLP. In embodiments, the NS1 immunopeptides according to SEQ ID Nos: In embodiments, the invention is directed to compositions comprising a population of these VLPs, optionally in combination with a pharmaceutically acceptable carrier, additive and/or excipient. In embodiments, vaccines according to the present invention comprise an amount of VLPs to which immunopeptides are conjugated effective to elicit an immunogenic response, often a protective effect against dengue virus infection in the patient or subject administered said vaccines.

In embodiments, the VLPs are conjugated to the NS1 immunogenic peptides through linkers as described herein. These linkers often comprise a crosslinker as described herein and an oligopeptide linker covalently bonded thereto. The crosslinker and oligopeptide linker may be covalently bonded directly to each other, or optionally through a covalent connector, to form the linker as described herein. The crosslinker is often bonded to the surface of the VLP through a sidechain of an amino acid (often, the butyleneamine sidechain of surface lysine amino acid residues), either directly or through a covalent connector. The crosslinker may be bonded directly to the immunogenic peptide or often to the oligopeptide linker, which is bonded to the immunogenic peptide directly or through a covalent connector. The number of immunogenic peptides which are conjugated to each VLP ranges from less than 1 to more than about 180, 10 to 180, 50 to 180, often 90 to 180 or in certain cases more (e.g often between 90-720, or between 1 and 4 conjugates per coat protein in the VLP).

In embodiments, linkers often are used to conjugate NS1 immunogenic peptides to the surface of the VLPs, from nucleophilic or electrophilic sites on side chains of amino acids of the coat polypeptide of the VLP (e.g. butyleneamine sidechains of lysine residues, among others) to electrophilic or nucleophilic sites (often, the carboxy or amine terminus of the immunogenic peptide or an oligopeptide linker) on the carboxy or amine terminus of the immunogenic peptide which links the immunogenic peptide to the crosslinker or directly to the VLP. In embodiments, often the carboxy terminus of the NS1 immunogenic peptides are linked to an oligopeptide which often comprises between 2 and 15 neutral amino acids, often between 3 and 10 neutral amino acids and terminates in a cysteinyl group which is further bonded to the crosslinker which can be conjugated to the VLP. The oligopeptide linker may be bonded to the NS1 immunopeptide directly or through a covalent connector. Often the oligopeptide comprises a cysteinyl group and between 2 and 5, often 3 or 4 neutral (often glycine) amino acid residues.

In embodiments of the present invention, the oligopeptide linker of the immunogenic peptide conjugate is covalently bonded to an electrophilic or nucleophilic group of the crosslinker (often, a carbonyl group, a vinyl group, a sulfhydryl group, an amine or hydroxyl group) directly or optionally through a covalent connector. Often the oligopeptide linker contains a terminal cysteinyl group which can be used to covalently bind the oligopeptide to the crosslinker. The crosslinker is also bonded to the VLP either directly or through a covalent connector to a nucleophilic or electrophilic group on an amino acid sidechain on the surface of the VLP (often lysine). The oligopeptide may be prepared or modified to facilitate the binding of the oligopeptide and immunogenic peptide to the crosslinker directly (through cysteine or another amino acid containing a functional group) or through a covalent connector. The crosslinker is bonded to the nucleophilic or electrophilic amino acid residues, preferably lysine residues, on the surface of the VLP. The crosslinker may be optionally modified to promote covalent binding between the VLP and the crosslinker (which is linked through the oligopeptide linker to the immunogenic peptide). In embodiments, the oligopeptide of the linker is a 3 to 15 mer, often a 4 to 10 mer oligopeptide comprising neutral amino acid residues bonded to nucleophilic or electrophilic sites of the immunogenic peptide (which is often an amine group or carboxylic acid group of the immunogenic peptide). In embodiments, on one end of the oligopeptide, often the carboxyl terminus, the oligopeptide comprises a cysteinyl group or other amino acid which may be used to link the oligopeptide to the crosslinker. The amino end of the oligopeptide linker may optionally be conjugated to the immunogenic peptide through the use of a covalent connector such as a short amide linker (e.g. a C₁-C₄ alkyl amide group which forms a urea or urethane group with the peptide) or other group, among others. Often the oligopeptide linker is conjugated to the immunogenic peptide by forming a covalent bond directly with the amine terminus or carboxyl terminus of the immunogenic peptide, often the carboxy terminus.

In embodiments, the neutral amino acid residues of the oligopeptide are selected from the group consisting of glycine, alanine, valine, leucine, isoleucine, phenylalanine, tryptophan, methionine, proline, serine and mixtures thereof. In embodiments, the neutral amino acids often are selected from the group consisting of glycine, alanine, valine, leucine, isoleucine, serine and mixtures thereof, more often glycine or alanine, most often glycine. In embodiments, the oligopeptide is GGGC (SEQ ID NO: 13).

In embodiments, a covalent connector group is used to bridge the VLP, the crosslinker, the oligopeptide linker and/or the immunogenic peptide depending principally upon the functional groups on the amino acid residues of the VLP and the crosslinker. Often the amino acid residues on the VLP are lysine residues and the crosslinker may vary, but often is a (Succinimidyl 6-((beta-maleimidopropionamido)hexanoate)) (SMPH) crosslinker.

Common connector groups which are used in the present invention include the following chemical groups:

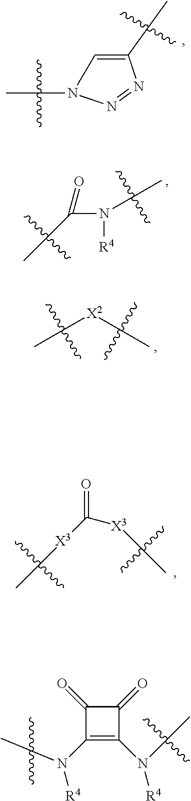

Where X² is O, S, NR⁴, C(O), S(O), S(O)₂, —S(O)₂O, —OS(O)₂, or OS(O)₂O;
X³ is O, S, NR⁴; and
R⁴ is H, a C₁-C₃ alkyl or alkanol group, or a —C(O)(C₁-C₃) group. The amide group, indicated above, is a preferred connector group. In each connector group, the connector group may be extended with a C₁-C₄ alkylene group at a point of attachment to bind to a VLP, crosslinker, oligopeptide and/or immunogenic peptide (e.g, preferably between the amino acid residue on the VLP and the crosslinker, between the crosslinker and the oligopeptide of the linker or between the oligopeptide of the linker and the immunogenic peptide).

In embodiments, in the composition according to the present invention the immunogenic peptide conjugate is displayed at one or more nucleophilic or electrophilic amino acid residues on the surface of the VLP, often at a plurality of lysine residues on the surface of the VLP. In embodiments, the immunogenic peptide conjugate is displayed on the bacteriophage at the lysine residues by covalently binding an immunogenic peptide as described herein to the lysine residues through a crosslinker group and optional connector group. In embodiments, the linker group comprises a 3 to 15 mer, preferably a 4 to 10 mer oligopeptide linker covalently bonded to a crosslinker which is bonded to the VLP particle as described herein. Often the linker group comprises a cysteinyl group at the carboxyl end of the oligopeptide and 2 to 5 neutral amino acid residues, often 3 or 4 glycine amino acid residues.

In embodiments of the present invention, the oligopeptide linker of the immunogenic peptide conjugate is covalently bonded to an electrophilic or nucleophilic group of the crosslinker (e.g. a carbonyl group, a vinyl group, an amine or hydroxyl group) which optionally has been modified to facilitate the binding of the oligopeptide and immunogenic peptide to the crosslinker through a covalent connector and the crosslinker is bonded to the nucleophilic or electrophilic amino acid residues, preferably lysine residues on the surface of the bacteriophage through the crosslinker, which may be optionally modified with a covalent connector to promote covalent binding between the bacteriophage and the crosslinker (which is often linked through the oligopeptide linker to the immunogenic peptide). In embodiments, the oligopeptide of the linker is a 3 to 15 or 4 to 15 mer, preferably a 4 to 10 mer oligopeptide comprising neutral amino acid residues which are bonded to nucleophilic or electrophilic sites of the immunogenic peptide (which is often an amine group or carboxylic acid group of the immunogenic peptide).

In embodiments, on one end of the oligopeptide linker, often the carboxyl terminus, the oligopeptide linker comprises a cysteinyl group or other amino acid which may be used to link the oligopeptide linker to the crosslinker. The amino end of the oligopeptide may optionally be conjugated to the peptide through the use of a short amide linker (e.g. a C₁-C₄ alkyl amide group which forms a urea or urethane group with the peptide or other group, among others. At the other end of the oligopeptide linker, often the oligopeptide linker is conjugated to the immunogenic peptide by forming a covalent bond with the amine terminus or carboxyl terminus of the immunogenic peptide.

In embodiments, the neutral amino acid residues are selected from the group consisting of glycine, alanine, valine, leucine, isoleucine, phenylalanine, tryptophan, methione, proline, serine and mixtures thereof. In embodiments, the neutral amino acids often are selected from the group consisting of glycine, serine and mixtures thereof, more often glycine. In embodiments, the crosslinker is (Succinimidyl 6-((beta-maleimidopropionamido)hexanoate)) (SMPH) and the oligopeptide comprises three glycine amino acid residues and a cysteine amino acid residue at the carboxy end of the NS1 short immunopeptide, wherein the sulfhydryl group of the cysteine residue binds to the crosslinker.

In embodiments, the immunogenic peptide comprises at least five contiguous amino acids of the immunogenic peptide sequence of a NS1 immunogenic peptide, often at least five contiguous amino acids from an immunogenic peptide of SEQ ID Nos 1-9, often 1-8 or 10 or 14-22 hereof or as otherwise disclosed herein. In embodiments, the immunogenic peptide is a peptide according to the sequence of SEQ ID Nos 1-9, 1-8 and 10 or 14-22 as described herein.

In embodiments, the present invention is directed to a population of virus-like particles (VLPs) derived from Qbeta or AP205 bacteriophages to which NS1 short chain immunopeptides are conjugated as otherwise described herein. In embodiments, the population of VLPs is combined with a pharmaceutical carrier, additive and/or excipient to provide immunogenic compositions according to the present invention. In embodiments the population of VLPs is formulated into a pharmaceutical composition as a vaccine formulation (vaccine) for immunizing a patient or subject against a dengue virus infection. In embodiments, the pharmaceutical composition comprises an adjuvant or other active component to enhance or facilitate an immunogenic response in a patient or subject to which the composition is administered.

In embodiments, in the composition according to the present invention which comprises a population of VLPs, the immunogenic NS1 peptide is displayed at one or more nucleophilic or electrophilic amino acid residues on the surface of the VLP, preferably at a plurality of lysine residues (often the butyleneamine side chain of lysine) on the surface of the VLP. In embodiments, the immunogenic peptide is displayed on the bacteriophage at lysine residues on the coat polypeptide by covalently binding an immunogenic amino acid derived from conserved regions of the NS1 protein of DENV to the lysine residues of the coat polypeptide through a linker group which includes a crosslinker and an oligopeptide linker.

In embodiments, the oligopeptide linker group comprises a 4 to 15 mer, preferably a 4 to 10 mer oligopeptide covalently bonded to a crosslinker as described herein.

In embodiments, the present invention is directed to a pharmaceutical composition comprising a population of virus-like particles as described herein in combination with a pharmaceutically acceptable carrier, additive and/or excipient, or alone. In embodiments, the composition is formulated for administration to a subject or patient as a vaccine for enhancing immunogenicity of the patient or subject to Dengue virus. In embodiments the pharmaceutical composition or vaccine comprises an effective amount of an adjuvant (e.g., Advax, MF 59, CPG 1018, AS01B, AS03, AS04, etc.).

In embodiments, the present invention is directed to a method for enhancing an immune response against a dengue virus (DENV) infection in a patient or subject in need comprising introducing a pharmaceutical composition comprising a population of VLPs as otherwise described herein to said subject or patient, wherein an enhanced immune response against said dengue virus (DENV) infection is produced in said patient or subject. In embodiments, the present invention is directed to a method for reducing the likelihood of a dengue virus (DENV) infection and/or morbidity in a patient or subject in need. In embodiments, the present invention is directed to a method wherein the composition is prophylactic for a dengue virus (DENV) infection.

In embodiments, the present invention is directed to a method of inducing an immunogenic response in a patient or subject comprising administering a composition comprising an effective amount of a population of immunogenic peptide VLPs as otherwise described herein to said patient or subject.

In embodiments, the present invention is directed to a method for treating or inhibiting a dengue virus (DENV) infection, morbidity, or a symptom thereof in a patient or subject in need comprising administering to said patient or subject a composition comprising an effective amount of a population of immunogenic peptide conjugated VLPs as otherwise described herein to said patient or subject.

In embodiments, the present invention is directed to a method of identifying immunogenic peptides and/or epitopes which can be used to raise an immunogenic response to a dengue virus (DENV) infection in a patient or subject. These methods are disclosed in the detailed description of the invention and the examples, set forth herein.

In embodiments, the infection is a dengue virus (DENV) infection and the symptom is a symptom associated with said infection, such as one or more of high fever, aches and pains across the body, nausea, vomiting, skin rash, loss of appetite, headache, abdominal pain, bloody gums and nose, blood in stools, blood vessel damage, organ dysfunction in heart, lungs and/or liver, blood in vomit, bruise-like formations on the skin and bleeding under the epidermis, among others.

In embodiments, the present invention is directed to a method for treating or reducing the likelihood of a dengue virus (DENV) infection, morbidity, or a symptom thereof in a patient or subject in need comprising administering to said patient a composition comprising an effective amount of a population of immunogenic peptide conjugated VLPs as otherwise described herein to said patient or subject. In embodiments, the symptom is one or more of high fever, aches and pains across the body, nausea, vomiting, skin rash, loss of appetite, headache, abdominal pain, bloody gums and nose, blood in stools, blood vessel damage, organ dysfunction in heart, lungs and/or liver, blood in vomit, bruise-like formations on the skin and bleeding under the epidermis, among others. In embodiments, a patient or subject is immunized against a dengue virus infection by administering a vaccine according to the present invention which optionally includes an adjuvant as disclosed herein.

In embodiments, the present invention is therefore directed to vaccines which target dengue virus (DENV) infection or a symptom or morbidity thereof in a patient or subject in need as otherwise disclosed herein for prophylactic and/or therapeutic purposes.

### BRIEF DESCRIPTION OF THE FIGURES

FIG. 1 shows the conservation of NS1 proteins from four DENV serotypes. The amino acid sequence of the four DENV serotypes (Accession #s: DENV-1 (NP_722461.1), DENV-2 (NP 739584.2), DENV-3 (YP_001531169.2), and DENV-4 (NP_740318.1)) were aligned and perfectly conserved amino acids are shown in red. Blue squares indicate the highly conserved regions chosen for investigation as epitope-based vaccine candidates against DENV-NS1, with numbers designating amino acid numbers.

FIG. 2 shows that bacteriophage VLPs elicit high titer and long lasting antibodies in a single dose. (A) A peptide of interest was conjugated to Qβ VLPs using SMPH bifunctional crosslinker or KLH. Groups of mice (n=6) were immunized intramuscular with Qβ-PEP (5 ug), KLH-PEP (15 ug) or KLH-PEP (25 ug). Serum was collected at 1-, 2-, and 3-weeks post immunization and assessed for PEP-specific IgG by ELISA. (B) Mice (n=5) were immunized intramuscularly with 1 or 3 doses with bacteriophage VLPs displaying a peptide representing a Human Papillomavirus neutralizing epitope, and peptide-specific IgG titers were measured by ELISA for nearly 2-years post-vaccination.

FIG. 3 shows the conjugation of peptides to Qβ VLPs. In a preferred embodiment, ninety coat protein dimers (B) self-assemble into Qβ VLPs (A) when expressed in E. coli. Qβ VLPs are then purified and mixed with SMPH bi-functional crosslinker which reacts with lysines on the surface of the VLPs (yellow space-fill in A and B). Then, synthetic peptide is mixed with the SMPH-VLP which conjugates the peptide to the surface of the VLP by a reaction of SMPH to the sulfhydryl group of a terminal cysteine residue. Successful conjugation is confirmed by observation of a series of increased molecular weight bands in a Coomassie-stained SDS-PAGE gel, representing various numbers of peptides successfully conjugated to the Qβ coat protein (C, example of a successful conjugation).

FIG. 4 shows that vaccine-elicited antibodies that bind to HUVEC cells exhibit fluorescence by confocal microscopy (B), while vaccine-elicited antibodies that do not cross-react with HUVEC cells do not exhibit fluorescence (A).

FIG. 5 shows (A) that NS1 directly disrupts tight junctions on endothelial cells through an as-yet-undefined mechanism. (B) shows that NS1 signals through TLRs on PBMCs, resulting in IL-6, IL-10, and TNF-α production which can go on to cause vascular leakage. Antibodies elicited by the vaccine candidates are tested to see if they block these two activities of NS1.

FIG. 6 shows conserved peptides mapped to the structure of DENV-2 NS1 protein dimer. The DENV-2 NS1 dimer PDB file was imported into Cn3D structure visualization program with each peptide colored as indicated. Different views are shown with space-fill and ribbon structures. Pink dashed lines indicate the assumed location of conserved peptide 112-122, which corresponds to an unresolved disordered region of the NS1 protein.

FIG. 7 shows Qβ VLPs displaying NS1 peptides are immunogenic in mice. Mice (n=5/group) were immunized with 5 μg/mouse with Qβ-NS1-PEP immunogens or Qβ (VLPs alone, control, black lines) two times at 3-week intervals. Sera were collected and tested three weeks after the first immunization (day 21, red lines) and three weeks after the second immunization (day 42, blue lines). Sera were tested at various dilutions for binding to their cognate peptide using a peptide ELISAs.

FIG. 8 shows Qβ-VLP-NS1-PEP vaccine-elicited sera binding to soluble hexameric NS1. (A) DENV-1 NS1, (B) DENV-2 NS1, (C) DENV-3 NS1, (D) DENV-4 NS1.

FIG. 9 shows the binding of Qβ-VLP-NS1-PEP elicited antibodies to DENV-infected HEK293 cells. Cells were infected at a MOI of 100 for 72 h with DENV-2 NGC. Cells were then fixed with methanol and stained to detect DENV-infected cells (DENV Env, Red in Merge), sera from a Qβ-112-122-immunized mouse (Mouse anti-sera, Green in Merge) and nuclear staining with Hoechst (blue). A representative image generated with 1:320 dilution of sera from Qβ-112-122 or 1:40 dilution of sera from Qβ alone are shown here, along with a commercially available NS1 antibody as a positive control.

FIG. 10 shows an unbiased design of DENV Qβ VLP vaccines. The sequence of DENV-2 was separated into peptides 15 amino acids in length, and overlapped by 5 amino acids on each end (A). A total of 35 peptides were manufactured and top peptides were highlighted in yellow (A). Peptides were manufactured with a linker sequence containing a terminal cysteine and conjugated to the exposed lysines (yellow) on the surface of Qβ VLPs using the bifunctional crosslinker SMPH (B).

FIG. 11 shows the screening of the DENV peptide library. 6-8 week old female BALB/c mice (n=2/group) were immunized twice with 5 ug/50 uL of Qβ VLP vaccines on day 0 and day 21 (A). 42 days post immunization, mice were sacrificed and sera was collected for antibodies (A). ELISAs against cognate peptide were performed; dotted line represents 1:160 dilution of sera and indicates the cut off for acceptable antibody titers (B). PEP library was screened for binding to soluble, hexameric DENV-2 NS1. Dotted line represents the cut off for acceptable titers at 1:1600 (C).

FIG. 12 shows the immunization of BALB/c mice with top 9 Qβ VLP vaccines (conjugating immunogenic peptides of SEQ ID NO: 14-22) induce high titer antibodies against cognate peptide. The top nine Qβ-PEP vaccines were identified from the initial screening and highlighted on the dimeric structure of DENV-2 NS1 (A). The top nine Qβ-PEP vaccine amino acid positions and peptide sequences are displayed in the table (B). 6-8 week old BALB/c mice were immunized with Qβ-PEP vaccines from the top nine binders n=6 mice/vaccine (3 male, 3 female). Peptide ELISA was used to test binding of against cognate peptide comparing vaccine-sera (blue) and control sera of mice immunized with Qβ only (red) (C).

FIG. 13 shows that the top 9 VLP vaccines (which conjugate immunogenic peptides SEQ ID Nos. 14-22) against DENV NS1 bind DENV-infected cells and hexameric forms of DENV NS1. Human embryonic kidney (HEK) 293 cells were infected with DENV-2 NGC Proto at an MOI of 100. Infected cells were then fixed and stained with anti-DENV NS1 sera from vaccinated mice (green) or positive control monoclonal anti-flavivirus NS1 antibody (green), and rabbit anti-flavivirus envelope protein antibody (4G2) for detection of viral infection (red) (A). Binding of top 9 VLP vaccines to soluble hexameric NS1 from DENV-2 (B). Pooled sera samples of top 9 vaccines against soluble, hexameric NS1 from DENV-1, DENV-3, and DENV-4 (C).

FIG. S1 shows the predicted exposure of conserved regions of DENV NS1 protein. The space-filled (left) and ribbon structure (right) of each conserved region is highlighted in red. Not shown is the structure of Qβ-VLP-PEP 112-122 due to it being an unresolved disordered region of the NS1 protein.

FIG. S2 shows the conjugation of peptides to Qβ-VLPs. To ensure the successful conjugation of peptides to Qβ-VLPs, vaccine candidates were run on SDS-PAGE gels. Laddering effect shows heavier molecular weight when peptides are conjugated to the Qβ-VLPs.

FIG. S3 shows the binding of pooled Qβ-VLP-PEP antibodies to soluble, hexameric NS1. Sera collected from Qβ-VLP-PEP-immunized mice on day 42 (n=5) were pooled and used in NS1 ELISAs to determine the binding efficacy of these antibodies to the hexameric form of DENV NS1 from all four serotypes.

FIG. S4 shows the fluorescent microscopy of Qβ-VLP-PEP antibodies binding to DENV-infected cells. HEK-293 cells were infected with DENV-2 at an MOI of 100 for 3 days. Cells were fixed and stained with a commercial pan-flavivirus 4G2 antibody targeting envelope protein, as well as Qβ-VLP-PEP antibodies from immunized mice. Images are representative samples of individual mouse sera from each treatment group.

### DETAILED DESCRIPTION OF THE INVENTION

In accordance with the present invention there may be employed conventional molecular biology, microbiology, and recombinant DNA techniques within the skill of the art. Such techniques are explained fully in the literature. See, e.g., Sambrook et al, 2001, “Molecular Cloning: A Laboratory Manual”; Ausubel, ed., 1994, “Current Protocols in Molecular Biology” Volumes I-III; Celis, ed., 1994, “Cell Biology: A Laboratory Handbook” Volumes I-III; Coligan, ed., 1994, “Current Protocols in Immunology” Volumes I-III; Gait ed., 1984, “Oligonucleotide Synthesis”; Hames & Higgins eds., 1985, “Nucleic Acid Hybridization”; Hames & Higgins, eds., 1984, “Transcription And Translation”; Freshney, ed., 1986, “Animal Cell Culture”; IRL Press, 1986, “Immobilized Cells And Enzymes”; Perbal, 1984, “A Practical Guide To Molecular Cloning.”

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a,” “and” and “the” include plural references unless the context clearly dictates otherwise.

Furthermore, the following terms shall have the definitions set out below.

The term “patient” or “subject” is used throughout the specification within context to describe an animal, generally a mammal and preferably a human, to whom treatment, including prophylactic treatment (prophylaxis), with the immunogenic compositions and/or vaccines according to the present invention is provided. For treatment of those infections, conditions or disease states which are specific for a specific animal such as a human patient, the term patient refers to that specific animal. In most instances, the patient or subject of the present invention is a human patient of either or both genders.

The term “effective” is used herein, unless otherwise indicated, to describe a number of VLP's or an amount of a VLP-containing composition which, in context, is used to produce or effect an intended result, whether that result relates to the prophylaxis and/or therapy of a flavivirus infection, especially a Dengue virus infection as otherwise described herein. The term effective subsumes all other effective amount or effective concentration terms (including the term “therapeutically effective”) which are otherwise described or used in the present application.

The term “Flavivirus” is used to describe a small, positive sense RNA virus that can cause yellow fever, dengue, Japanese encephalitis, and West Nile virus in addition to tick-borne encephalitis. These pathogens principally use mosquitoes from the Aedes genus as vectors. They are found in both temperate and tropical areas. The specificity of their natural hosts determines that flaviviruses are found in areas specific to their hosts rather than being equally distributed around the globe. Other Flaviviruses are transmitted by ticks and are responsible for encephalitis and hemorrhagic diseases: Tick-borne Encephalitis (TBE), Kyasanur Forest Disease (KFD) and Alkhurma disease, and Omsk hemorrhagic fever. Globally, of the flaviviruses, the dengue virus (DENV) has the most impact with about 50-100 million infections per year which result in more than 20,000 deaths annually. Although there are effective vaccines for yellow fever, Japanese encephalitis, and tick born encephalitis, an effective dengue vaccine has yet to be discovered until the present invention. The delay in the dengue vaccine comes from concerns that the vaccines appear to predispose the subject to infections from other strains of dengue virus, a problem which is addressed by the present invention.

The term “dengue virus (DENV) infection” is used to describe an infection which occurs as a consequence of infection by the dengue virus (DENV) pathogen. In its less debilitating or typical form a dengue virus (DENV) infection is often labeled “dengue fever” and in its more severe form is labeled “severe dengue”. Some patients with dengue fever show no symptoms at all. Neonates and young children infected with the dengue virus typically have mild symptoms such as a fever and a rash over their entire bodies, but no other symptoms of dengue. Older children and adults may also have these mild symptoms, or they may have classic symptoms of dengue, including a high fever that lasts for two days to a week or more, severe pain in the muscles, bones, and joints, pain behind the eyes, severe headaches, nausea and vomiting and a rash. Dengue fever is characterized by a fever response with two peaks. Near the beginning of the infection, the patient experiences a very high body temperature, which then starts to drop and suddenly climbs again for a second time.

Other symptoms of dengue fever include a decrease in the number of white blood cells and a low level of platelets in the blood. Patients with dengue fever may have skin hemorrhages (bleeding under the surface of the skin) that appear as red or purple spots on the body. Dengue fever can also cause bleeding from the skin, nose, and gums. Recovery from dengue fever is often lengthy, lasting several weeks, and patients can experience lingering fatigue and depression.

In the case of severe dengue, infection occurs which is more serious than dengue fever. Although the early symptoms of severe dengue are similar to dengue fever, severe dengue has a much higher death rate. As with dengue fever, patients with severe dengue have a high fever, experience bleeding, and have a reduced white blood cell count. The major symptom of severe dengue is leakage of blood plasma out of the capillaries. This leakage occurs 24 to 48 hours after the patient's fever drops, a period doctors refer to as the critical phase. Patients who improve after their fever drops are said to have dengue, but patients who deteriorate have severe dengue. In people with severe dengue, the escape of the plasma from the circulatory system can cause fluids to collect in body cavities. Plasma leakage can be detected by the caregiver observing a higher-than-normal concentration of red blood cells and an abnormally low protein level in the blood. Severe bleeding can also occur. In certain instances, stomach and intestinal bleeding can cause death. In addition, patients with severe dengue have a tendency to bruise easily and experience changes in blood pressure and pulse rate. Most patients recover from severe dengue with intravenous fluid replacement. The loss of plasma and protein however, can cause the patient to experience a condition called shock. Patients in shock show signs of circulatory failure. The lack of blood circulation causes the patient to have cold, clammy, bluish skin. Patients experiencing shock seem restless, and their blood pressure and pulse may be undetectable. Severe dengue can also lead to respiratory distress and injury of other organs. If untreated, shock can lead to death within 24 hours, but if treated quickly with intravenous fluid replacement, patients can recover.

Use of the present compositions can provide protection and/or therapy from dengue virus infections (e.g. dengue fever or severe dengue fever) and protection and/or reduction in the likelihood of a dengue virus (DENV) infection or one or more of the symptoms associated with dengue fever or severe dengue fever as described hereinabove.

As used herein, “epitope” refers to an antigenic determinant of a polypeptide. An epitope could comprise 3 amino acids in a spatial conformation which is unique to the epitope. Generally an epitope consists of at least 4 such amino acids, and more often, consists of at least 5-10 or more such amino acids. Methods of determining the spatial conformation of amino acids are known in the art, and include, for example, x-ray crystallography and 2-dimensional nuclear magnetic resonance.

As used herein, the term “virus-like particle of a bacteriophage” refers to a virus-like particle (VLP) resembling the structure of a bacteriophage, being non-replicative and noninfectious, and lacking at least the gene or genes encoding for the replication machinery of the bacteriophage, and typically also lacking the gene or genes encoding the protein or proteins responsible for viral attachment to or entry into the host.

This definition should, however, also encompass virus-like particles of bacteriophages, in which the aforementioned gene or genes are still present but inactive, and, therefore, also leading to non-replicative and noninfectious virus-like particles of a bacteriophage.

VLP of RNA bacteriophage coat protein: The capsid structure formed from the self-assembly of one or more subunits of RNA bacteriophage coat protein and optionally containing host RNA is referred to as a “VLP of RNA bacteriophage coat protein”. In a particular embodiment, the capsid structure is formed from the self assembly of 90 coat protein single-chain dimers or 180 coat protein monomers. In embodiments, 90 coat protein dimers or 180 coat protein monomers typically self-assemble into a VLP. In preferred embodiments, 90 Qβ coat protein dimers self-assemble into a Qβ VLP to which are conjugated immunogenic polypeptides as otherwise described herein. In alternative embodiments, 180 Qβ monomeric coat proteins self-assemble into a Qβ VLP to which are conjugated immunogenic polypeptides as otherwise described herein.

As used herein, the term “coat protein(s)” refers to the protein(s) of a bacteriophage or a RNA-phage capable of being incorporated within the capsid assembly of the bacteriophage or the RNA-phage. These include, but are not limited to Qβ, AP205, PP7, MS2, AP205, R17, SP, PP7, GA, M11, MX1, f4, Cb5, Cb12r, Cb23r, 7s and f2 RNA bacteriophages. Coat proteins which are used in the present invention include coat proteins from bacteriophages often include Qβ or AP205. Most often Qβ coat polypeptides are used to create conjugated VLPs according to the present invention. In embodiments, Qβ coat polypeptides are often dimeric wherein 90 dimeric coat polypeptides self-assemble into VLPs to which immunogenic peptides are conjugated. In alternative embodiments, Qβ coat polypeptides are monomeric wherein 180 monomeric Qβ coat polypeptides self-assemble into VLPs to which immunogenic peptides are conjugated.

As used herein, a “coat polypeptide” as defined herein is a polypeptide of the full length coat protein of the bacteriophage, a polypeptide fragment of the coat protein that possesses coat protein function and additionally encompasses the full length coat protein as well or single-chain variants thereof.

As used herein, the term “immune response” refers to a humoral immune response and/or cellular immune response leading to the activation or proliferation of B- and/or T-lymphocytes and/or antigen presenting cells. In some instances, however, the immune responses may be of low intensity and become detectable only when using at least one substance in accordance with the invention. “Immunogenic” refers to an agent used to stimulate the immune system of a living organism, so that one or more functions of the immune system are increased and directed towards the immunogenic agent. An “immunogenic peptide”, “immunogenic NS1 peptide” or “small peptide determinant” is a conjugated NS1 peptide that elicits a cellular and/or humoral immune response as described above, whether alone or linked to a carrier in the presence or absence of an adjuvant. Preferably, antigen presenting cells may be activated. In embodiments, VLP compositions according to the present invention show immunogenicity to Dengue virus (DENV), but exhibit a low or even an absence of immugenicity to endothelial cells of the patient or subject to whom compositions according to the present invention have been administered. In embodiments, VLP compositions according to the present invention, in contrast to prior art immunogenic and vaccine compositions, are tetravalent and elicit broadly neutralizing long-lasting antibody responses to all four DENV serotypes (1-4), and inhibit and/or eliminate secondary infection such as Severe Dengue, including Dengue Hemorrhagic Fever and Dengue Shock Syndrome which occurs as a consequence of “Antibody Dependent Enhancement of Infection.”

The term “immunogenic peptide conjugate” or peptide conjugate refers to an immunogenic peptide of between 5 and 17 amino acid residues in length which comprises a small peptide antigenic determinant (epitope) derived from DENV NS1 protein as otherwise described herein and is conjugated to the external surface of a VLP, often a Qβ or AP205 bacteriophage VLP, often a Qβ bacteriophage VLP through a linker molecule to a nucleophilic or electrophilic (often a nucleophilic) amino acid on the surface of the bacteriophage. In embodiments, the nucleophilic amino acid is a lysine residue on the surface of the VLP. The immunogenic peptide is conjugated to the bacteriophage VLP through a linker molecule. Often the linker molecule comprises a 3-15 mer, often a 4-12 mer, a 4-10 mer, a 4-8 mer a 4-6 mer or a 4 mer oligopeptide (preferably comprising neutral amide acid residues and a cysteinyl residue) which is covalently bonded to a crosslinker molecule at one end and the immunogenic peptide on the other end as described herein to form the linker. Accordingly, the oligonucleotide is covalently linked at one end to the immunogenic peptide often through an electrophilic or nucleophilic functional group on the immunogenic peptide (often a carboxyl group or amine group, more often an amine group which is optionally further linked by an amide or other group, often a short, C₁-C₄ alkyl amide) and on the other end to the crosslinker, which further links the VLP to the oligopeptide and the immunogenic peptide.

As used herein, the term “vaccine” refers to a formulation which contains the composition of the present invention and which is in a form that is capable of being administered to an animal, often a human patient or subject.

The term “valency” is used to describe the density of the immunogenic peptide conjugates displayed on VLPs according to the present invention. Valency in the present invention may range from low valency (“low density”) to high valency (“high density”), from less than 1 to more than about 180, preferably 90 to 180 or in certain cases more (e.g between 90-720, or from 1 to 4 conjugates per coat protein in the VLP). Immunogenic compositions according to the present invention comprise VLPs which are preferably high valency and comprise VLPs which display at least 50-60 up to about 180 or more, often 50-180 or more, more often 90-180 or more crosslinked conjugated immunogenic peptides per VLP as otherwise described herein. In embodiments, at least 90 immunogenic peptide conjugates on a VLP are considered “high density” because the display of 90 copies of antigen/immunogenic peptide on the surface of the VLP produces high titer antibodies.

A nucleic acid molecule is “operatively linked” to, or “operably associated with”, an expression control sequence when the expression control sequence controls and regulates the transcription and translation of nucleic acid sequence. The term “operatively linked” includes having an appropriate start signal (e.g., ATG) in front of the nucleic acid sequence to be expressed and maintaining the correct reading frame to permit expression of the nucleic acid sequence under the control of the expression control sequence and production of the desired product encoded by the nucleic acid sequence. If a gene that one desires to insert into a recombinant DNA molecule does not contain an appropriate start signal, such a start signal can be inserted in front of the gene.

It should be appreciated that also within the scope of the present invention are nucleic acid sequences encoding the polypeptide(s) and/or oligopeptides of the present invention, which code for a polypeptide or oligopeptide having the same amino acid sequence as the sequences disclosed herein, but which are degenerate to the nucleic acids disclosed herein. By “degenerate to” is meant that a different three-letter codon is used to specify a particular amino acid.

The term “crosslinker” or “crosslinking agent” refers to a chemical compound used to covalently bind, or conjugate, biomolecules together, such as an oligopeptide to a VLP or an oligopeptide to an immunogenic peptide or alternatively, directly to the VLP or the immunogenic peptide. The term “protein crosslinking” refers to utilizing protein crosslinkers to conjugate peptides or proteins together. Crosslinking agents for use herein possess reactive moieties specific to various electrophilic or nucleophilic functional groups (e.g., sulfhydryls, amines, carbohydrates, carboxyl groups, hydroxyl groups, carbonyls, etc.) on proteins, peptides, or other molecular complexes or molecules such as opioids as described herein. The atoms separating a crosslinker agent's reactive groups, and eventually the conjugated oligopeptide/VLP or oligopeptide/immunogenic peptide form the “spacer arm”.

A zero-length crosslinker refers to protein crosslinkers that join two molecules without adding additional spacer arm atoms. Homobifunctional crosslinker reagents have the same reactive group on both ends of the spacer arm (i.e., Amine Reactive-Amine Reactive); while heterobifunctional crosslinkers have different reactive groups on each end of a spacer arm (i.e., Sulfhydryl Reactive-Amine Reactive). It is noted that in addition to the following crosslinking agents, additional short-chain crosslinking agents such as short-chain alkyl amides (CH₂)ᵢC(O)NH₂, (CH₂)ᵢC(O), C(O)(CH)ᵢC(O), NHC(O)(CH₂)ᵢC(O) or NHC(O)(CH₂)ᵢC(O)NH groups where i is from 1 to 4 or connector groups as otherwise described herein, can be used to link an immunogenicpeptide to an oligopeptide or a crosslinker to a lysine group on the VLP. The following crosslinking agents are exemplary for use in the present invention:
ANB-NOS (N-5-Azido-2-nitrobenzoyloxysuccinimide)
BMPS N-(β-Maleimidopropyloxy)succinimide ester
EMCS (N-[e-Maleimidocaproyloxy]succinimide ester)
GMBS (N-[Gamma-Maleimidobutyryloxy] Succinimide)
LC-SPDP Succinimidyl 6-(3-[2-pyridyldithio]-propionamido)hexanoate)
MBS (m-Maleimidobenzoyl-N-hydroxysuccinimide ester)
PDPH (3-[2-Pyridyldithio]propionyl hydrazide)
SBA (N-Succinimidyl bromoacetate)
SIA (N-Succinimidyl iodoacetate)
Sulfo-SIA N-Sulfosuccinimidyl iodoacetate)
SMCC (Succinimidyl-4-[N-maleimidomethyl]cyclohexane-1-carboxylate)
SMPB (N-Succinimidyl 4-[4-maleimidophenyl]butyrate)
SMPH (Succinimidyl-6-[β-maleimidopropionamido]hexanoate)
SPDP (N-Succinimidyl 3-[2-pyridyldithio]-propionate)
Sulfo-LC-SPDP Sulfosuccinimidyl 6-(3′-[2-pyridyldithio]-propionamido)hexanoate
Sulfo-MBS (m-Maleimidobenzoyl-N-hydroxysulfosuccinimide ester)
Sulfo-SANPAH (N-Sulfosuccinimidyl-6-[4′-azido-2′-nitrophenylamino] hexanoate)
sulfo-SMCC (Sulfosuccinimidyl-4-[N-maleimidomethyl]cyclohexane-1-carboxylate)
BS2G (Bis[Sulfosuccinimidyl] glutarate)
BS3 (Bis[sulfosuccinimidyl] suberate)
DSG (Disuccinimidyl glutarate)
DSP (Dithiobis[succinimidyl propionate])
DSS (Disuccinimidyl suberate)
DSSeb (Disuccinimidyl sebacate)
DST (Disuccinimidyl tartrate)
DTSSP (3,3′-Dithiobis[sulfosuccininidylpropionate])
EGS (Ethylene glycolbis(succinimidylsuccinate)
Sulfo-EGS Ethylene glycolbis(sulfosuccinimidylsuccinate)
CDI (N,N′-Carbonyldiimidazole)
DCC (N,N′-dicyclohexylcarbodiimide)
EDC-HCl 1-Ethyl-3-[3-dimethylaminopropyl]carbodiimide hydrochloride)
NHS (N-hydroxysuccinimide) and
Sulfo-NHS (N-hydroxysulfosuccinimide).

Preferred crosslinkers for use in the present invention are heterobifunctional agents which are capable of linking Amine-to-Sulfhydryl groups. Exemplary crosslinking agents include:
SIA (succinimidyl iodoacetate)
SBAP (succinimidyl 3-(bromoacetamido)propionate)
SIAB (succinimidyl (4-iodoacetyl)aminobenzoate)
Sulfo-SIAB (sulfosuccinimidyl (4-iodoacetyl)aminobenzoate)
AMAS (N-α-maleimidoacet-oxysuccinimide ester)
BMPS (N-β-maleimidopropyl-oxysuccinimide ester)
GMBS (N-γ-maleimidobutyryl-oxysuccinimide ester)
MBS (m-maleimidobenzoyl-N-hydroxysuccinimide ester)
SMCC (succinimidyl 4-(N-maleimidomethyl)cyclohexane-1-carboxylate)
EMCS (N-ε-malemidocaproyl-oxysuccinimide ester)
Sulfo-GMBS (N-γ-maleimidobutyryl-oxysulfosuccinimide ester)
Sulfo-MBS (m-maleimidobenzoyl-N-hydroxysulfosuccinimide ester)
Sulfo-SMCC (sulfosuccinimidyl 4-(N-maleimidomethyl)cyclohexane-1-carboxylate)
Sulfo-EMCS (N-ε-maleimidocaproyl-oxysulfosuccinimide ester)
Sulfo-SMPB (sulfosuccinimidyl 4-(N-maleimidophenyl)butyrate)
SMPB (succinimidyl 4-(p-maleimidophenyl)butyrate)
SMPH (Succinimidyl 6-((beta-maleimidopropionamido)hexanoate))
LC-SMCC (succinimidyl 4-(N-maleimidomethyl)cyclohexane-1-carboxy-(6-amidocaproate)) and
Sulfo-KMUS (N-κ-maleimidoundecanoyl-oxysulfosuccinimide ester).
In embodiments, (Succinimidyl 6-((beta-maleimidopropionamido)hexanoate)) (SMPH) is a crosslinker most often used.

As used herein, the term “polynucleotide” refers to a polymeric form of nucleotides of any length, either ribonucleotides or deoxynucleotides, and includes both double- and single-stranded DNA and RNA. A polynucleotide may include nucleotide sequences having different functions, such as coding regions, and non-coding regions such as regulatory sequences (e.g., promoters or transcriptional terminators). A polynucleotide can be obtained directly from a natural source, or can be prepared with the aid of recombinant, enzymatic, or chemical techniques. A polynucleotide can be linear or circular in topology. A polynucleotide can be, for example, a portion of a vector, such as an expression or cloning vector, or a fragment.

As used herein, the term “polypeptide” refers broadly to a polymer of two or more amino acids joined together by peptide bonds. The term “polypeptide” also includes molecules which contain more than one polypeptide joined by a disulfide bond, or complexes of polypeptides that are joined together, covalently or noncovalently, as multimers (e g., dimers, tetramers). Thus, the terms peptide, oligopeptide, and protein are all included within the definition of polypeptide and these terms are used interchangeably, within context. It should be understood that these terms do not connote a specific length of a polymer of amino acids, nor are they intended to imply or distinguish whether the polypeptide is produced using recombinant techniques, chemical or enzymatic synthesis, or is naturally occurring.

The term “single-chain dimer” refers to a normally dimeric protein whose two subunits of coat polypeptide of a RNA bacteriophage have been genetically (chemically, through covalent bonds) fused into a single polypeptide chain. Specifically, in the present invention single-chain versions of bacteriophage often are constructed. Each of these proteins is naturally a dimer of identical polypeptide chains. In certain of the bacteriophages coat protein dimers of the N-terminus of one subunit lies in close physical proximity to the C-terminus of the companion subunit. Single-chain coat protein dimers may be produced using recombinant DNA methods by duplicating the DNA coding sequence of the coat proteins and then fusing them to one another in tail to head fashion. The result is a single polypeptide chain in which the coat protein amino acid appears twice, with the C-terminus of the upstream copy covalently fused to the N-terminus of the downstream copy. Normally (i.e., in wild-type) the two subunits are associated only through noncovalent interactions between the two chains. In the single-chain dimer these noncovalent interactions are maintained, but the two subunits have additionally been covalently tethered to one another. This greatly stabilizes the folded structure of the protein and confers to it its high tolerance of peptide insertions as described above.

In embodiments, the coat polypeptide of the VLP, often Qbeta or AP205 coat polypeptide, most often Qbeta, is prepared as monomeric units and the bacteriophage coat polypeptides self-assemble into VLPs typically comprising 180 copies of the coat polypeptide for each VLP.

The term “coding sequence” is defined herein as a portion of a nucleic acid sequence which directly specifies the amino acid sequence of its protein product. The boundaries of the coding sequence are generally determined by a ribosome binding site (prokaryotes) or by the ATG start codon (eukaryotes) located just upstream of the open reading frame at the 5′-end of the mRNA and a transcription terminator sequence located just downstream of the open reading frame at the 3′-end of the mRNA. A coding sequence can include, but is not limited to, DNA, cDNA, and recombinant nucleic acid sequences.

A “heterologous” region of a recombinant cell is an identifiable segment of nucleic acid within a larger nucleic acid molecule that is not found in association with the larger molecule in nature.

An “origin of replication” refers to those DNA sequences that participate in DNA synthesis.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation, as well as protein binding domains (consensus sequences) responsible for the binding of RNA polymerase. Eukaryotic promoters will often, but not always, contain “TATA” boxes and “CAT” boxes. Prokaryotic promoters contain Shine-Dalgamo sequences in addition to the −10 and −35 consensus sequences.

In bacteria, transcription normally terminates at specific transcription termination sequences, which typically are categorized as rho-dependent and rho-independent (or intrinsic) terminators, depending on whether they require the action of the bacterial rho-factor for their activity. These terminators specify the sites at which RNA polymerase is caused to stop its transcription activity, and thus they largely define the 3′-ends of the RNAs, although sometimes subsequent action of ribonucleases further trims the RNA.

An “expression control sequence” is a DNA sequence that controls and regulates the transcription and translation of another DNA sequence. A coding sequence is “under the control” of transcriptional and translational control sequences in a cell when RNA polymerase transcribes the coding sequence into mRNA, which is then translated into the protein encoded by the coding sequence. Transcriptional and translational control sequences are DNA regulatory sequences, such as promoters, enhancers, polyadenylation signals, terminators, and the like, that provide for the expression of a coding sequence in a host cell.

An “antibiotic resistance gene” refers to a gene that encodes a protein that renders a bacterium resistant to a given antibiotic. For example, the kanamycin resistance gene directs the synthesis of a phosphotransferase that modifies and inactivates the drug. The presence on plasmids of a kanamycin resistance gene provides a mechanism to select for the presence of the plasmid within transformed bacteria. Similarly, a chloramphenicol resistance gene allows bacteria to grow in the presence of the drug by producing an acetyltransferase enzyme that inactivates the antibiotic through acetylation.

The term “PCR” refers to the polymerase chain reaction, a technique used for the amplification of specific DNA sequences in vitro. The term “PCR primer” refers to DNA sequences (usually synthetic oligonucleotides) able to anneal to a target DNA, thus allowing a DNA polymerase (e.g. Taq DNA polymerase) to initiate DNA synthesis. Pairs of PCR primers are used in the polymerase chain reaction to initiate DNA synthesis on each of the two strands of a DNA and to thus amplify the DNA segment between two primers.

A cell has been “transformed” by exogenous or heterologous DNA when such DNA has been introduced inside the cell. The transforming DNA may or may not be integrated (covalently linked) into chromosomal DNA making up the genome of the cell. In prokaryotes, yeast, and mammalian cells for example, the transforming DNA may be maintained on an episomal element such as a plasmid, which normally replicate independently of the bacterial chromosome by virtue of the presence on the plasmid of a replication origin. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA.

A “signal sequence” can be included before the coding sequence. This sequence encodes a signal peptide, N-terminal to the polypeptide, that communicates to the host cell to direct the polypeptide to the cell surface or secrete the polypeptide into the media, and this signal peptide is clipped off by the host cell before the protein leaves the cell. Signal sequences can be found associated with a variety of proteins native to prokaryotes and eukaryotes.

It should be appreciated that also within the scope of the present invention are nucleic acid sequences encoding the polypeptide(s) of the present invention, which code for a polypeptide having the same amino acid sequence as the sequences disclosed herein, but which are degenerate to the nucleic acids disclosed herein. By “degenerate to” is meant that a different three-letter codon is used to specify a particular amino acid.

A nucleic acid molecule is “operatively linked” to, or “operably associated with”, an expression control sequence when the expression control sequence controls and regulates the transcription and translation of nucleic acid sequence. The term “operatively linked” includes having an appropriate start signal (e.g., ATG) in front of the nucleic acid sequence to be expressed and maintaining the correct reading frame to permit expression of the nucleic acid sequence under the control of the expression control sequence and production of the desired product encoded by the nucleic acid sequence. If a gene that one desires to insert into a recombinant DNA molecule does not contain an appropriate start signal, such a start signal can be inserted in front of the gene.

### Approach to the Present Invention (with Reference to First Set of References)

The four Dengue virus serotypes (DENV1-4) are mosquito-borne viruses that infect over 390 million people worldwide annually, primarily in developing nations. There are currently no antiviral treatments for DENV and prevention efforts rely on local control of the vector Aedes aegypti mosquito populations. Although efforts to develop a DENV vaccine have been pursued for almost 90 years, a safe and effective vaccine remains elusive largely due to the unique pathogenic features of DENV infection. Recent concerns regarding the safety of Dengvaxia (Sanofi Pasteur), specifically the increased risk of Severe Dengue in vaccinated individuals, have highlighted the need for novel vaccine strategies for DENV (references). DENV Non-Structural Protein 1 (NS1) has emerged as a promising target for vaccines that could prevent the most severe pathogenic sequelae of DENV infection (1, 2). NS1 is produced early in infection and is secreted in large quantities from infected cells. Circulating NS1 can cause plasma leakage by direct interaction with endothelial cells and also indirectly by activating immune cells to produce cytokines that cause further plasma leakage (3-8). Importantly, antibodies against NS1 can protect mice from lethal DENV infection (9-12) and NS1-mediated vascular leakage (11). However, some antibodies produced against NS1 have cross-reactivity with proteins on endothelial cells, which are hypothesized to further contribute to pathogenesis in the host (13-18). For this reason, NS1 is a promising target for a DENV vaccine, but for safety reasons care must be taken to avoid eliciting these harmful auto-reactive antibodies.

### Two Major Obstacles Exist in Pursuing NS1 as a DENV Vaccine Candidate:

[1] eliciting high titer, long-lasting antibodies to a subunit vaccine is challenging, and
[2] immunization with full-length NS1 can elicit pathogenic cross-reactive antibodies to human proteins (13-18).

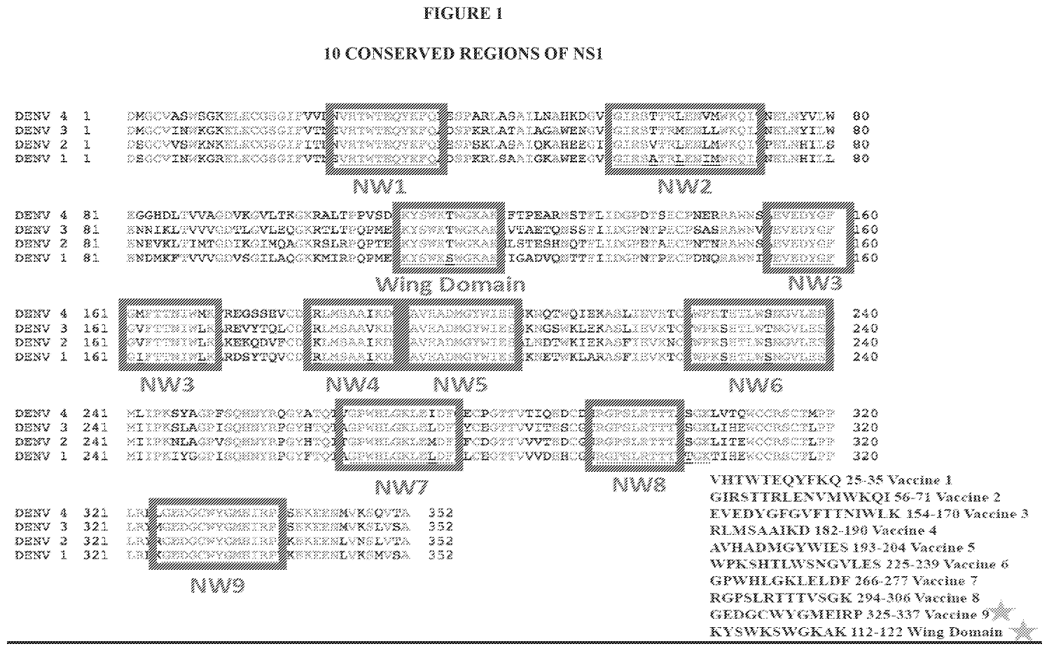

To overcome these challenges the inventors focused the use of a bacteriophage Qβ virus-like particle (Qβ-VLP) platform to elicit high titer, long-lasting antibodies to specific NS1 peptide epitopes. They have already successfully generated ten Qβ VLPs displaying different conserved regions of the NS1 protein (Qβ-NS1PEPs). See FIG. 1. In this proposal, these immunogens will be tested for their ability to elicit antibody responses in mice that block NS1-mediated endothelial cell tight junction disruption and cytokine release from peripheral blood mononuclear cells (PMBCs) in cell-culture, and protect against NS1-mediated vascular leak in vivo, but do not cross-react with human proteins on endothelial cells. The overall premise of our approach is supported by published data showing that bacteriophage VLP-based immunogens can elicit high titer, long-lasting antibody responses (19)(add more references), and data supporting NS1 as a promising vaccine candidate against DENV (references). The present approach allows for the fine-tuning of the antibody responses such that harmful cross-reactive epitopes can be avoided. This is in contrast to NS1 vaccine approaches that use full-length recombinant versions of NS1 as immunogens, where both NS1-blocking and host-protein cross-reactive antibodies are elicited.

### Determining the Binding Activity of Antibodies Elicited by Qβ-NS1PEP Immunogens.

The inventors immunize mice with Qβ-NS1PEP immunogens described above in order to elicit antibodies to the peptides of interest. Then, vaccines are identified that elicit antibodies that: [1] bind to DENV NS1, and [2] do not cross-react with human endothelial cell proteins. These experiments identify epitopes that are most suitable for an NS1 epitope-based vaccine.

Assessing the ability of Qβ-NS1PEP-elicited antibodies to block NS1-mediated mechanisms of DENV pathogenesis. The inventors assess serum antibodies from mice immunized with Qβ-NS1PEP immunogens for their ability to block: [1] NS1-mediated tight-junction protein disruption of vascular endothelial cells, and [2] NS1-mediated cytokine production by peripheral blood mononuclear cells (PBMCs).

In vivo assessment of lead vaccine candidates to protect against NS1-mediated vascular leakage. Vaccine candidates will be identified from Aim 1 and Aim 2 based on their ability to elicit antibodies that block NS1-mediate activity but not bind to human endothelial cell proteins. These lead candidates will be tested in an established mouse model of NS1-mediated vascular leakage.

The present invention utilizes a highly immunogenic, multivalent vaccine platform to elicit targeted antibody responses to NS1 protein as an innovative strategy to develop new DENV vaccines. The primary objective in this “high-risk/high-reward” pre-clinical development project is to identify lead vaccine candidates to move into more extensive safety and efficacy testing. This work also provides insight into the role of specific antibody responses to NS1 during natural DENV infection.

The need for novel Dengue Virus Vaccines. DENV is transmitted by the Aedes aegypti mosquito and causes large outbreaks that are difficult to manage for resource poor communities. Although over half of the world's population is at risk for DENV infection, there are currently no antiviral treatments, and the only licensed vaccine, Dengvaxia (Sanofi-Pasteur), has recently been pulled from population-based vaccination campaigns because of serious safety concerns (20).

The unique features of DENV pathogenesis make generating a safe vaccine especially challenging. DENV is composed of four serotypes (DENV-1, -2, -3, and -4). Infection with one serotype (primary infection) results in long-lasting immunity to that serotype and the production of serotype-specific neutralizing antibodies (21). A subsequent infection with another serotype (secondary infection) carries an increased risk of a cluster of adverse sequelae known as Severe Dengue, including Dengue Hemorrhagic Fever and Dengue Shock Syndrome. These severe sequelae are characterized by plasma leakage, hemorrhage, shock, and cytokine storm (22). The increased risk of Severe Dengue upon secondary infection is thought to be the result of a phenomenon termed “Antibody Dependent Enhancement of Infection” (ADE) whereby antibodies elicited in response to primary infection bind to, but do not block the infection of other DENV serotypes (23). Upon infection with a heterologous serotype later in life, these non-neutralizing antibodies facilitate entry of the DENV into immune cells and increase viral load in the patient, resulting in Severe Dengue. This risk of ADE has directed prior art vaccine efforts toward simultaneously eliciting broadly neutralizing antibody responses to all four DENV serotypes, by using tetravalent vaccine preparations. Dengvaxia is based on this approach, but the vaccine was not able to elicit comparable responses to all four DENV serotypes, resulting in an increased risk of Severe Dengue in vaccinated individuals.

NS1 as a target for vaccines according to the present invention. NS1 is a central mediator of DENV pathogenesis. Vascular leakage leading to hemorrhage and shock is a key pathogenic feature of Severe Dengue. Recent data have implicated the DENV protein NS1 in Severe Dengue manifestations (24). As a non-structural protein, NS1 is not a component of the virion, but rather is a protein produced from the viral genome in infected cells that is involved in other aspects of the virus-host interactions. NS1 is produced early during infection before viral replication occurs and is secreted in large quantities from infected cells into the blood (2). The detection of high serum levels of NS1 (>600 ng/mL) within the first 72 hours of illness is associated with Dengue Hemorrhagic Fever (3). NS1 is thought to cause plasma leakage by disrupting vascular endothelial cell tight junctions by two proposed mechanisms: [1] NS1 binds to Toll-like receptors (TLRs) on PBMCs, stimulating the production of cytokines (IL-10, IL-6, and TNF-α) that damage the vascular endothelial cell integrity, and [2] directly binding to vascular endothelial cells and causing the disruption of tight junctions through an as-yet-undefined mechanism (2). Taken together, these data provide evidence that excessive NS1 production may drive plasma leakage. For this reason, NS1 has recently become a promising target for vaccine efforts. NS1 vaccines would protect against NS1-mediated pathogenesis, but would not be expected to provide sterilizing immunity against viral infection. However, these efforts are complicated by data showing that a subset of antibodies against NS1 can themselves cause damage to endothelial cells through cross-reactivity to host proteins on the endothelial cells (25, 26). Because of this, vaccines against DENV NS1 protein need to elicit antibodies that block NS1 activity but not cross-react with and cause damage to endothelial cells. The immunodominant epitope of NS1 in humans and mice is the wing-domain, a disordered region of the protein (27, 28). Importantly, the wing-domain contains a cross-reactive motif, KXWG (25), and antibodies to the wing-domain cross-react with host proteins (13, 14, 25). Other regions of NS1 can also elicit cross-reactive antibodies to platelets and coagulation factors (17, 29). These antibodies have been shown in vitro and in vivo to have pathogenic activity (17, 18). Pursuant to the present invention, epitope-specific vaccines against NS1 are engineered to avoid the production of these harmful, cross-reactive antibodies while providing protection from the NS1-mediated pathogenic mechanisms. The present invention is able to achieve this specificity while still eliciting high titer, long-lasting antibody responses-something that is difficult to do with KLH or other protein carrier approaches often used for epitope-based vaccines.

Previous Approaches to providing NS1 vaccine. Immunogen choice for previous NS1 vaccination efforts fall into 3 categories: full-length recombinant NS1 (9, 11, 30-33), subunit-peptide immunogens conjugated to a carrier protein (i.e. KLH) (12), and DNA vaccines (34-38). All of these efforts, except the subunit-peptide immunogen strategy, elicit polyclonal antibody responses against multiple epitopes of NS1, but predominantly the immunodominant wing-domain (references). As such, these strategies have the possibility of eliciting antibody responses against cross-reactive epitopes. Indeed, mice immunized with full-length recombinant NS1 produced antibodies that recognize the wing-domain (28). The only published subunit-peptide immunogen design is based on KLH-conjugation of a peptide corresponding to the NS1 wing-domain modified to alter the KXWG cross-reactive epitope (12). Passive-transfer of concentrated, purified polyclonal antibodies from mice immunized with this modified NS1 wing-domain peptide protected against lethal DENV challenge. However, immunization with a subunit vaccine conjugated to KLH or other non-multivalent carrier proteins require multiple boosts and exogenous adjuvants to reach sufficiently high titers, and circulating antibody responses are often short-lived. For these reasons, subunit vaccines such as the above-described may not be appropriate for delivery to populations most in need of DENV vaccines (i.e. resource poor countries in tropical and sub-tropical regions of the world).

### Use of Bacteriophage VLPs for DENV Vaccines of the Present Invention.

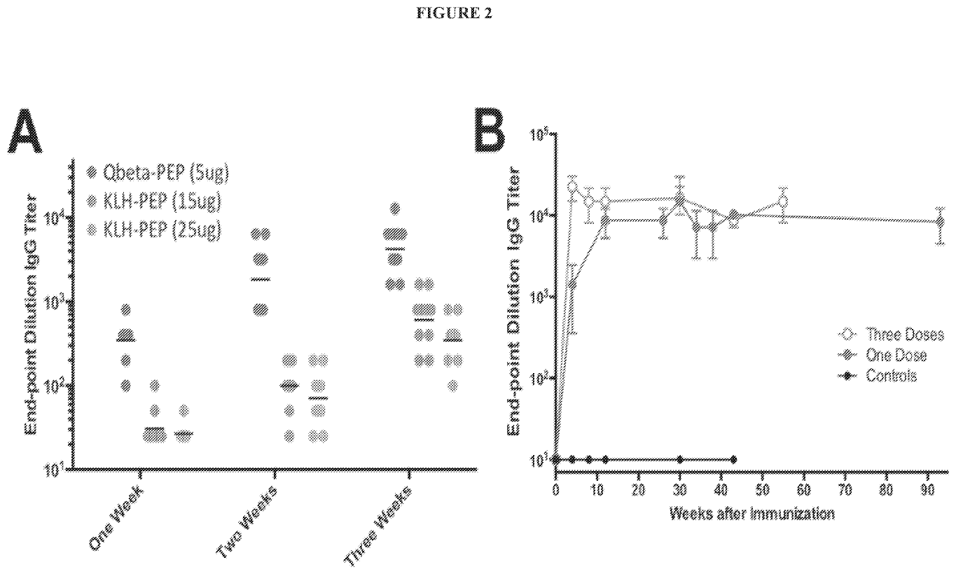

Bacteriophage VLPs are highly immunogenic vaccine platforms that readily elicit high titer, long-lasting antibodies to short peptide antigens. VLPs are multivalent platforms that can be used to dramatically increase the immunogenicity of molecules that are normally poorly immunogenic (such as short peptides). FIG. 2 illustrates the power of VLP display by showing a comparison of the IgG titers elicited in response to a short peptide when displayed on Qβ VLPs vs. KLH. A single dose of the VLP vaccine elicits anti-peptide antibodies as early as a week after immunization, and high titer responses by 2-3 weeks. In contrast, antibody responses to the KLH-conjugated peptide are slower to develop and reach lower titers. Moreover, VLPs elicit long-lived antibody responses. In FIG. 2B, a single immunization with a VLP-based vaccine leads to antibodies that last for the lifetime of the mouse. The only other published effort at generating a epitope-specific, peptide-based NS1 vaccine relied on KLH-conjugation to increase immunogenicity (12). KLH-conjugation strategies require exogenous adjuvants and multiple boosts in order to effectively elicit antibodies. The inventors proposed that an alternative approach, using Qβ VLPs, might elicit high-titer and long-lasting antibodies more rapidly, potentially after only one dose, and without the use of exogenous adjuvants.

Addressing safety and feasibility concerns regarding NS1 vaccination. NS1 is a promising vaccine target for the prevention of Severe Dengue, but there are both feasibility and safety concerns with using full-length NS1 as a vaccine, which is the strategy currently being employed by other groups (28, 32, 33). First, it is difficult to generate long-lasting, high titer antibody responses to a vaccine based on a recombinant protein. Recombinant protein vaccines generally require multiple boosts in order to effectively provide protection. Because the primary target populations for a DENV vaccine are in developing countries of Central and South America and Southeast Asia, the feasibility of administering multiple doses is problematic. Additionally, some antibodies elicited in response to NS1 are cross-reactive to host proteins and are thought to contribute to the pathogenesis of DENV secondary infection (1). Immunization with the full NS1 protein would likely elicit both protective and harmful antibody responses: indeed, in natural infection the immunodominant epitope, known as the “wing domain” is also the target of cross-reactive antibodies (13-15, 17, 18) and immunization of mice with full-length NS1 similarly results in antibodies against the wing-domain (28). In contrast, the present invention provides an innovative way to dissect the functions of the epitope-specific antibody responses. By eliciting antibodies only to specific NS1 peptides, the present invention focuses the vaccine-elicited antibody response only to those epitopes that prevent NS1 pathogenic activity, while avoiding harmful cross-reactive antibodies. Thus, in contrast to previous approaches, the present invention focuses on regions of NS1 protein that are conserved among the four DENV serotypes and that do not include previously identified cross-reactive epitope-ELK/KLE and KXWG epitopes. These are identified above and are also set forth in FIG. 1. Conserved regions NS1 are likely to be important for the function and or structural integrity of the protein, so binding of antibodies to these regions may block critical NS1 functions. Sequence homology does not necessarily predict cross-reactivity, so empirically assessing the antibodies elicited by our vaccines for cross-reactivity to endothelial cells will be important. By using a VLP as the vaccine platform, the present invention provides several important benefits that are likely to improve the feasibility of delivering vaccine to those most in need. Because they often are made in E. coli, bacteriophage VLP vaccines are expected to be relatively inexpensive to manufacture. Additionally, the safety and immunogenicity of Qβ VLP-based vaccines has been established in several clinical trials (41-44). Bacteriophage VLPs are thermostable, suggesting that a cold chain may not be necessary to deliver VLP-based vaccine to areas in need (19, 45). Finally, there is evidence that a single immunization with VLPs may be sufficient to elicit protective antibody responses (45). These unique features of VLP vaccines make this platform an attractive approach for DENV NS1 vaccine design.

### Construction of Qβ-NS1PEP Immunogens

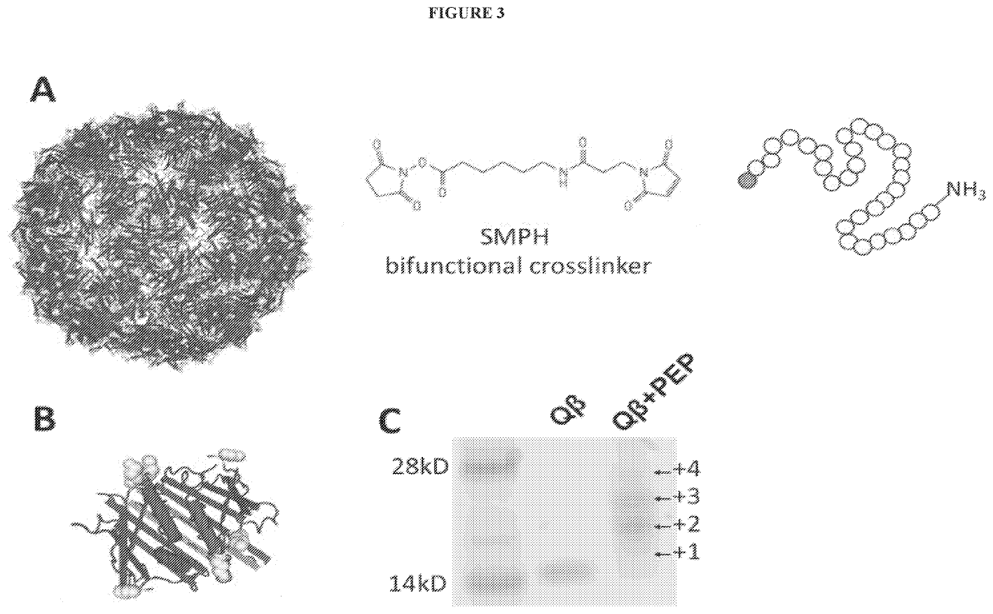

In choosing NS1 peptide epitopes to investigate, the inventors considered the conservation of the NS1 protein sequence among the four DENV serotypes as well as the lack of known cross-reactive epitopes (ELK/KLE and KXWG). This resulted in 8 peptide sequences of interest that have been investigated as vaccine candidates. Additionally included were two known cross-reactive epitopes (ELK/KLE epitope corresponding to amino acids 266-277 and KXWG Wing Domain, corresponding to amino acids 112-122 of NS1 peptide) to use as positive controls HUVEC binding assays. Also included was a modified version of the Wing Domain which replaces the KS of the KXWG epitope with SE. This modification of the Wing Domain peptide has been shown to elicit antibodies that show beneficial activity in blocking NS1 but do not cross-react with host proteins. These peptides were synthesized with a C-terminal tri-glycine linker sequence and a cysteine (containing a free sulfhydryl group) to allow chemical conjugation to the Qβ VLPs with Succinimidyl 6-((beta-maleimidopropionamido)hexanoate) (SMPH). Chemical conjugation of the peptides to Qβ VLPs was carried out by reacting SMPH with surface exposed lysines on the VLPs followed by addition of NS1 peptide at a 10-fold molar excess (FIG. 3). Conjugation of the peptides to the Qβ VLPs was then confirmed by SDS-PAGE, an example of which is shown in FIG. 3C.

### DENV-Infected Patients Generate Antibodies to NS1 Peptides

In a parallel project, the inventors investigated the specificity of the antibody response to DENV in infected humans as a way to identify vaccine candidates and diagnostic and prognostic tests. Using a novel method developed and called Deep Sequence-Coupled Biopanning, the inventors investigated the DENV antibody specificity of serum from patients with secondary DENV infection. The peptide KYSWKSWGKAK (SEQ ID NO: 10 amino acids 112-122 of NS1, Cross-reactive) as a major immunodominant epitope in a patient with acute secondary DENV infection (46). Using enzyme-linked immunosorbent assays (ELISA), it was shown that many patients with primary and secondary DENV infection have antibodies to the KYSWKSWGKAK peptide (SEQ ID NO: 10) (46). This is intriguing, as it suggests that anti-NS1 antibodies are common among patients. Indeed, others have identified this region of NS1, referred to as the Wing Domain due to its location in the structure of NS1, as an immunodominant epitope of NS1. Interestingly, the KYSWKSWGKAK peptide includes the cross-reactive epitope (KXWG), which has been shown to be present on LYRIC proteins on the surface of HUVEC cells (25). In another study, the NS1 antibodies that cross-react with host proteins on endothelial cells may be important in the pathogenesis of DENV. The inventors included the KYSWKSWGKAK peptide in the panel of vaccine candidates as a positive control for a vaccine that elicits antibodies that cross-react with host proteins on endothelial cells. The inventors also have preliminary data from another Deep Sequence-Coupled Biopanning experiment with DENV patient serum that shows common antibody responses to peptide GEDGCWYGMEIRP, SEQ ID NO:8 (Data not shown).

### Characterizing the Binding Activity of Antibodies Elicited by Qβ-NS1PEP Immunogens.

Identification of Qβ-NS1PEP immunogens which elicit antibodies that (1) bind to recombinant NS1 protein from the 4 DENV serotypes, but (2) do not cross-react with proteins expressed on endothelial cells is performed. In these studies, groups of mice (n=6, 3 male and 3 female) are immunized with the 10 VLP-based vaccines. Mice are immunized 3 times at 3-week intervals with 5 μg VLP/immunization/mouse by intramuscular immunization in the right hind leg. This immunization strategy assures the highest titers of antibodies in the serum. Three weeks after the final immunization, mice are sacrificed and serum is collected by cardiac puncture. Serum is then used in subsequent experiments.

Assessing the Ability of Vaccine-Elicited Antibodies to Bind to NS1 Protein from the 4 DENV Serotypes.

It was hypothesized that antibodies elicited by the vaccines will bind to NS1 proteins from all 4 DENV serotypes. The peptides chosen (SEQ ID Nos:1-10, above and FIG. 1) was based on their conservation among the 4 DENV serotypes. SEQ ID NO:10 was chosen based upon a modification of amino acids 112-122 of NS1 by substitution of SE for KS in the native NS1 peptide SEQ ID NO:11 (SEQ ID NO:10 shown not to cross react, unlike native peptide SEQ ID NO:11). This hypothesis is tested by carrying out ELISA against recombinant NS1 obtained from a commercial source (The Native Antigen Company, Oxford, UK). This source of purified and correctly folded NS1 is widely used within the DENV field (4, 5, 47). To assure robust and unbiased data, each mouse serum sample is assessed in triplicate by ELISA against each of the NS1 proteins and the technician performing the ELISA is blinded to the identity of the samples.

### Assessing the Ability of Vaccine-Elicited Antibodies to Bind to Human Endothelial Cells.

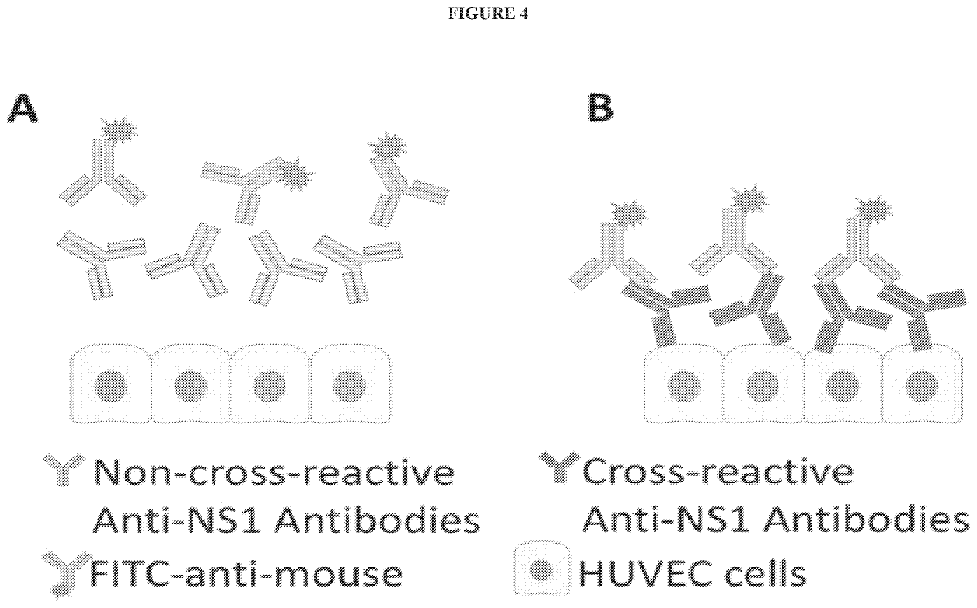

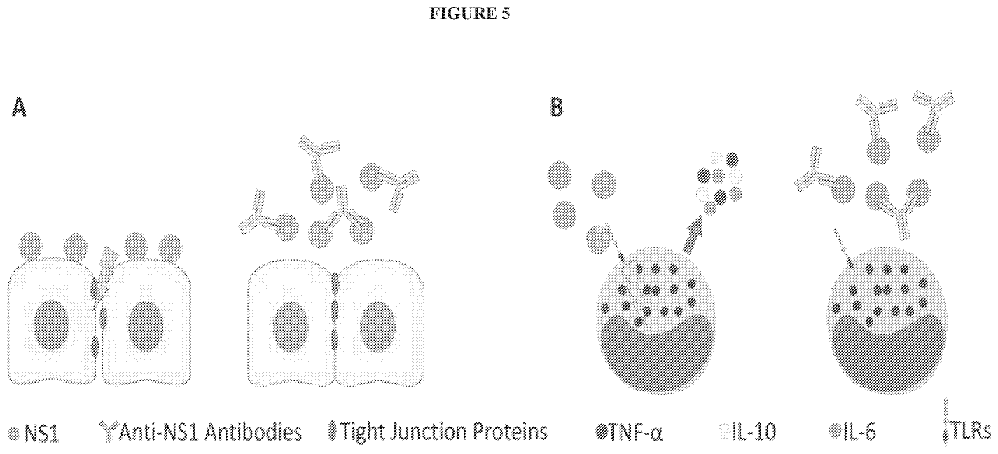

One of the major safety concerns of using NS1 as a vaccine antigen is that it is capable of eliciting antibodies that cross-react with host proteins on vascular endothelial cells, resulting in complement-mediated damage to vascular endothelial cells, leading to vascular damage and leakage (15, 48). In these experiments, antibodies elicited by the vaccines are assessed to see if they exhibit this dangerous binding phenotype. Assessment of vaccine-elicited antibodies for binding to HUVEC endothelial cells by confocal microscopy is performed in order to screen widely for cross-reactivity of the vaccine-elicited antibodies to endothelial cell proteins (FIG. 4). HUVEC cells express LYRIC protein (a host protein for which some antibodies to NS1 cross-react) as well as other auto-antigens to which NS1-antibodies bind (26, 49). HUVEC cells are plated on coverslips and grown to confluency. Cells are fixed in 2% paraformaldehyde, washed in PBS, and dilutions of sera from vaccinated mice are incubated on the coverslips. Bound antibodies from the sera are detected by FITC-conjugated anti-mouse secondary antibody and confocal microscopy. Sera from mice vaccinated with the KYSWKSWGKAK peptide (SEQ ID NO: 11, which is known to elicit cross-reactive antibodies to LYRIC protein) serve as a positive control for these experiments and sera from mice immunized with Qβ VLPs alone (not conjugated to peptide) serve as a negative control. Additionally, assessment of serum for binding to LYRIC protein specifically by Western blot is performed. To assure robust and unbiased data, microscopy slides are coded for staining and each sample is assessed by microscopy in triplicate. Positive staining is reported as the dilution at which signal is observed above that observed with negative control (sera from mice immunized with Qβ VLPs alone).

The ability of antibodies to block the activity of NS1 in cell culture-based assays provides essential information on each of the candidate vaccines that will be utilized in order to down-select candidates to test in in vivo studies. An ideal vaccine candidate elicits high-titer antibodies that bind to NS1 proteins from all four DENV serotypes and do not show binding to HUVEC cells. The preferred candidate peptides of the present invention were chosen primarily because of their conservation among the four DENV serotypes. However, there is variation in efficiency of serum binding among the NS1 proteins of the four DENV serotypes. This is taken into consideration when choosing lead vaccine candidates. Previous research has shown that some antibodies elicited by NS1 vaccination are capable of binding to HUVEC cells by microscopy (14, 25), so this is the current gold-standard for identifying cross-reactive antibodies to human proteins elicited by NS1.

### Assessing the Ability of Qβ-NS1PEP-Elicited Antibodies to Block NS1-Mediated Mechanisms of DENV Pathogenesis.

The activity of vaccine elicited antibodies to block two key mechanisms of action of NS1 in causing vascular leakage-[1] disruption of tight junctions of vascular endothelial cells and, [2] production of IL-10, IL-6, TNF-α by PBMCs is assessed. Both of these mechanisms require direct binding of NS1 to the target cells (endothelial cells or PBMCs). Vascular leakage is a major pathogenic feature of dengue virus infection, and NS1 is a key mediator of this process. A vaccine that elicits antibodies that block the mechanisms by which NS1 leads to vascular leakage could prevent Severe Dengue and save lives.

### Assessing the Ability of Vaccine-Elicited Antibodies to Block NS1-Mediated Disruption of Tight Junctions on Vascular Endothelial Cells.

These experiments utilize commercially available human pulmonary microvascular endothelial cells (HPMECs) (SciCell Research Laboratories, Inc.). When recombinant DENV NS1 is added to HPMECs, tight junctions are disrupted (FIG. 5A)(4). This disruption can be quantitatively assessed by measuring trans-endothelial electrical resistance (TEER). A previously described method for monitoring NS1-mediated disruptions of TEER (4) is used. Briefly, HPMECs are grown on a 24-well transwell polycarbonate membrane system (Corning). Cells are treated with recombinant NS1 (Native Antigen Company, Oxford, UK) pre-incubated with various dilutions of serum from mice immunized with vaccine candidates, with serum from mice immunized with control Qβ VLPs without conjugated peptide, no serum (positive control), or ovalbumin (negative control). TEER is measured in Ohms using an Epithelial Volt Ohm Meter (World Precision Instruments) with measurements taken sequentially every 2-hours after the addition of NS1 protein (or control). Relative TEER is a ratio of resistance values represented by the equation (Ohm experimental condition−Ohm medium alone)/(Ohm ovalbumin treated cells−Ohm medium alone). Vaccine-elicited antibodies that show positive results by TEER are confirmed by assessing NS1-mediated disruption of tight-junctions by confocal microscopy and Western blot. Cells are grown and treated with NS1 and serum as described for TEER measurements, but 13 hr after treatment (the peak of TEER loss in previous experiments, (4), transwells are processed for microscopy by staining for tight junction protein VE-cadherin (50) and for Western blot with staining for VE-cadherin. Vaccine-elicited sera that block the reduction in relative TEER induced by NS1 protein and prevent the loss of VE-cadherin from the inter-endothelial cell junctions are identified as positive vaccine candidates moving forward. To assure robust and unbiased data, the experiments are coded such that the technician is unaware of the source of the antibodies and each sample is assessed in independent experiments carried out on separate days by different technicians.

### Assessing the Ability of Vaccine-Elicited Antibodies to Block NS1-Mediated Cytokine Production in PBMC

Commercially available human PBMCs (Lonza, Inc.) and ELISAs are used to detect IL-10, IL-6, and TNF-α production in response to NS1. PBMCs are seeded at 50,000 cells/well in 96-well tissue culture plates and treated with NS1, NS1+sera from vaccinated mice, or LPS (positive control). Supernatant is collected at 24 hours post treatment and IL-10, IL-6, and TNF-α is measured by commercially available ELISAs (Fisher Scientific). Cytokine production is analyzed by comparing cell supernatants from NS1+sera to NS1. Vaccine-elicited antibodies that block the NS1-induce cytokine production in PBMCs is identified as positive vaccine candidates moving forward. To assure robust and unbiased data, the experiments are coded such that the technician is unaware of the source of the antibodies and each sample is assessed in independent experiments carried out on separate days by different technicians.

The ability of antibodies to block the activity of NS1 in these cell culture-based assays provides important information in the down-selection of candidates for in vivo experiments and also contribute to an understanding of the activity of specific epitopes of NS1. Ideal vaccine candidates selected block the ability of NS1 to disrupt endothelial cell tight junctions and NS1-mediated cytokine production. These are two well-established activities of NS1 that are likely to contribute to the pathogenic mechanisms of NS1. Perhaps NS1 has other important mechanisms of action that have yet to be discovered. However, blocking these two important mechanisms provides sufficient justification to pursue VLP-based candidates in the in vivo studies, described herein below.

### In Vivo Assessment of Lead Vaccine Candidates to Protect Against NS1-Mediated Vascular Leakage.

Experiments carried out as described herein above determine the selection of vaccine candidates that are tested in vivo. In order to be included in the in vivo assessment, antibodies elicited by the vaccine candidate must meet the following criteria: show binding to NS1 protein from at least one DENV serotype, show no binding to HUVEC cells or LYRIC protein, block NS1-mediated trans-endothelial cell permeability and NS1-mediated cytokine production in PBMCs. Groups of C57BL/6 mice (n=10, 5 males, 5 females) are immunized three times at 3-week intervals with the vaccines that meet the above criteria and Qβ-VLPs alone (negative control). Three weeks after the final immunization, serum is collected and assessed for antibody titer to the cognate peptide. Thereafter, assessment is performed of immunized mice for NS1-mediated vascular leakage with a dextran-adapted dermal Miles assay as described previously (4). Dorsal hair is removed 3-4 days prior to the experiment. On the day of the experiment, mice are anesthetized with isofluorane and each mouse is injected intradermal (ID) with 50 μL of PBS (negative control), VEGF (200 ng/50 μL PBS) (positive control), DENV2 NS1 (15 μg/50 μL PBS and 7.5 μg/50 μL PBS) into distinct sites into the shaved back skin. Immediately following ID injections, 200 μL of 1 mg/mL Alexa Fluor 680 conjugated 10 kDa dextran (Sigma) is administered by retro-orbital injection. Two hours post-injection, mice are euthanized and the dorsal dermis removed. Tissues are assessed for fluorescence at 700 nm on a LI-COR imaging system. Quantitation of fluorescent dextran leakage in a 13 mm diameter circle around the injection sites is carried out using imaging software and the output reading is mean pixel intensity. Protection from NS1 mediated vascular leak is identified by a significant decrease in the mean pixel intensity of mice immunize with candidate vaccines compared to mice immunized with Qβ-VLPs alone (negative control).

The initial assessment of the ability of lead vaccine candidates to block NS1-mediated vascular leak in vivo utilizes a high sensitivity fluorescent dextran model of vascular leakage. An alternative, but more traditional method to measure vascular leakage uses Evans Blue Dye and quantitation by formamide extraction (4). This technique is not as sensitive as the fluorescent dextran model, but could be employed as an alternative approach. Initially vaccine candidates are tested against NS1 from DENV2. If a vaccine candidate does not elicit antibodies that strongly bind to DENV2 NS1, a more appropriate DENV serotype is used. Initially, NS1 peptides are selected that are unlikely to elicit cross-reactive antibodies and include one peptide candidate that previously has been shown to elicit desirable antibodies. Identified peptides are tested on a bacteriophage VLP platform (Qβ bacteriophage) as described herein below.

### Further Experiments (with Reference to Second Set of References)

### Materials and Methods

### Peptide Sequences

The sequences of the entire DENV NS1 protein from all four serotypes (DENV-1-4) were aligned to identify sequence homology and areas of high conservation among the four serotypes. The accession numbers for the reference sequences of each of the four serotypes are as follows: DENV-1 (NP_722461.1), DENV-2 (NP_739584.2), DENV-3 (YP_001531169.2), and DENV-4 (NP_740318.1).

### Growth of Qβ VLPs

Q-Beta (Qβ) VLPs were made using similar methods as previously described [33,35,36]. Briefly, Escherichia co/i (E. co/i) C₄₁ cells (Sigma-Aldrich) were transfected with the plasmid pET containing the Qβ coat protein coding sequence under an IPTG-inducible promoter [31,32]. Cells were grown until OD₆₀₀ of 0.6 was reached. Cultures were then induced with 0.5 mM of Isopropyl β-d-1-thiogalactopyranosid (IPTG) for three hours and pelleted by centrifugation. Pellets were resuspended in a lysis buffer consisting of 50 mM Tris-HCl, 10 mM EDTA, and 100 mM NaCl. Cells were then incubated for 30 min on ice after the addition of deoxycholate (DOC) to a final concentration of 0.05%. Suspensions were sonicated for one minute intervals, five times and replaced on ice between sonication. 10 mg/mL of Dnase and 2 mM MgCl₂ was added to the solution and incubated at 37° C. for 1 h to digest residual bacterial DNA. Lysates were centrifuged and ammonium sulfate was added to the supernatant at a 60% saturation overnight. Ammonium sulfate/lysates were centrifuged at 10,000 RPM and pellets were resuspended in sepharose column buffer (SCB) containing 10 mM Tris-HCl, 0.1 M NaCl, and 2 mM MgSO₄ to a QS of 1 L deionized water. Suspensions were frozen at −80° C. until size-exclusion chromatography was performed. Samples were added to a chromatography column filled with Sepharose CL-48 beads (Sigma-Aldrich) in SCB and fractions containing Qβ VLPs were then identified via agarose gel electrophoresis and denaturing SDS-PAGE gels. Fractions were combined and Qβ VLPs were precipitated by adding 70% ammonium sulfate overnight. A buffer exchange was performed overnight using Snakeskin Dialysis Tubing 10 K molecular weight cutoff (Thermo Fisher Scientific) in phosphate buffered saline.

Qβ VLP stocks were then depleted of LPS using sequential Triton X-114 phase extraction. Triton X-114 was added to stocks at a final volume of 1%. Samples were vortexed, incubated on ice for five minutes, followed by a five minute incubation in a 37° C. heat block. Samples were spun at max speed for 1 min at 37° C. The aqueous phase was moved to a clean tube, and the process was repeated four more times for a total of five times. Concentrations of Qβ was determined by SDS-PAGE gel using known concentrations of hen's egg lysozyme as a comparison. Correct assembly of Qβ VLPs before and after is assured by buffer exchange and filtration through appropriate molecular weight cutoff such that non-assembled coat protein will not be present in final inoculum. Stocks were frozen at −20° C. until use.

### Conjugation of Peptides to Qβ VLPs

Peptides of interest were commercially synthesized by Genscript and reconstituted in the manufacturer's recommended solvent prior to use. Using the bifunctional crosslinker succinimidyl 6-((beta-maleimidopropionamido) hexanoate) (SMPH), peptides of interest were conjugated to the surface-exposed lysines of Qβ, through an added linker sequence of -GGGC at the C-terminal region. Peptides were added at a 10:1 ratio of peptide to Qβ stock. Excess SMPH and peptide were removed through Amicon filtration (Millipore Sigma).

### Immunizations

6-8-week-old BALB/c mice (Jackson Labs, male and female) were vaccinated intramuscularly in the hind leg twice, at three-week intervals, with 5 μg/50 μL of vaccine with no exogenous adjuvant. Retro-orbital bleeds were performed three weeks after first immunization (D21) and three weeks after second immunization (D42) to collect sera for ELISAs. Animals were sacrificed upon verification of antibody titers via cardiac puncture. All animal studies were performed in accordance with guidelines of the University of New Mexico Animal Care and Use Committee (Protocol #: 20-201021-HSC).

### ELISA

Cognate peptides: Synthetic peptide ELISA was performed as previously described [37]. Briefly, Immunolon 96-well ELISA plates were coated with 1 μg/100 μL of Streptavidin (Invitrogen) in PBS at 4° C. overnight. Plates were washed three times with PBS and incubated for one hour at room temp with 2 μg/100 μL of SMPH. Plates were washed with PBS three times and peptides correlating with immunizations were plated in 100 μL volumes at 0.02 μg/μL and incubated for two hours at room temperature. Plates were washed three times with PBS and blocked over night with 100 μL of 0.5% milk in PBS. Plates were washed twice, and mouse sera were diluted in 0.5% milk/PBS in four-fold dilutions starting with 1:40 and ending with 1:655,360. Plates were washed five times and goat anti-mouse conjugated with horseradish peroxidase (HRP) secondary antibody (Jackson ImmunoResearch) was added at 1:5000 dilution in 50 μL volumes to each well for 45 min. After washing plates 5 times, 50 μL of soluble TMB (Millipore Corp.) was added to each well. Plates were incubated for 15 min and quenched with 50 μL of 1% HCl solution. Absorbance at 450 nm was determined.

### Soluble DENV NS1

ELISA against DENV NS1 was performed as follows. A volume of 0.16 μg/well of soluble NS1 protein produced in HEK 293 cells (The Native Antigen Company, Oxford, United Kingdom) in 50 μL was added to Immunolon 96-well ELISA plates overnight. Plates were washed three times with PBS and blocked for 2 h at room temperature with 100 μL of 0.5% milk in PBS. Plates were washed 3 times and sera from immunized mice were added in 50 μL volumes and serially diluted starting with a 1:40 dilution and 4-fold dilutions were tested up to 1:655,360. Plates were incubated for 2 h at room temperature with rocking, followed by PBS wash of 5 times. A volume of 50 μL/well of 1:5000 dilution of goat anti-mouse secondary antibody conjugated with HRP (Jackson ImmunoResearch) was added to each well. Plates were washed 5 times with PBS, followed by the addition of 50 μL of TMB for 15 min, and quenched with 50 μL of 1% HCl. Absorbance at 450 nm was determined.

### Cell Culture and Virus Stocks

Human embryonic kidney 293 (HEK 293) cells were purchased from American Type Culture Collection (ATCC, CRL-1573). Cells were grown in complete growth medium consisting of ATCC-formulated Eagle's Minimum Essential Medium (EMEM, cat no. 30-2003) supplemented with a final concentration of 10% fetal bovine serum (FBS).

DENV-2 New Guinea C (NGC), kindly provided by Dr. Kathryn Hanley at New Mexico State University (NMSU), was cultured in C6/36 cells (ATCC) to produce working viral stocks. Virus was collected in 1× SPG consisting of 2.18 M sucrose, 38 mM potassium phosphate (monobasic), 72 mM potassium phosphate (dibasic), and 60 mM L-glutamic acid. Samples were clarified by centrifugation and stored at −80° C.

### Antibody Binding to DENV-Infected Cells

HEK293 cells were plated in 96-well glass bottom plates (Cellvis) at 25,000 cells per well overnight. Cells were washed once with complete HEK media. Cells were infected at an MOI of 100 PFU/well with DENV-2 NGC diluted in complete HEK media for 20 min to allow for binding to cells. 150 μL of HEK media was added to wells and plates were incubated for 72 h at 37° C. Cells were washed 1 time with PBS and fixed two times for 20 min using 200 μL of 100% methanol. Methanol was removed and cells were washed 2 times with PBS and blocked with 100 μL of 2% goat sera (Jackson ImmunoResearch) in PBS for 1 hour at room temperature. Mouse sera were diluted in 2% goat sera/PBS containing a 1:1000 dilution of rabbit anti-DENV 4G2 antibody (The Native Antigen Company). As a positive control, some cells were instead stained with anti-DENV NS1 antibody (mouse monocloncal, Flavivirus NS1 (D/2/D6/B7) (Abcam, 214,337) at a 1:500 dilution. Cells were treated with 50 μL volumes at two-fold dilutions of sera starting with 1:40 going up to 1:320 for 1 hour at room temperature with rocking. Plates were washed three times with PBS. Plates were incubated with 50 μL of Alexa fluor 488 goat anti-mouse IgG (Abcam; 1:1600), Alexa fluor 647 goat anti-rabbit IgG (Jackson ImmunoResearcher; 1:1000), and Hoechst (Thermo-Fisher; 1:2000) for 1 hour at room temperature. Plates were washed 3 times and 150 μL of PBS was replenished to each well. This research made use of the Fluorescence Microscopy and Cell Imaging Shared Resources which is partially supported by UNM Comprehensive Cancer Center Support Grant. Plates were imaged on a Zeiss Axio Observer epifluorescence microscope with a Hamamatsu Flash 4.0 camera using Slidebook imaging software.

### Results

Identification of Conserved Epitopes of NS1 to Target with Vaccination

The DENV NS1 amino acid sequence of all four DENV serotypes were aligned in order to identify highly conserved 9-17 amino acid regions of the NS1 proteins (See FIG. 1). Our goal is to develop a vaccine candidate that is highly conserved and protective among the four serotypes of DENV. Although most of the vaccine candidates were of highly conserved regions of the NS1 protein where no differences in amino acid sequences were observed (red), we used the sequences from either DENV-2 or DENV-3 for each vaccine candidate. Among the highly conserved regions of NS1 were several regions of interest. The first was aa 112-122 which is the immunodominant region of NS1 wing domain [18,38]. This region of NS1 has been highly associated with antibodies that strongly bind to NS1, but can also have cross-reactive effects in binding to the surface of endothelial cells and inducing damage to these cells [39-48]. Another region of interest was that of aa 325-337. DENV-infected patients make antibodies to this region of NS1, and these antibodies have been implicated in the binding of platelets and impacting coagulation pathways [47,49]. Interestingly, we have identified antibodies against a number of other highly conserved sequences selected for this study in DENV-infected patient sera including aa 56-71, 154-170, and 193-204 using a deep-sequencing method [50]. Identification of antibodies against these regions of NS1, along with the highly conserved sequences among the four serotypes, gives precedent for the importance of these regions in protecting against DENV disease.

### Predicted Surface Exposure of Conserved Regions of DENV NS1 Protein

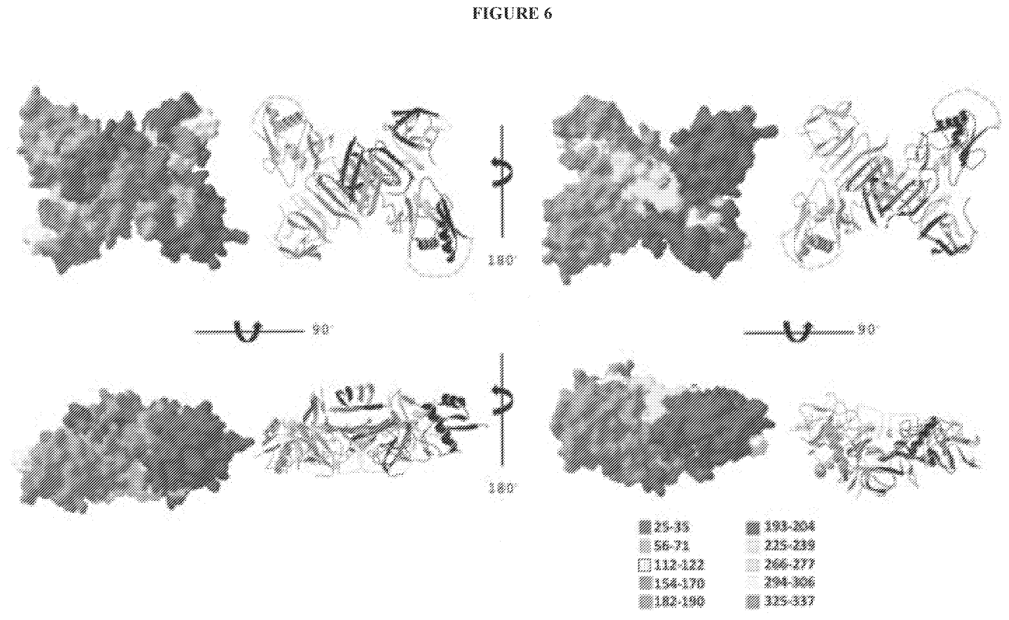

In order to investigate the potential for each of the conserved DENV NS1 regions to be surface exposed on the protein, the inventors mapped each peptide to the previously published structure of the DENV-2 dimer (PDB ID 406B) [51]. The location of each peptide is shown mapped to DENV-2 in FIG. 6 (individual peptides are in Supplemental FIG. S1). Peptides 25-35, 56-71, 112-122, 154-170, 225-239, 294-306, 325-337 are all at least partially surface exposed in the dimer structure, while peptides 182-190, 193-204, and 266-277 are mostly hidden within the structure.

Qβ VLPs Displaying NS1 Peptides are Immunogenic in Mice without Exogenous Adjuvant

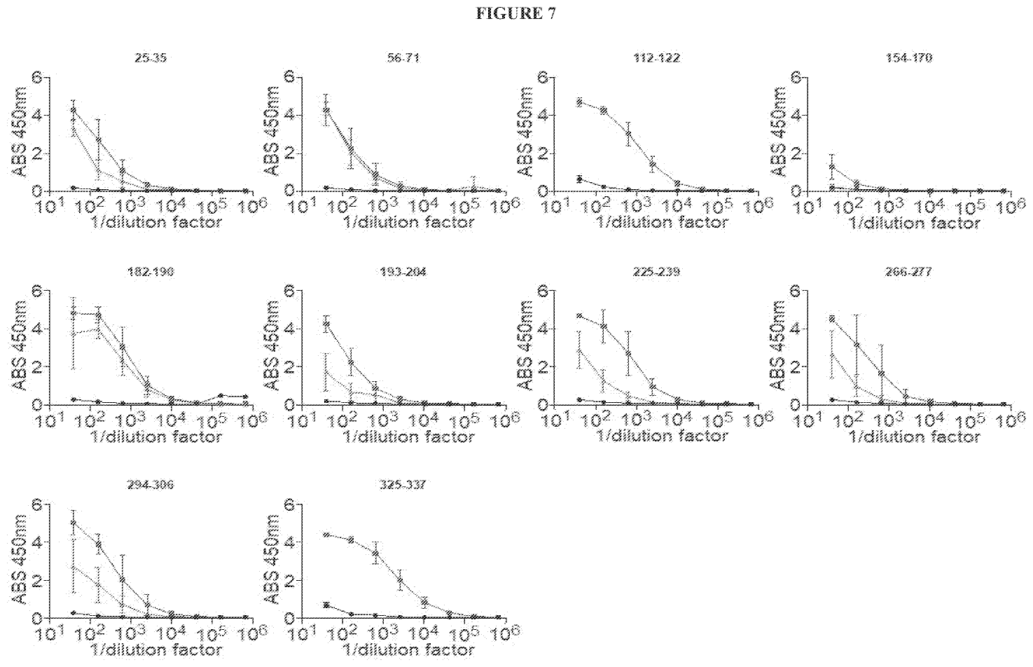

In order to investigate the potential of these conserved regions to elicit antibodies that could bind to DENV NS1, the inventors next generated synthetic peptides for each with a (Gly)₃ Cys linker on the C-terminus of the peptide in order to facilitate chemical conjugation to bacteriophage Qβ VLPs. Chemical conjugation to Qβ VLPs was carried out as described in the methods and successful conjugation was confirmed by SDS-PAGE and Coomassie staining (Supplemental FIG. S2). Mice were then immunized intramuscular with 5 μg/mouse without exogenous adjuvant two times at 3-week intervals, and blood was collected to assess antibody titers to cognate peptide (FIG. 7, red lines). For the remainder of this report, we will refer to these vaccines collectively as Qβ-NS1-PEPs. Three weeks after the first immunization (day 21), we observed IgG antibody responses to all Qβ-NS1-PEPs except Qβ-112-122 and Qβ-154-170 (FIG. 7, red lines). Three weeks after the second immunization (day 42), antibody titers of animals were tested again for antibody response to cognate peptides (FIG. 7, blue lines). After second immunization, all Qβ-NS1-PEPs showed high-titer antibody responses, except Qβ-154-170, which showed negligible antibodies against cognate peptide. Animals were then sacrificed, and sera were collected for further studies.

### Binding of Qβ-VLP-NS1-PEP Vaccine-Elicited Sera to Hexameric Recombinant NS1

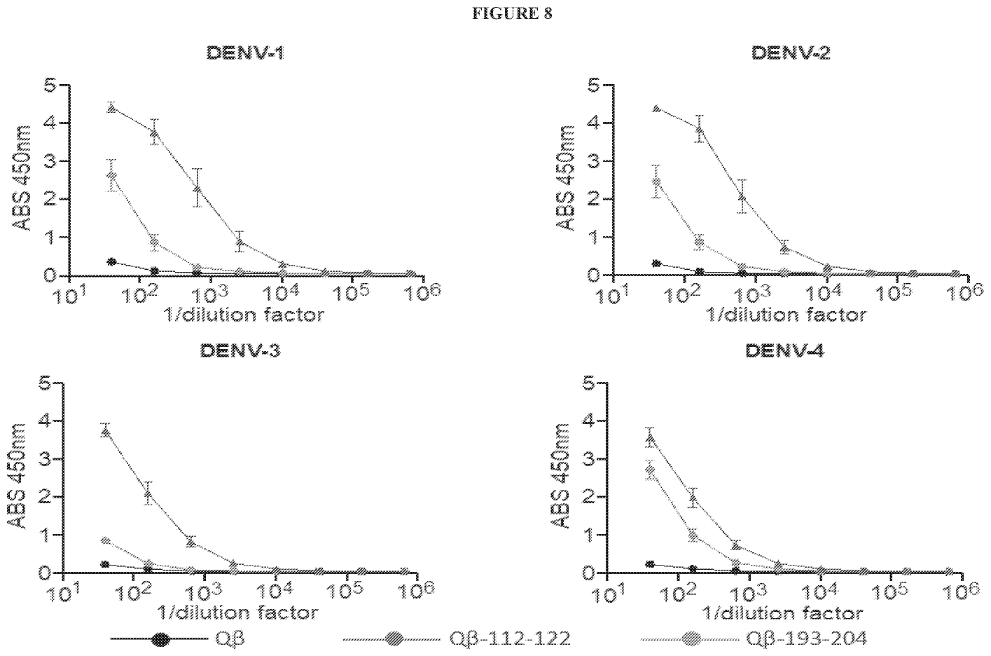

Having found that the Qβ-VLP-NS1-PEPs are immunogenic in mice and elicit antibodies that bind to their cognate peptide by ELISA, the inventors next investigated the ability of these immunogens to elicit antibodies that bind to DENV NS1 proteins. Using commercially available recombinant hexameric DENV NS1 proteins from all four DENV serotypes, the inventors examined mouse immune sera for binding by ELISA. A pool of sera (3 weeks post second immunization) was tested from each immunogen group to identify those immunogens that showed binding above Qβ VLP-immunized mouse sera (Supplemental FIG. S3). From this, the inventors identified sera from Qβ-112-122- and Qβ-193-204-immunized mice as capable of binding to DENV-NS1. To further investigate this, we performed ELISA with sera from the individual mice immunized with Qβ-112-122 and Qβ-193-204 (FIG. 8) against NS1 from all four DENV serotypes. Here, we found Qβ-112-122 to be highly immunogenic to all four serotypes of DENV NS1. Qβ-193-204, however, was less immunogenic than Qβ-112-122, but still bound to DENV NS1 from all four serotypes. Interestingly, Qβ-112-122 and Qβ-193-204 were least immunogenic against NS1 from DENV-3.

### Binding of Qβ-VLP-NS1-PEP Vaccine-Elicited Sera to DENV-Infected Cells

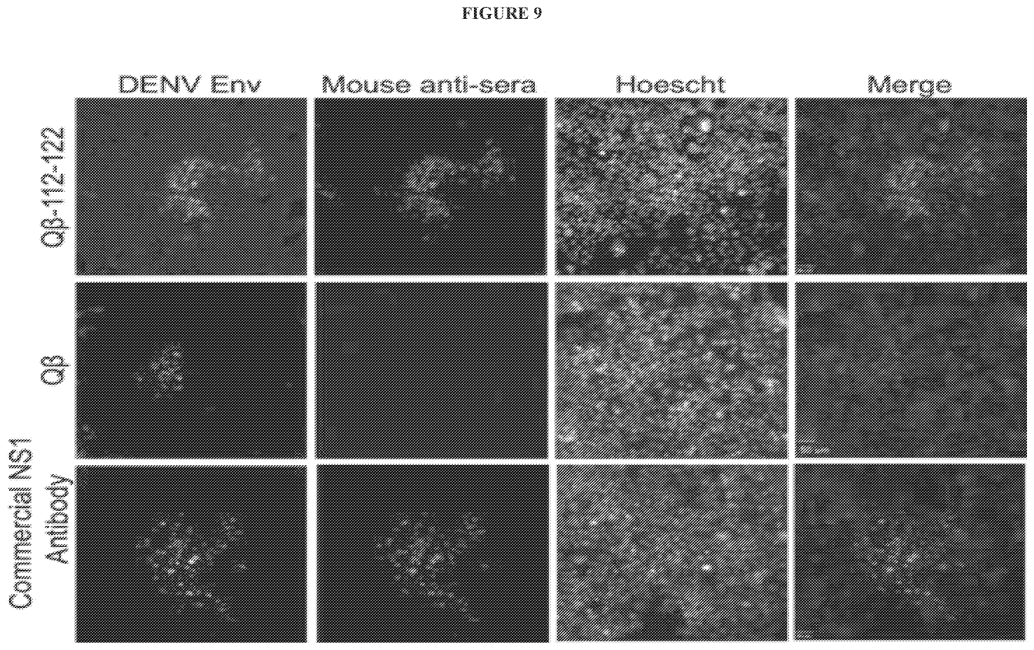

The tests thus far have compared the ability of the sera antibodies to bind soluble hexameric NS1 protein from all four serotypes of DENV. However, NS1 in a dimeric form exists on the surface of DENV-infected cells as well as intracellularly. Here, the inventors investigated whether or not antibodies from Qβ-VLP-NS1-PEPs bound to cell-associated NS1 in DENV-infected cells. Human embryonic kidney 293 (HEK 293) cells were infected with DENV from DENV-2 NGC at an MOI of 100 for 72 h. DENV-2 NGC was used since DENV-2 is the most commonly used serovar for laboratory studies. Infected cells were treated with a pan-flavivirus virus envelope antibody, 4G2, to identify infected cells. Additionally, sera from Qβ-VLP-NS1-PEP-immunized mice were added to the infected HEK 293 cells. Immunofluorescence microscopy was then performed to identify DENV-infected cells, as well as to identify whether our Qβ-VLP-NS1-PEP sera bound to the DENV-infected cells. Of our ten vaccine candidates, only sera from mice immunized against PEPs 112-122 bound to DENV-infected cells (FIG. 9 and Supplemental FIG. S4).

### DISCUSSION

DENV remains a severe threat to millions of people worldwide, yet a safe and effective vaccine that is useful for population-based vaccine campaigns has yet to be developed. A multitude of vaccine strategies targeting NS1 have been applied in order to solve this worldwide crisis, including protein, subunit peptide and DNA vaccines [12,17,18,21-28]. In addition to the current vaccine strategies, monoclonal and polyclonal sera have also been tested for efficacy of protection against severe DENV infections [12,17-20,52]. These methods have resulted in positive outcomes and survival of animals, indicating an important role for antibodies in the protection of DENV disease. However, while antibodies may aid in protection, antibodies that bind to the DENV envelope protein can also be detrimental and enhance infection through binding of host Fcγ receptors in a phenomenon coined Antibody-Dependent Enhancement (ADE) of infection. This supports the notion that antibody responses, while important, must be specific and safe to protect against DENV infection.

In earlier research, the inventors utilized deep sequence-coupled biopanning methods (DSCB) to identify regions of the DENV genome in which patients made antibodies against [37,50]. The envelope (E) protein, specifically the Fusion Loop on the envelope protein, was a particularly immunodominant region; a result that has been observed in other studies [53-57]. Other immunodominant regions that have been identified are the wing domain (WD) portion of NS1 (aa 112-122), and the tail region of NS1 (aa 325-337), in which the inventors identified a large number of patients making antibodies to these regions as well. These regions of the WD and NS1 tail are also highly conserved regions of the NS1 sequence, and pursuant to the present invention, the inventors investigated these along with other conserved regions.

The inventors hypothesized that targeting conserved regions of NS1 would be a strategy for eliciting antibodies that would recognize NS1 from all four DENV serotypes. However, only aa 112-122 peptide was able to elicit antibodies that bound to NS1 in both the soluble and infected cell-associated forms. These results suggest that the other conserved regions of NS1 are not antigenically available for antibody binding. The WD of NS1 was the most successful vaccine that was identified (aa 112-122) due to the high level of antibody response in immunized mice, as well as the sera's ability to bind soluble hexameric NS1 via ELISA and DENV-infected HEK293 cells. This was based off of previous research showing that the WD is a highly immunogenic epitope in all serotypes of DENV [39-48]. One obstacle of the WD is the amount of immunodominant antibodies and the uncertainty of the role it plays in DENV pathogenesis and whether these antibodies are indeed protective.

However, Lai et al. has shown that by modifying the WD region, protection from the severity of DENV disease in mice [18] in some capacity. This modified WD epitope is a region, along with other modifications of the WD, that should be tested with our bacteriophage VLP technology. Additionally, an asparagine has been identified as a glycosylation site essential in the endocytosis and pathogenic function of NS1 [58]. These sites and others may be appropriate for additional investigation as vaccine targets.

In previous research, the tail region of NS1 (aa 325-337) was highly selected by individuals who had been infected with their first DENV infection [50]. Additionally, antibodies from patients with secondary DENV infection had a binding profile similar to that of primary infected DENV patients, when sera were tested via ELISA [50]. However, the present studies show that the immunization against the NS1 tail region (aa 325-337) elicited high antibody titers to the cognate peptide, but these antibodies insufficiently bound to soluble NS1 and DENV-infected cells. This may be due to the fact that the Qβ VLP vaccines are displaying the peptide epitopes in a linear fashion. Additionally, the peptide ELISAs test the binding of sera antibodies to the linear peptides on the surface of the plate. However, in previous work the peptide ELISAs testing human sera against the NS1 tail peptide were testing sera antibodies in the same way [50]. This indicates that humans naturally infected with primary and secondary DENV infections made antibodies that were capable of binding to the linear form of the NS1 tail epitope. One explanation for this observation may be location of this NS1 tail region on the NS1 dimer. Based off of our structural analysis in FIG. 2, the surface-exposed regions of NS1 tail region (red) seems to be on the outer edges of the NS1 dimer. However, our studies here are also testing antibody binding to the hexameric form of NS1. These important binding sites may be hidden in the hexameric form of soluble NS1.

The benefit of the bacteriophage VLP platform of the present invention, compared to technologies such as immunization with whole protein, is the ability to make specific antibodies to selected epitopes. This specificity allows for control of the antibody profile such that it can be functional, yet safe by avoiding pathogenic antibodies during DENV infection. Additionally, previous work in our lab and others has shown that bacteriophage VLP-based vaccines induce high-titer, long-lasting antibody responses that do not require the addition of exogenous adjuvant, making our system relatively fast, easy, and safe to develop [31,32,59,60]. The present results show that our VLP-based NS1 vaccines have the capacity to produce high-titer antibodies against specific DENV NS1 epitopes and these antibodies bind to both hexameric soluble NS1 and cell-associated NS1. Further analysis of the functional characteristics and abilities of these vaccine candidates will be assessed in future studies. Functional assays such as protection against endothelial barrier disruption, peripheral blood mononuclear cell activation, and the protective capacity of these vaccines in an animal model of NS1-mediated pathogenesis are being investigated. Furthermore, other epitopes of the DENV NS1 protein as disclosed may identify other regions that are particularly protective in severe DENV disease.

### Experiments Utilizing Non-Conserved DENV NS1 Immunogenic Peptides

In these experiments, the inventors conducted an unbiased screen of overlapping peptides. They made a collection of VLPs for these peptides and then performed small scale pilot immunization experiments to determine if the VLPs elicited antibodies that bound to NS1. VLPs that elicited NS1-binding antibodies were then used to immunize more animals for further experiments. Sera from these down-selected VLPs were then used to assess binding to DENV-infected cells by immunofluorescence microscopy.

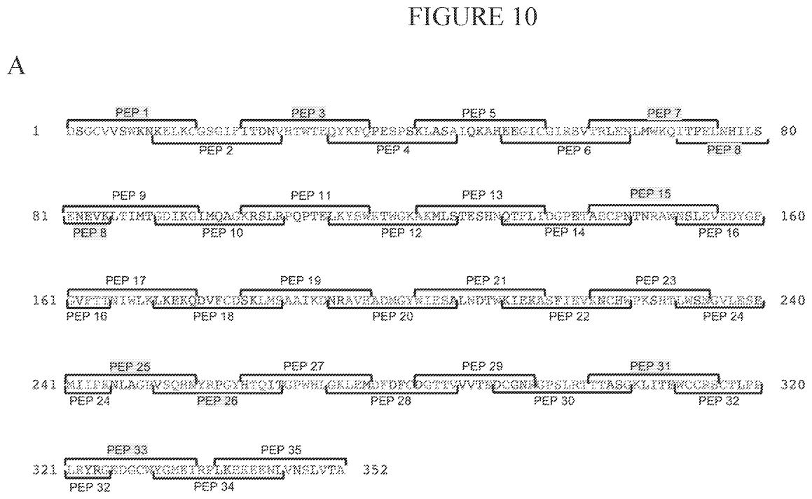

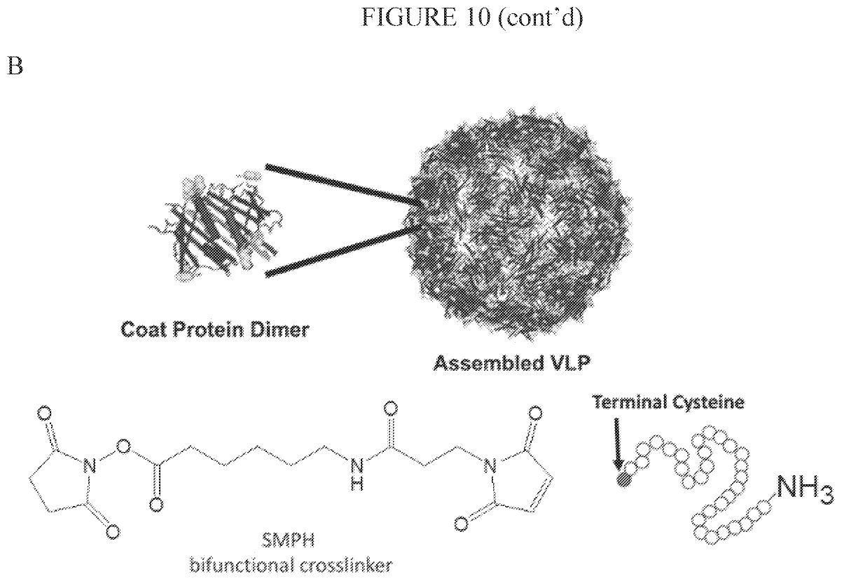

The inventors hypothesized that there may be peptides from Dengue virus NS1 that would be good vaccine candidates but that could not be deduced rationally, but could only be known if empirically. So, the inventors did the unbiased screening described below. They synthesized 35 overlapping peptides that spanned the entire NS1 protein (this is the unbiased aspect of this). These are presented in FIG. 10A. These peptides were then used to make 35 individual vaccines (peptides were conjugated to Qbeta VLPs). These were conjugated to the Qbeta VLP using methods otherwise described herein, with the majority of the peptide successfully conjugating to VLP. The inventors prioritized several of these VLPs to move into further testing based on the observation that these immunogenic peptides were able to elicit antibodies that bound to recombinant NS1 protein, indicating that these conjugated VLPs were worth testing further to see if they could protect mice. These are tested to determine efficacy in blocking NS1 activity or protecting against dengue virus pathology in an animal.

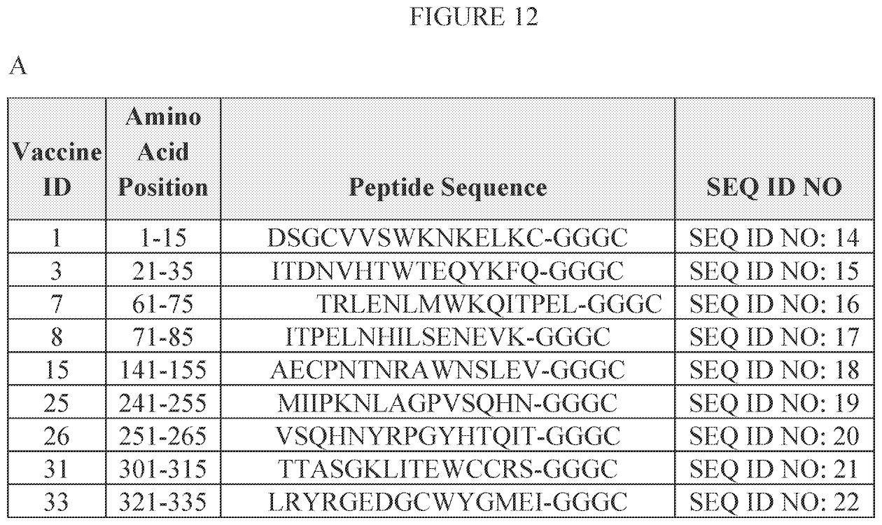

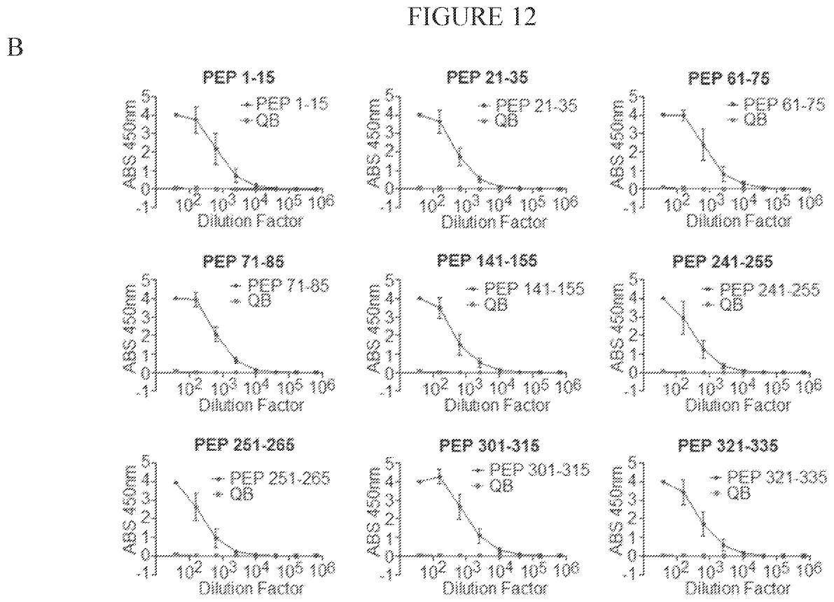

Of the 35 peptides spanning the entirety of NS1 (FIG. 10A), the inventors identified 9 that were thought to be especially worth pursuing further, because they bound well to recombinant NS1 protein. These 35 peptides are shown in FIG. 12B as pep 1-35. Although 9 peptides so far have been prioritized for further testing, any of the 35 peptides that elicited antibodies that bound to NS1 are theoretically good candidates.

### 2. Materials and Methods

### Synthesis of NS1 Peptides

The DENV-2 NS1 protein (Accession #: NP_739584.2) was used to identify overlapping 15 amino acid peptides for investigation. A C-terminal (Gly)4-Cys linker was synthesized on each peptide of interest to allow for the chemical conjugation to the surface of Qβ VLPs.

### 2.2 Qβ VLP Vaccine Production

Qβ VLPs were produced as previously described (Frietze et al., Pub Med. ID PMID: 31625095). Briefly, Escherichia coli (E. coli) was transfected with a plasmid that expressed Qbeta VLP. Bacteria cultures were grown until an optical density (OD) of 600 was reached in which cultures were then induced with 0.5 mM Isopropyl-β-D-thiogalactoside (IPTG). Cultures were then pelleted and sonicated in the presence of 10% deoxycholate (DOC). Sonicated solution was then incubated at 37° C. for 1 hour in a vinal concentration of 2 mM magnesium chloride (MgCl2) and 10 mg/mL of DNase. Samples were spun and supernatants were added to ammonium sulfate to 60% saturation overnight at 4° C. Ammonium sulfate and debris was removed through ultracentrifugation and samples were frozen at −80° C. until size exclusion chromatography was performed. Qβ samples were purified through a sepharose bead column (need to look up size/number) and fractionated. Fractions containing VLPs were combined with ammonium sulfate overnight to reach 70% saturation. Samples were then ultracentrifuged to remove ammonium sulfate. Buffer exchange was performed with PBS and snakeskin dialysis tubing (ThermoFisher) to remove remaining ammonium sulfate. Qβ stocks were then LPS depleted and concentration was determined by SDS-PAGE gel. Stocks were stored at −20° C. until used.

Peptides of interest were synthesized by Genescript and contain a C-terminal tri-glycine linker at the C-terminal end of the peptide. Peptides were then conjugated to the surface of Qβ VLPs through the use of succinimidyl-6-((b-maleimidopropionamido)hexanoate (SMPH). SMPH is a bifunctional crosslinker that binds the surface exposed lysines on Qβ VLPs and bind the C-terminal cysteine containing a fee sulfhydryl group (FIG. 10B. Qβ VLPs were conjugated with SMPH for two hours with rocking at room temperature and access SMPH was removed by centrifugation using Amicon Ultracel 100K filtration devices (Merck). Peptides were then added to VLPs overnight at 4° C. overnight. Excess peptide was removed through centrifugation with Amicon Ultracel 100k filtration devices. Conjugation efficiency and concentration of vaccines were determined through SDS-PAGE electrophoresis.

### Enzyme-Linked Immunosorbent Assays (ELISAs)

Peptide ELISAs were completed as previously stated (Warner et al PMID: 33008118). Briefly, 96-well ELISA plates were coated with 0.5 μg/50 μL of Streptavidin (Invitrogen Cat #434302) in phosphate buffered saline (PBS) overnight at 4° C. Wells were washed three times with PBS and 1 ug/50 μL of succinimidyl-6-((b-maleimidopropionamido)hexanoate (SMPH; Milipore Sigma) in PBS was added to each well for 1 hour at room temperature with rocking. Plates were washed three times with PBS and cognate peptide was diluted in PBS and added to each well at a concentration of 1 ug/50 μL for two hours at room temperature with rocking. Plates were then washed three time with PBS, and blocked with 150 μL of 0.5% Milk/PBS overnight in 4° C. Sera samples were then diluted in 0.5% Milk/PBS starting with 1:40 dilution, followed by 4-fold dilutions up to 1:655,360. Plates were washed twice with PBS and sera was plated at 50 μL volumes for two hours at room temperature with shaking. Plates were washed five times with PBS and 50 μL of secondary Goat anti-mouse antibody conjugated with horseradish peroxidase (HRP; Jackson ImmunoResearch) was added at a 1:5000 dilution in 0.5% Milk/PBS for 45 minutes at room temperature with rocking. Wells were washed 5 times with PBS and 50 μL of soluble TMB was added to each well for 15 minutes with rocking. Enzymatic reaction was quenched using 50 μL of 1% Hydrochloric acid solution. Plates were analyzed at 450 nm wavelength with an accuSkan plate reader (ThermoFisher).

For ELISAs testing the binding of antibodies to DENV NS1, soluble NS1 proteins were produced in HEK 293 cells and purchased from Native Antigen Company. ELISA plates were coated with 0.16 μg/well of NS1 in PBS and incubated overnight at 4° C. Plates were washed 3 times with PBS and wells were blocked with 100 μL of 0.5% Milk/PBS for one hour at room temperature with rocking. The remaining steps of the ELISA followed the protocol as above.

### Animals

Immunizations for Sera Antibodies: 6-8 week old male and female BALB/c mice (n=3 per group) were immunized intramuscularly in the hind leg twice, at three week intervals with 5 μg of vaccine in 50 μL volumes. At days 21 blood samples were taken via retro-orbital bleeds to analyze sera antibodies via peptide ELISA. Day 42, animals were sacrificed via cardiac puncture to collect sera.

### Cells and Viral Stocks

Human embryonic kidney (HEK-293) cells were purchased through American Type Culture Collection (ATCC, CRL-1573). Cells were kept in complete media containing Eagle's Minimum Essential Medium (MEM, ATCC) supplemented with 10% fetal bovine serum (FBS). Aedes albopictus C6/36 cells were purchased through ATCC (CRL-1660) and grown in complete media containing MEM supplemented with 0.1% gentamycin reagent solution (Gibco), 1% of 100× MEM Non-essential amino acids (Gibco), 1% of 100× 200 mM L-glutamine (Gibco) and 10% FBS.

Viral stocks of DENV type 2 (NGC Proto), DENV-3 (Sleman), and DENV-4 (Thailand) were kindly provided by Dr. Kathryn Hanley at New Mexico State University (NMSU). DENV-2 strain 16681 was received from University of Texas Medical Branch repository. Viral stocks were propagated in C6/36 cells and stored at −80° C. in C6/36 media supplemented with 1× sucrose phosphate glutamate (SPG) buffer. Viral stocks were verified through sequencing rt-PCR product using primers.

### Anti-NS1 Antibodies Binding to DENV-Infected HEK-293 Cells

HEK-293 cells were plated at 25,000 cells/well on Cellvis 96-well glass bottom chimney plates overnight. Cells were then washed once with HEK media and infected with 20 μL of DENV-2 NGC Proto at an MOI of 100. Plates were rocked every 5 minutes for a total of 20 minutes. After incubation, 150 μL of HEK media was added to each well and plates were incubated for three days at 37° C. HEK media was removed and cells were washed once with PBS. Cells were then fixed with 200 μL of 100% methanol per well for 20 minutes, two times. Fixed cells were washed once with PBS, followed by blocking with 200 μL of 2% goat sera for one hour. Goat sera was removed and cells were washed three times with PBS. Sera from immunized mice was added at 1:40, 1:80, 1:160 and diluted in 2% goat sera. Commercial antibodies were added at the following dilutions: Mouse anti-Flavivirus NS1 antibody 1:500 (Abcam, ab214337), rabbit anti-flavivirus envelope protein antibody 1:1000 (4G2; The Native Antigen Company). Plates were washed three times with PBS and secondary antibodies were diluted at 1:200 in 2% goat sera for 45 minutes. Secondary antibodies used were Alexa Fluor 647-conjugated goat anti-rabbit IgG (Jackson ImmunoResearch) and goat anti-mouse IgG conjugated with Alexa Fluor 488 (Abcam). Wells were washed three times with PBS and 50 μL of 1:2,000 dilution of Hoechst (Invitrogen) was added to each well for 10 minutes. Plates were washed three times with PBS and 150 μL of PBS was added to each well for imaging. Plates were analyzed using standard methods.

### Results

### Strategy for Unbiased Identification of NS1 Peptide Epitopes of Interest

DENV NS1 protein is a 352 amino acid protein is detectable in DENV infected cells as well as secreted as a hexameric protein. Because this sequence of NS1 is variable among the DENV serotypes, we decided to focus our efforts to begin on DENV-2. We chose DENV-2 because many of the animal models available for DENV use a DENV-2 strain fro infection. We chose to synthesize 15 amino acid peptides, overlapping by 5 amino acids, providing us full coverage of the DENV-2 NS1 protein with 35 peptides (FIG. 10A). These peptides were commercially synthesized with a (Gly)₄-Cys linker on the C-terminus to allow for chemical conjugation using the free sulfhydryl group provided by the Cys residue. Each peptide was conjugated to pre-formed Qβ VLPs, resulting in 35 individual immunogens for further testing (FIG. 10B). Conjugation was confirmed by SDS-PAGE and Coomassie staining, demonstrating a shift in electrophoretic mobility indicating conjugation of 1-3 peptides per individual coat protein.

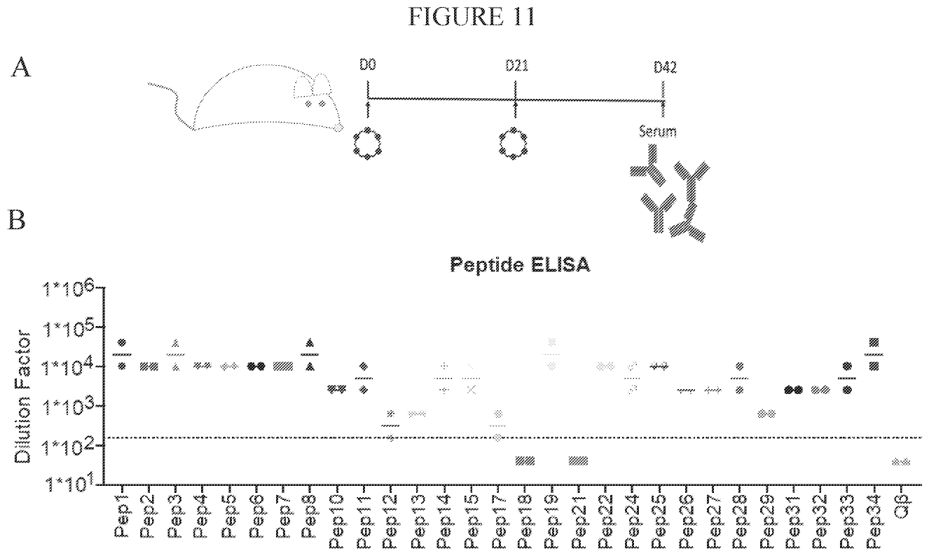

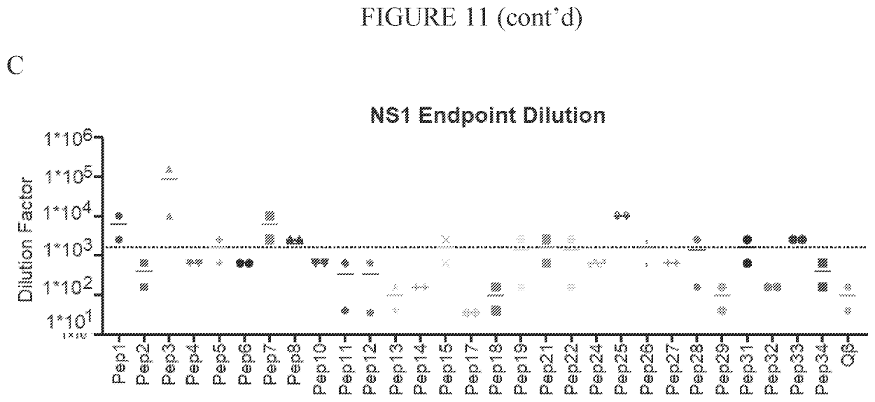

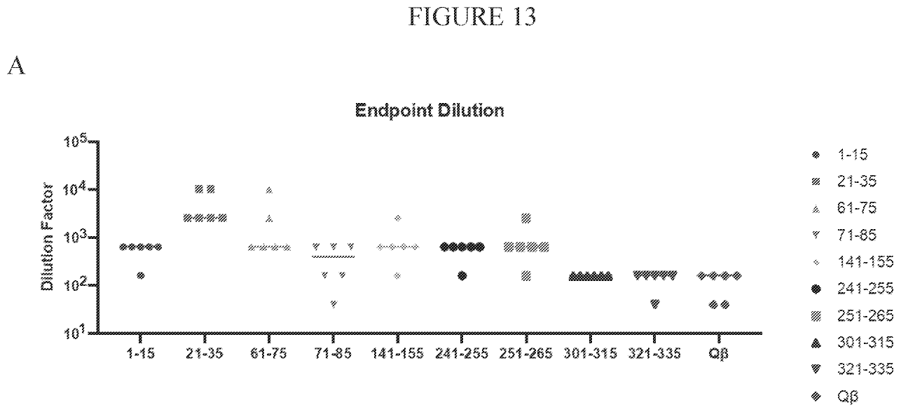

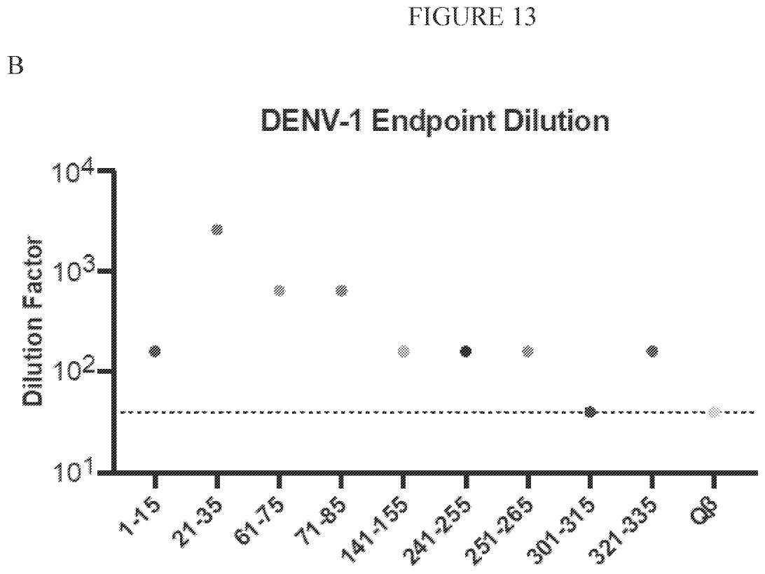

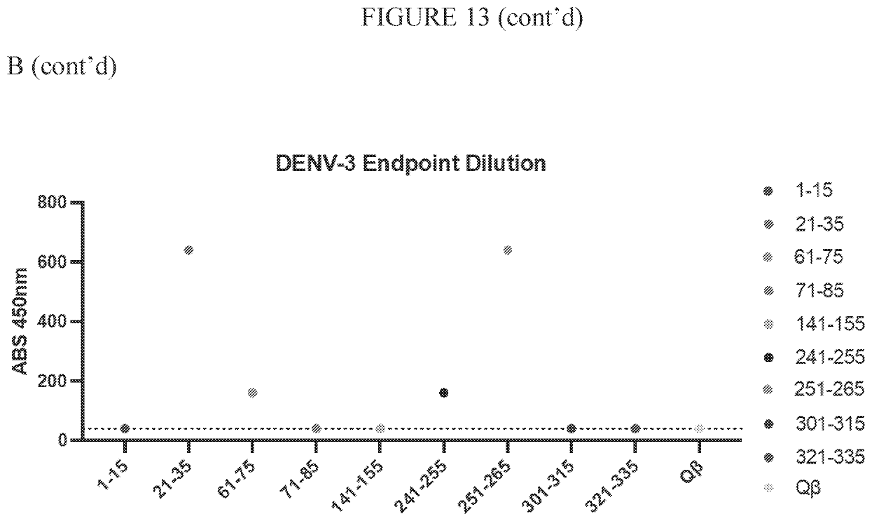

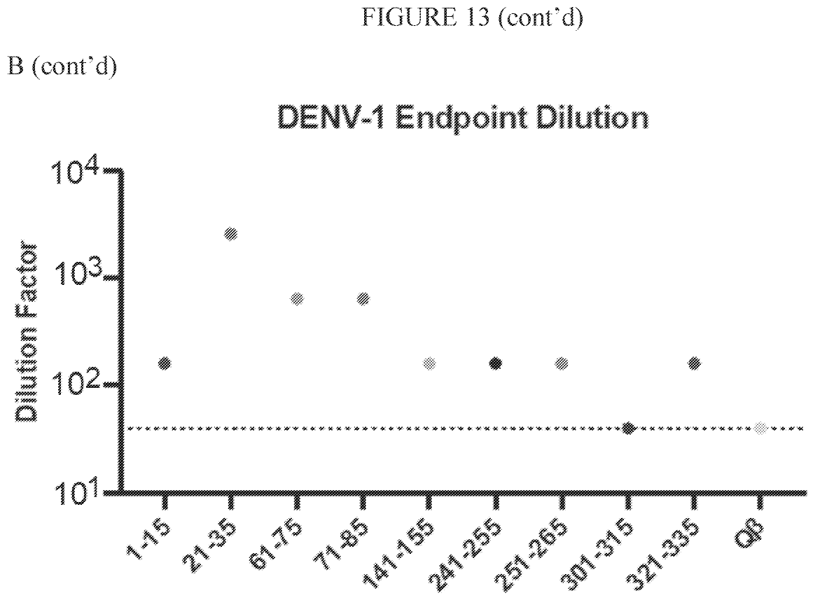

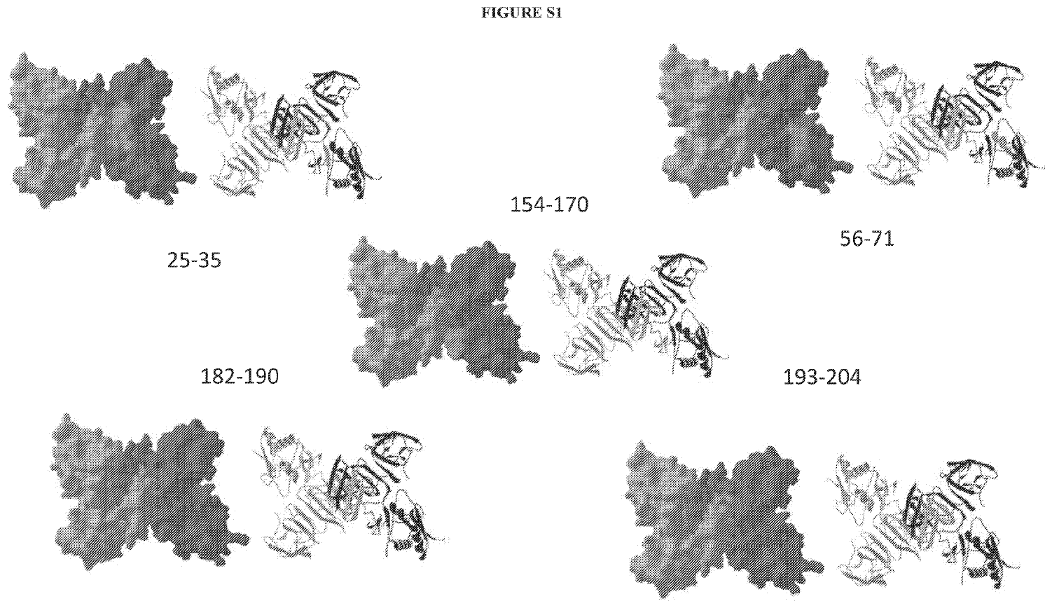

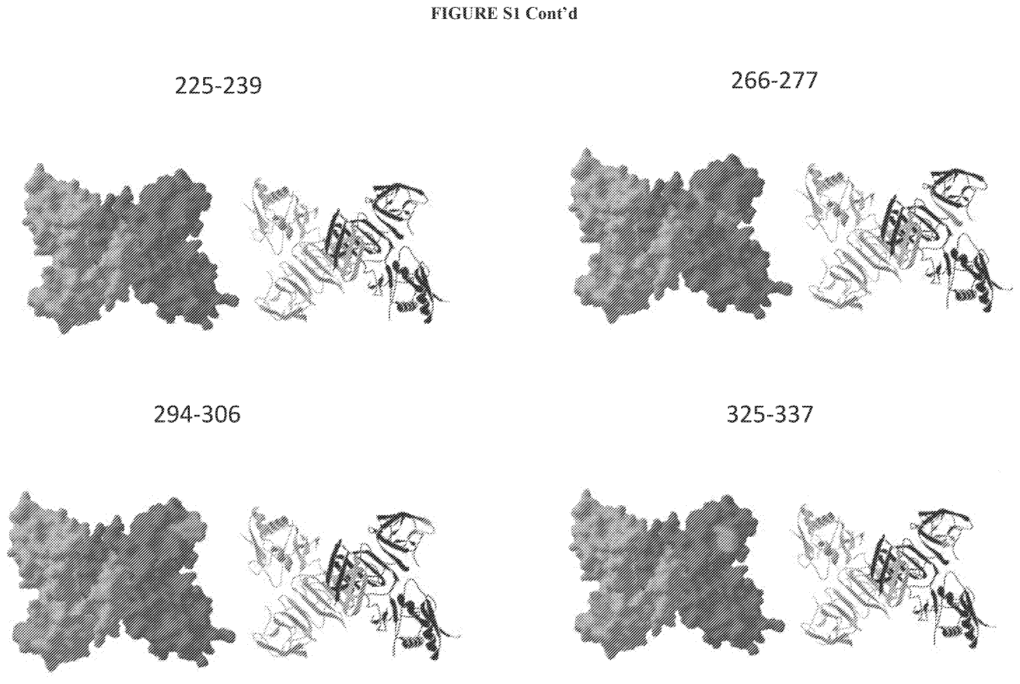

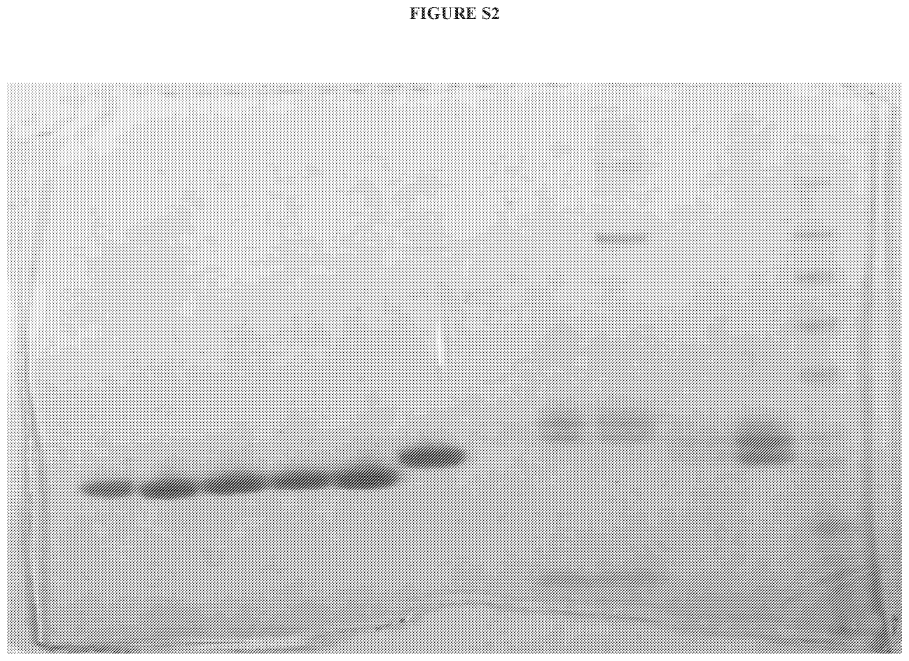

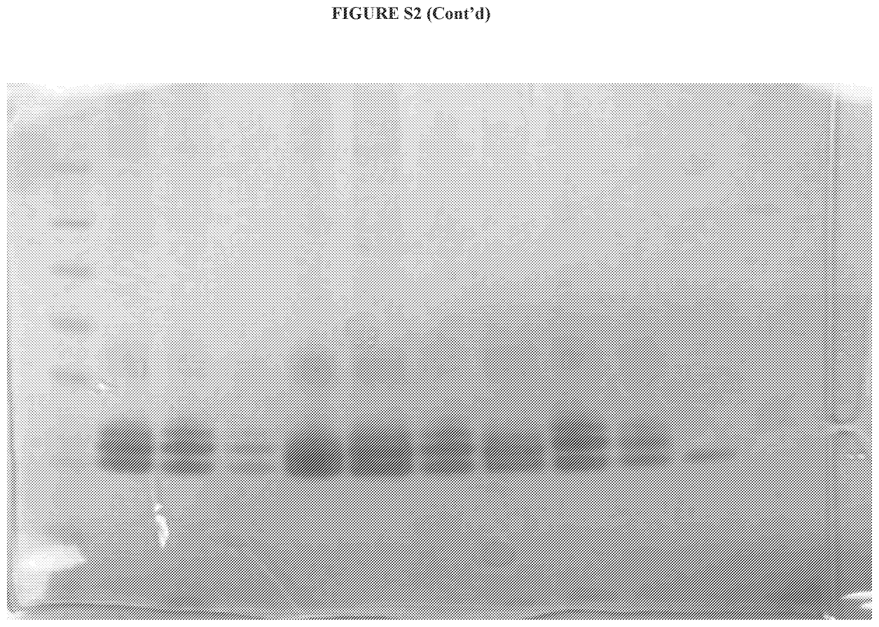

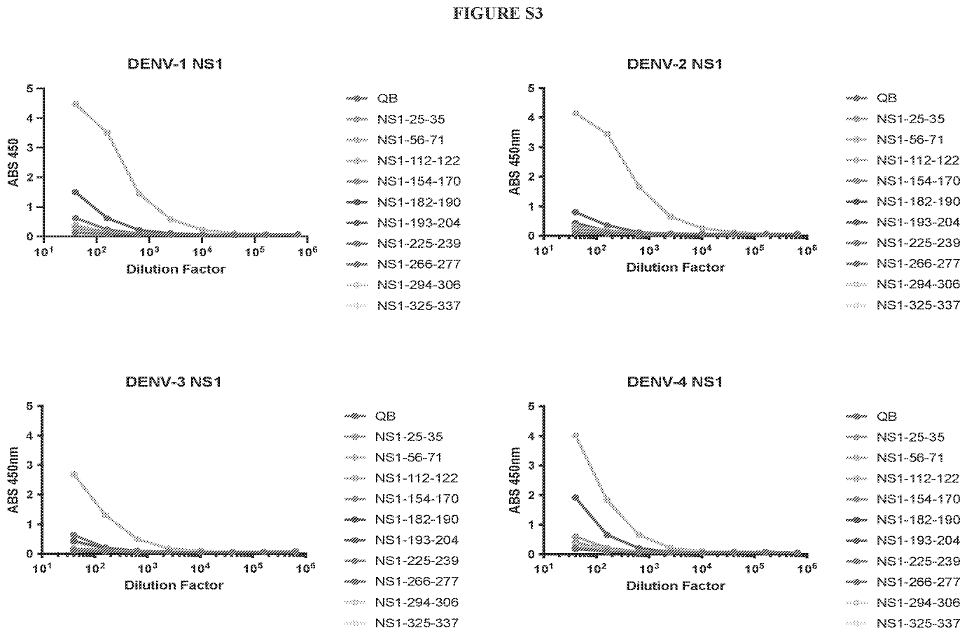

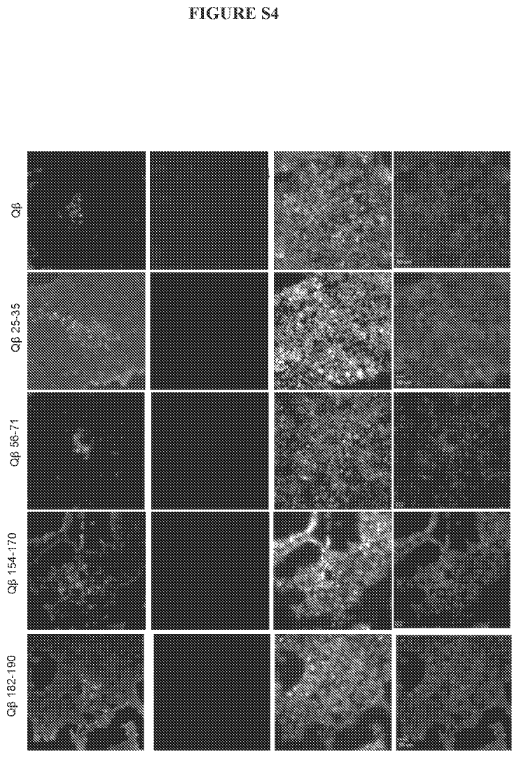

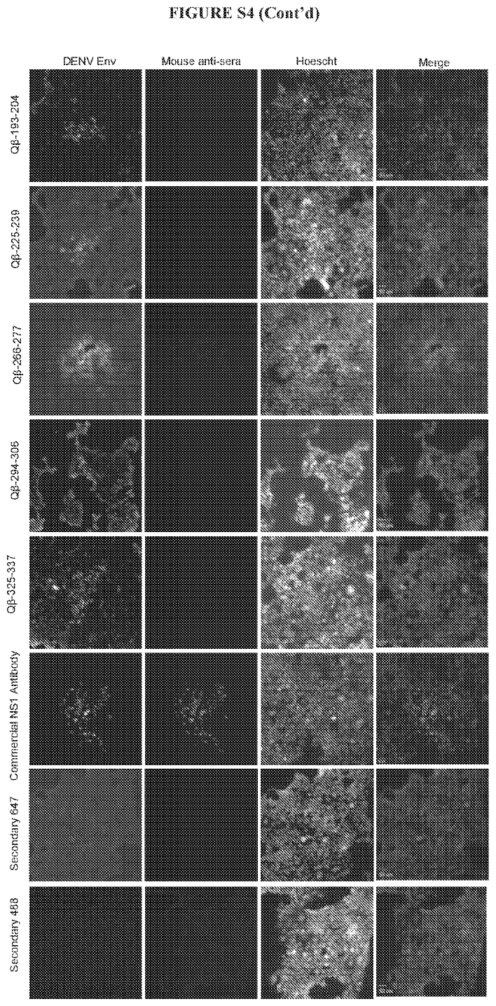

The inventors then screened the DENV peptide library created. 6-8 week old female BALB/c mice (n=2/group) were immunized twice with 5 μg/50 μL of Qβ VLP vaccines on day 0 and day 21 FIG. S(A). 42 days post immunization, mice were sacrificed and sera was collected for antibodies (A). ELISAs against cognate peptide were performed; dotted line represents 1:160 dilution of sera and indicates the cut off for acceptable antibody titers FIG. 11 (B). PEP library was screened for binding to soluble, hexameric DENV-2 NS1. Dotted line represents the cut off for acceptable titers at 1:1600 FIG. 11 (B). Immunization of BALB/c mice with top 9 Qβ VLP vaccines (conjugating immunogenic peptides of SEQ ID NO: 14-22) induced high titer antibodies against cognate peptide. The top nine Qβ-PEP vaccines were identified from the initial screening and highlighted on the dimeric structure of DENV-2 NS1 FIG. 12(A). The top nine Qβ-PEP vaccine amino acid positions and peptide sequences are displayed in the table of FIG. 12 (B). 6-8 week old BALB/c mice were immunized with Qβ-PEP vaccines from the top nine binders n=6 mice/vaccine (3 male, 3 female). Peptide ELISA was used to test binding of against cognate peptide comparing vaccine-sera (blue) and control sera of mice immunized with Qβ only (red) FIG. 12 (C). The top 9 VLP vaccines (which conjugate immunogenic peptides SEQ ID Nos. 14-22) against DENV NS1 bind DENV-infected cells and hexameric forms of DENV NS1. Human embryonic kidney (HEK) 293 cells were infected with DENV-2 NGC Proto at an MOI of 100. Infected cells were then fixed and stained with anti-DENV NS1 sera from vaccinated mice (green) or positive control monoclonal anti-flavivirus NS1 antibody (green), and rabbit anti-flavivirus envelope protein antibody (4G2) for detection of viral infection (red) FIG. 13 (A). Binding of top 9 VLP vaccines to soluble hexameric NS1 from DENV-2 FIG. 13 (C). Pooled sera samples of top 9 vaccines against soluble, hexameric NS1 from DENV-1, DENV-3, and DENV-4 FIG. 13 (D).

### Immunogenicity of Q)/VLPs Displaying Unbiased NS1 Peptide Epitopes

Having successfully engineered 35 Qβ VLPs displaying NS1 peptides, the inventors next performed an initial immunogenicity screen of the immunogens in mice. Their previous experience immunizing mice with Qβ VLPs has shown very little to no within-group variation in antibody titer, so we were able to reduce the number of mice per group to 2. Mice were immunized twice, 3 weeks apart, and serum was collected 3 weeks after boost. All but 2 of the immunogens tested elicited antibodies that bound to cognate peptide above Qβ negative control. Next, the inventors assessed binding of immune sera to recombinant hexameric NS1 from DENV-2 by ELISA. In order to prioritize immunogens for further investigation, the inventors established the following criteria: (1) both mice in a group showing endpoint titer higher than 1:160 to cognate peptide, and (2) detectable binding to recombinant NS1 above background. Peptides chosen for further investigation were used to immunize additional mice to obtain sufficient sera to perform additional tests, So far, binding to DENV-2 infected cells was assessed by immunofluorescence microscopy, showing binding above background indicating these peptides elicit antibodies that both bind to soluble hexameric DENV NS1 protein as also cell-associated DENV NS1 protein in infected cells.

Future experiments will assess the protective capacity of these VLPs in animal models of DENV infection.

### REFERENCES

### First Set of References

1. Glasner D R, Puerta-Guardo H, Beatty P R, Harris E. The Good, the Bad, and the Shocking: The Multiple Roles of Dengue Virus Nonstructural Protein 1 in Protection and Pathogenesis. Annu Rev Virol. 2018; 5(1):227-53. Epub 2018/07/25. doi: 10.1146/annurev-virology-101416-041848. PubMed PMID: 30044715.
2. Chen H R, Lai Y C, Yeh T M. Dengue virus non-structural protein 1: a pathogenic factor, therapeutic target, and vaccine candidate. J Biomed Sci. 2018; 25(1):58. Epub 2018/07/24. doi: 10.1186/s12929-018-0462-0. PubMed PMID: 30037331; PMCID: PMC6057007.
3. Libraty D H, Young P R, Pickering D, Endy T P, Kalayanarooj S, Green S, Vaughn D W, Nisalak A, Ennis F A, Rothman A L. High circulating levels of the dengue virus nonstructural protein NS1 early in dengue illness correlate with the development of dengue hemorrhagic fever. J Infect Dis. 2002; 186(8):1165-8. Epub 2002/09/16. doi: 10.1086/343813. PubMed PMID: 12355369.
4. Glasner D R, Ratnasiri K, Puerta-Guardo H, Espinosa D A, Beatty P R, Harris E. Dengue virus NS1 cytokine-independent vascular leak is dependent on endothelial glycocalyx components. PLoS Pathog. 2017; 13(11):e1006673. Epub 2017/11/09. doi: 10.1371/journal.ppat.1006673. PubMed PMID: 29121099; PMCID: PMC5679539.
5. Chen H R, Chao C H, Liu C C, Ho T S, Tsai H P, Perng G C, Lin Y S, Wang J R, Yeh T M. Macrophage migration inhibitory factor is critical for dengue NS1-induced endothelial glycocalyx degradation and hyperpermeability. PLoS Pathog. 2018; 14(4):e1007033. Epub 2018/04/27. doi: 10.1371/journal.ppat.1007033. PubMed PMID: 29702687; PMCID: PMC6044858.
6. Modhiran N, Watterson D, Muller D A, Panetta A K, Sester D P, Liu L, Hume D A, Stacey K J, Young P R. Dengue virus NS1 protein activates cells via Toll-like receptor 4 and disrupts endothelial cell monolayer integrity. Sci Transl Med. 2015; 7(304):304ra142. doi: 10.1126/scitranslmed.aaa3863. PubMed PMID: 26355031.
7. Modhiran N, Watterson D, Blumenthal A, Baxter A G, Young P R, Stacey K J. Dengue virus NS1 protein activates immune cells via TLR4 but not TLR2 or TLR6. Immunol Cell Biol. 2017; 95(5):491-5. Epub 2017/02/21. doi: 10.1038/icb.2017.5. PubMed PMID: 28220810.
8. Chen J, Ng M M, Chu J J. Activation of TLR2 and TLR6 by Dengue NS1 Protein and Its Implications in the Immunopathogenesis of Dengue Virus Infection. PLoS Pathog. 2015; 11(7):e1005053. Epub 2015/07/30. doi: 10.1371/joumal.ppat.1005053. PubMed PMID: 26226614; PMCID: PMC4520596.
9. Schlesinger J J, Brandriss M W, Walsh E E. Protection of mice against dengue 2 virus encephalitis by immunization with the dengue 2 virus non-structural glycoprotein NS1. J Gen Virol. 1987; 68 (Pt 3):853-7. doi: 10.1099/0022-1317-68-3-853. PubMed PMID: 3819700.
10. Henchal E A, Henchal L S, Schlesinger J J. Synergistic interactions of anti-NS1 monoclonal antibodies protect passively immunized mice from lethal challenge with dengue 2 virus. J Gen Virol. 1988; 69 (Pt 8):2101-7. doi: 10.1099/0022-1317-69-8-2101. PubMed PMID: 3404125.
11. Beatty P R, Puerta-Guardo H, Killingbeck S S, Glasner D R, Hopkins K, Harris E. Dengue virus NS1 triggers endothelial permeability and vascular leak that is prevented by NS1 vaccination. Sci Transl Med. 2015; 7(304):304ra141. doi: 10.1126/scitranslmed.aaa3787. PubMed PMID: 26355030.
12. Lai Y C, Chuang Y C, Liu C C, Ho T S, Lin Y S, Anderson R, Yeh T M. Antibodies Against Modified NS1 Wing Domain Peptide Protect Against Dengue Virus Infection. Sci Rep. 2017; 7(1):6975. Epub 2017/08/01. doi: 10.1038/s41598-017-07308-3. PubMed PMID: 28765561; PMCID: PMC5539099.
13. Falconar A K. The dengue virus nonstructural-1 protein (NS1) generates antibodies to common epitopes on human blood clotting, integrin/adhesin proteins and binds to human endothelial cells: potential implications in haemorrhagic fever pathogenesis. Arch Virol. 1997; 142(5):897-916. PubMed PMID: 9191856.
14. Falconar A K. Antibody responses are generated to immunodominant ELK/KLE-type motifs on the nonstructural-1 glycoprotein during live dengue virus infections in mice and humans: implications for diagnosis, pathogenesis, and vaccine design. Clin Vaccine Immunol. 2007; 14(5):493-504. doi: 10.1128/CVI.00371-06. PubMed PMID: 17329445; PMCID: PMC1865631.
15. Lin C F, Wan S W, Chen M C, Lin S C, Cheng C C, Chiu S C, Hsiao Y L, Lei H Y, Liu H S, Yeh T M, Lin Y S. Liver injury caused by antibodies against dengue virus nonstructural protein 1 in a murine model. Lab Invest. 2008; 88(10):1079-89. Epub 2008/08/04. doi: 10.1038/labinvest.2008.70. PubMed PMID: 18679379.
16. Lin Y S, Yeh T M, Lin C F, Wan S W, Chuang Y C, Hsu T K, Liu H S, Liu C C, Anderson R, Lei H Y. Molecular mimicry between virus and host and its implications for dengue disease pathogenesis. Exp Biol Med (Maywood). 2011; 236(5):515-23. Epub 2011/04/18. doi: 10.1258/ebm.2011.010339. PubMed PMID: 21502191.
17. Chuang Y C, Lin Y S, Liu H S, Wang J R, Yeh T M. Antibodies against thrombin in dengue patients contain both anti-thrombotic and pro-fibrinolytic activities. Thromb Haemost. 2013; 110(2):358-65. Epub 2013/06/06. doi: 10.1160/TH13-02-0149. PubMed PMID: 23740201.
18. Chuang Y C, Wang S Y, Lin Y S, Chen H R, Yeh T M. Re-evaluation of the pathogenic roles of nonstructural protein 1 and its antibodies during dengue virus infection. J Biomed Sci. 2013; 20:42. Epub 2013/06/27. doi: 10.1186/1423-0127-20-42. PubMed PMID: 23806052; PMCID: PMC3704815.
19. Frietze K M, Peabody D S, Chackerian B. Engineering virus-like particles as vaccine platforms. Curr Opin Virol. 2016; 18:44-9. doi: 10.1016/j.coviro.2016.03.001. PubMed PMID: 27039982.
20. Halstead S B. Safety issues from a Phase 3 clinical trial of a live-attenuated chimeric yellow fever tetravalent dengue vaccine. Hum Vaccin Immunother. 2018:1-5. Epub 2018/02/26. doi: 10.1080/21645515.2018.1445448. PubMed PMID: 29482433.
21. Wahala W M, Silva A M. The human antibody response to dengue virus infection. Viruses. 2011; 3(12):2374-95. doi: 10.3390/v3122374. PubMed PMID: 22355444; PMCID: PMC3280510.
22. Guzman M G, Alvarez M, Halstead S B. Secondary infection as a risk factor for dengue hemorrhagic fever/dengue shock syndrome: an historical perspective and role of antibody-dependent enhancement of infection. Arch Virol. 2013; 158(7):1445-59. Epub 2013/03/08. doi: 10.1007/s00705-013-1645-3. PubMed PMID: 23471635.
23. Halstead S B. Dengue Antibody-Dependent Enhancement: Knowns and Unknowns. Microbiol Spectr. 2014; 2(6). doi: 10.1128/microbiolspec.AID-0022-2014. PubMed PMID: 26104444.
24. Malavige G N, Ogg G S. Pathogenesis of vascular leak in dengue virus infection. Immunology. 2017; 151(3):261-9. Epub 2017/05/24. doi: 10.1111/imm.12748. PubMed PMID: 28437586; PMCID: PMC5461104.
25. Liu I J, Chiu C Y, Chen Y C, Wu H C. Molecular mimicry of human endothelial cell antigen by autoantibodies to nonstructural protein 1 of dengue virus. J Biol Chem. 2011; 286(11):9726-36. doi: 10.1074/jbc.M110.170993. PubMed PMID: 21233208; PMCID: PMC3058979.
26. Cheng H J, Lin C F, Lei H Y, Liu H S, Yeh T M, Luo Y H, Lin Y S. Proteomic analysis of endothelial cell autoantigens recognized by anti-dengue virus nonstructural protein 1 antibodies. Exp Biol Med (Maywood). 2009; 234(1):63-73. Epub 2008/11/07. doi: 10.3181/0805-RM-147. PubMed PMID: 18997103.
27. Akey D L, Brown W C, Dutta S, Konwerski J, Jose J, Jurkiw T J, DelProposto J, Ogata C M, Skiniotis G, Kuhn R J, Smith J L. Flavivirus NS1 structures reveal surfaces for associations with membranes and the immune system. Science. 2014; 343(6173):881-5. doi: 10.1126/science.1247749. PubMed PMID: 24505133; PMCID: PMC4263348.
28. Hertz T, Beatty P R, MacMillen Z, Killingbeck S S, Wang C, Harris E. Antibody Epitopes Identified in Critical Regions of Dengue Virus Nonstructural 1 Protein in Mouse Vaccination and Natural Human Infections. J Immunol. 2017; 198(10):4025-35. Epub 2017/04/05. doi: 10.4049/jimmunol.1700029. PubMed PMID: 28381638; PMCID: PMC5603231.
29. Chuang Y C, Lei H Y, Lin Y S, Liu H S, Wu H L, Yeh T M. Dengue virus-induced autoantibodies bind to plasminogen and enhance its activation. J Immunol. 2011; 187(12):6483-90. Epub 2011/11/11. doi: 10.4049/jimmunol.1102218. PubMed PMID: 22079981.
30. Falgout B, Bray M, Schlesinger J J, Lai C J. Immunization of mice with recombinant vaccinia virus expressing authentic dengue virus nonstructural protein NS1 protects against lethal dengue virus encephalitis. J Virol. 1990; 64(9):4356-63. PubMed PMID: 2143542; PMCID: PMC247903.
31. Srivastava A K, Putnak J R, Warren R L, Hoke C H. Mice immunized with a dengue type 2 virus E and NS1 fusion protein made in Escherichia coli are protected against lethal dengue virus infection. Vaccine. 1995; 13(13):1251-8. PubMed PMID: 8578812.
32. Amorim J H, Diniz M O, Cariri F A, Rodrigues J F, Bizerra R S, Gonçalves AJ, de Barcelos Alves A M, de Souza Ferreira L C. Protective immunity to DENV2 after immunization with a recombinant NS1 protein using a genetically detoxified heat-labile toxin as an adjuvant. Vaccine. 2012; 30(5):837-45. Epub 2011/12/15. doi: 10.1016/j.vaccine.2011.12.034. PubMed PMID: 22178517.
33. Wan S W, Lu Y T, Huang C H, Lin C F, Anderson R, Liu H S, Yeh T M, Yen Y T, Wu-Hsieh B A, Lin Y S. Protection against dengue virus infection in mice by administration of antibodies against modified nonstructural protein 1. PLoS One. 2014; 9(3):e92495. Epub 2014/03/21. doi: 10.1371/journal.pone.0092495. PubMed PMID: 24658118; PMCID: PMC3962419.
34. Costa S M, Freire M S, Alves A M. DNA vaccine against the non-structural 1 protein (NS1) of dengue 2 virus. Vaccine. 2006; 24(21):4562-4. Epub 2005/08/19. doi: 10.1016/j.vaccine.2005.08.022. PubMed PMID: 16140430.
35. Costa S M, Paes M V, Barreto D F, Pinhao A T, Barth O M, Queiroz J L, Arm6a GR, Freire M S, Alves A M. Protection against dengue type 2 virus induced in mice immunized with a DNA plasmid encoding the non-structural 1 (NS1) gene fused to the tissue plasminogen activator signal sequence. Vaccine. 2006; 24(2):195-205. Epub 2005/08/08. doi: 10.1016/j.vaccine.2005.07.059. PubMed PMID: 16122850.
36. Costa S M, Azevedo A S, Paes M V, Sarges F S, Freire M S, Alves A M. DNA vaccines against dengue virus based on the ns1 gene: the influence of different signal sequences on the protein expression and its correlation to the immune response elicited in mice. Virology. 2007; 358(2):413-23. Epub 2006/10/03. doi: 10.1016/j.virol.2006.08.052. PubMed PMID: 17020777.
37. Gonçalves AJ, Oliveira E R, Costa S M, Paes M V, Silva J F, Azevedo A S, Mantuano-Barradas M, Nogueira A C, Almeida C J, Alves A M. Cooperation between CD4+ T Cells and Humoral Immunity Is Critical for Protection against Dengue Using a DNA Vaccine Based on the NS1 Antigen. PLoS Negl Trop Dis. 2015; 9(12):e0004277. Epub 2015/12/09. doi: 10.1371/joumal.pntd.0004277. PubMed PMID: 26650916; PMCID: PMC4674122.
38. Wu S F, Liao C L, Lin Y L, Yeh C T, Chen L K, Huang Y F, Chou H Y, Huang J L, Shaio M F, Sytwu H K. Evaluation of protective efficacy and immune mechanisms of using a non-structural protein NS1 in DNA vaccine against dengue 2 virus in mice. Vaccine. 2003; 21(25-26):3919-29. PubMed PMID: 12922127.
39. Mohsen M O, Zha L, Cabral-Miranda G, Bachmann M F. Major findings and recent advances in virus-like particle (VLP)-based vaccines. Semin Immunol. 2017; 34:123-32. Epub 2017/09/05. doi: 10.1016/j.smim.2017.08.014. PubMed PMID: 28887001.
40. Farlow M R, Andreasen N, Riviere M E, Vostiar I, Vitaliti A, Sovago J, Caputo A, Winblad B, Graf A. Long-term treatment with active AR immunotherapy with CAD106 in mild Alzheimer's disease. Alzheimers Res Ther. 2015; 7(1):23. Epub 2015/04/27. doi: 10.1186/s13195-015-0108-3. PubMed PMID: 25918556; PMCID: PMC4410460.
41. Kündig T M, Senti G, Schnetzler G, Wolf C, Prinz Vavricka B M, Fulurija A, Hennecke F, Sladko K, Jennings G T, Bachmann M F. Der p 1 peptide on virus-like particles is safe and highly immunogenic in healthy adults. J Allergy Clin Immunol. 2006; 117(6):1470-6. Epub 2006/04/27. doi: 10.1016/j.jaci.2006.01.040. PubMed PMID: 16751015.
42. Tissot A C, Maurer P, Nussberger J, Sabat R, Pfister T, Ignatenko S, Volk H D, Stocker H, Muller P, Jennings G T, Wagner F, Bachmann M F. Effect of immunisation against angiotensin II with CYT006-AngQb on ambulatory blood pressure: a double-blind, randomised, placebo-controlled phase IIa study. Lancet. 2008; 371(9615):821-7. Epub 2008/03/11. doi: 10.1016/s0140-6736(08)60381-5. PubMed PMID: 18328929.
43. Ambühl P M, Tissot A C, Fulurija A, Maurer P, Nussberger J, Sabat R, Nief V, Schellekens C, Sladko K, Roubicek K, Pfister T, Rettenbacher M, Volk H D, Wagner F, Müller P, Jennings G T, Bachmann M F. A vaccine for hypertension based on virus-like particles: preclinical efficacy and phase I safety and immunogenicity. J Hypertens. 2007; 25(1):63-72. doi: 10.1097/HJH.0b013e32800ff5d6. PubMed PMID: 17143175.
44. Maurer P, Jennings G T, Willers J, Rohner F, Lindman Y, Roubicek K, Renner W A, Müller P, Bachmann M F. A therapeutic vaccine for nicotine dependence: preclinical efficacy, and Phase I safety and immunogenicity. Eur J Immunol. 2005; 35(7):2031-40. doi: 10.1002/eji.200526285. PubMed PMID: 15971275.
45. Peabody J, Muttil P, Chackerian B, Tumban E. Characterization of a spray-dried candidate HPV L2-VLP vaccine stored for multiple years at room temperature. Papillomavirus Res. 2017; 3:116-20. Epub 2017/04/08. doi: 10.1016/j.pvr.2017.03.004. PubMed PMID: 28720444; PMCID: PMC5604873.
46. Frietze K M, Pascale J M, Moreno B, Chackerian B, Peabody D S. Pathogen-specific deep sequence-coupled biopanning: A method for surveying human antibody responses. PLoS One. 2017; 12(2):e0171511. Epub 2017/02/02. doi: 10.1371/joumal.pone.0171511. PubMed PMID: 28152075; PMCID: PMC5289605.
47. Puerta-Guardo H, Glasner D R, Harris E. Dengue Virus NS1 Disrupts the Endothelial Glycocalyx, Leading to Hyperpermeability. PLoS Pathog. 2016; 12(7):e1005738. Epub 2016/07/14. doi: 10.1371/joumal.ppat.1005738. PubMed PMID: 27416066; PMCID: PMC4944995.
48. Chen C L, Lin C F, Wan S W, Wei L S, Chen M C, Yeh T M, Liu H S, Anderson R, Lin Y S. Anti-dengue virus nonstructural protein 1 antibodies cause NO-mediated endothelial cell apoptosis via ceramide-regulated glycogen synthase kinase-30 and NF-κB activation. J Immunol. 2013; 191(4):1744-52. Epub 2013/07/12. doi: 10.4049/jimmunol.1201976. PubMed PMID: 23851680.
49. Cheng H J, Luo Y H, Wan S W, Lin C F, Wang S T, Hung N T, Liu C C, Ho T S, Liu H S, Yeh T M, Lin Y S. Correlation between serum levels of anti-endothelial cell autoantigen and anti-dengue virus nonstructural protein 1 antibodies in dengue patients. Am J Trop Med Hyg. 2015; 92(5):989-95. Epub 2015/03/09. doi: 10.4269/ajtmh.14-0162. PubMed PMID: 25758647; PMCID: PMC4426591.
50. Singh S, Anupriya M G, Modak A, Sreekumar E. Dengue virus or NS1 protein induces trans-endothelial cell permeability associated with VE-Cadherin and RhoA phosphorylation in HMEC-1 cells preventable by Angiopoietin-1. J Gen Virol. 2018; 99(12):1658-70. Epub 2018/10/24. doi: 10.1099/jgv.0.001163. PubMed PMID: 30355397.

### Second Set of References

1. Messina, J. P.; Brady, O. J.; Golding, N.; Kraemer, M. U. G.; Wint, G. R. W.; Ray, S. E.; Pigott, D. M.; Shearer, F. M.; Johnson, K.; Earl, L.; et al. The Current and Future Global Distribution and Population at Risk of Dengue. Nat. Microbiol. 2019, 4, 1508-1515, doi:10.1038/s41564-019-0476-8.
2. Bhatt, S.; Gething, P. W.; Brady, O. J.; Messina, J. P.; Farlow, A. W.; Moyes, C. L.; Drake, J. M.; Brownstein, J. S.; Hoen, A. G.; Sankoh, O.; et al. The Global Distribution and Burden of Dengue. Nature 2013, 496, 504-507, doi:10.1038/naturel2060.
3. Stanaway, J. D.; Shepard, D. S.; Undurraga, E. A.; Halasa, Y. A.; Coffeng, L. E.; Brady, O. J.; Hay, S. I.; Bedi, N.; Bensenor, I. M.; Castañeda-Orjuela, C. A.; et al. The Global Burden of Dengue: An Analysis from the Global Burden of Disease Study 2013. Lancet Infect. Dis. 2016, 16, 712-723, doi:10.1016/51473-3099(16)00026-8.
4. Halstead, S. B. Safety Issues from a Phase 3 Clinical Trial of a Live-Attenuated Chimeric Yellow Fever Tetravalent Dengue Vaccine. Hum. Vaccine Immunother. 2018, 14, 2158-2162, doi:10.1080/21645515.2018.1445448.
5. Wahala, W. M. P. B.; de Silva, A. M. The Human Antibody Response to Dengue Virus Infection. Viruses 2011, 3, 2374-2395, doi:10.3390/v3122374.
6. Guzman, M. G.; Harris, E. Dengue. Lancet 2015, 385, 453-465, doi:10.1016/50140-6736(14)60572-9.
7. Smith, G. W.; Wright, P. J. Synthesis of Proteins and Glycoproteins in Dengue Type 2 Virus-Infected Vero and Aedes Albopictus Cells. J. Gen. Virol. 1985, 66Pt 3, 559-571, doi:10.1099/0022-1317-66-3-559.
8. Westaway, E. G.; Goodman, M. R. Variation in Distribution of the Three Flavivirus-Specified Glycoproteins Detected by Immunofluorescence in Infected Vero Cells. Arch. Virol. 1987, 94, 215-228, doi:10.1007/BF01310715.
9. Winkler, G.; Randolph, V. B.; Cleaves, G. R.; Ryan, T. E.; Stollar, V. Evidence That the Mature Form of the Flavivirus Nonstructural Protein N S1 Is a Dimer. Virology 1988, 162, 187-196, doi:10.1016/0042-6822(88)90408-4.
10. Mason, P. W. Maturation of Japanese Encephalitis Virus Glycoproteins Produced by Infected Mammalian and Mosquito Cells. Virology 1989, 169, 354-364, doi:10.1016/0042-6822(89)90161-X.
11. Gutsche, I.; Coulibaly, F.; Voss, J. E.; Salmon, J.; d'Alayer, J.; Ermonval, M.; Larquet, E.; Charneau, P.; Krey, T.; Megret, F.; et al. Secreted Dengue Virus Nonstructural Protein N S1 Is an Atypical Barrel-Shaped High-Density Lipoprotein. Proc. Natl. Acad. Sci. USA 2011, 108, 8003-8008, doi:10.1073/pnas.1017338108.
12. Beatty, P. R.; Puerta-Guardo, H.; Killingbeck, S. S.; Glasner, DR.; Hopkins, K.; Harris, E. Dengue Virus NS1 Triggers Endothelial Permeability and Vascular Leak That Is Prevented by NS1 Vaccination. Sci. Transl. Med. 2015, 7, 304ra141, doi:10.1126/scitranslmed.aaa3787.
13. Modhiran, N.; Watterson, D.; Muller, D. A.; Panetta, A. K.; Sester, D. P.; Liu, L.; Hume, D. A.; Stacey, K. J.; Young, P. R. Dengue Virus NS1 Protein Activates Cells via Toll-like Receptor 4 and Disrupts Endothelial Cell Monolayer Integrity. Sci. Transl. Med. 2015, 7, 304ra142, doi:10.1126/scitranslmed.aaa3863.
14. Glasner, D. R.; Ratnasiri, K.; Puerta-Guardo, H.; Espinosa, D. A.; Beatty, P. R.; Harris, E. Dengue Virus NS1 Cytokine-Independent Vascular Leak Is Dependent on Endothelial Glycocalyx Components. PloS Pathog. 2017, 13, e1006673, doi:10.1371/journal.ppat.1006673.
15. Chen, H. R.; Chuang, Y. C.; Lin, Y. S.; Liu, H. S.; Liu, C. C.; Pemg, G. C.; Yeh, T. M. Dengue Virus Nonstructural Protein 1 Induces Vascular Leakage through Macrophage Migration Inhibitory Factor and Autophagy. PloS Negl. Trop. Dis. 2016, 10, e0004828, doi:10.1371/journal. pntd.0004828.
16. Puerta-Guardo, H.; Glasner, D. R.; Harris, E. Dengue Virus NS1 Disrupts the Endothelial Glycocalyx, Leading to Hyperpermeability. PloS Pathog. 2016, 12, e1005738, doi:10.1371/joumal.ppat.1005738.
17. Wan, S. W.; Lu, Y. T.; Huang, C. H.; Lin, C. F.; Anderson, R.; Liu, H. S.; Yeh, T. M.; Yen, Y. T.; Wu-Hsieh, B. A.; Lin, Y. S. Protection against Dengue Virus Infection in Mice by Administration of Antibodies against Modified Nonstructural Protein 1. PloS ONE 2014, 9, e92495, doi:10.1371/joumal.pone.0092495.
18. Lai, Y. C.; Chuang, Y. C.; Liu, C. C.; Ho, T. S.; Lin, Y. S.; Anderson, R.; Yeh, T. M. Antibodies Against Modified NS1 Wing Domain Peptide Protect Against Dengue Virus Infection. Sci. Rep. 2017, 7, 6975, doi:10.1038/s41598-017-07308-3.
19. Wan, S. W.; Chen, P. W.; Chen, C. Y.; Lai, Y. C.; Chu, Y. T.; Hung, C. Y.; Lee, H.; Wu, H. F.; Chuang, Y. C.; Lin, J.; et al. Therapeutic Effects of Monoclonal Antibody against Dengue Virus NS1 in a STAT1 Knockout Mouse Model of Dengue Infection. J Immunol. 2017, 199, 2834-2844, doi:10.4049/jimmunol.1601523.
20. Chu, Y. T.; Wan, S. W.; Chang, Y. C.; Lee, C. K.; Wu-Hsieh, B. A.; Anderson, R.; Lin, Y. S. Antibodies against Nonstructural Protein 1 Protect Mice from Dengue Virus-Induced Mast Cell Activation. Lab. Investig. 2017, 97, 602-614, doi:10.1038/labinvest.2017.10.
21. Falgout, B.; Bray, M.; Schlesinger, J. J. Against Lethal Dengue Virus Encephalitis. J. Virol. 1990, 64, 8.
22. Amorim, J. H.; Diniz, M. O.; Cariri, F. A. M. O.; Rodrigues, J. F.; Bizerra, R. S. P.; Gonçalves, A. J. S.; de Barcelos Alves, A. M.; de Souza Ferreira, L. C. Protective Immunity to DENV2 after Immunization with a Recombinant NS1 Protein Using a Genetically Detoxified Heat-Labile Toxin as an Adjuvant. Vaccine 2012, 30, 837-845, doi:10.1016/j.vaccine.2011.12.034.
23. Wu, S. F.; Liao, C. L.; Lin, Y. L.; Yeh, C. T.; Chen, L. K.; Huang, Y. F.; Chou, H. Y.; Huang, J. L.; Shaio, M. F.; Sytwu, H. K. Evaluation of Protective Efficacy and Immune Mechanisms of Using a Non-Structural Protein N S1 in DNA Vaccine against Dengue 2 Virus in Mice. Vaccine 2003, 21, 3919-3929, doi:10.1016/50264-410X(03)00310-4.
24. Costa, S. M.; Azevedo, A. S.; Paes, M. V.; Sarges, F. S.; Freire, M. S.; Alves, A. M. B. DNA Vaccines against Dengue Virus Based on the Nsl Gene: The Influence of Different Signal Sequences on the Protein Expression and Its Correlation to the Immune Response Elicited in Mice. Virology 2007, 358, 413-423, doi:10.1016/j.virol.2006.08.052.
25. Gonçalves, A. J. S.; Oliveira, E. R. A.; Costa, S. M.; Paes, M. V.; Silva, J. F. A.; Azevedo, A. S.; Mantuano-Barradas, M.; Nogueira, A. C. M. A.; Almeida, C. J.; Alves, A. M. B. Cooperation between CD4+ T Cells and Humoral Immunity Is Critical for Protection against Dengue Using a DNA Vaccine Based on the NS1 Antigen. PloS Negl. Trop. Dis. 2015, 9, e0004277, doi:10.1371/journal. pntd.0004277.
26. Costa, S. M.; Freire, M. S.; Alves, A. M. B. DNA Vaccine against the Non-Structural 1 Protein (NS1) of Dengue 2 Virus. Vaccine 2006, 24, 4562-4564, doi:10.1016/j.vaccine.2005.08.022.
27. Schlesinger, J. J.; Brandriss, M. W.; Walsh, E. E. Protection of Mice against Dengue 2 Virus Encephalitis by Immunization with the Dengue 2 Virus Non-Structural Glycoprotein NS1. J Gen. Virol. 1987, 68 Pt 3, 853-857, doi:10.1099/0022-1317-68-3-853.
28. Srivastava, A. K.; Putnak, J. R.; Warren, R. L.; Hoke, C. H. Mice Immunized with a Dengue Type 2 Virus E and NS1 Fusion Protein Made in Escherichia Coli Are Protected against Lethal Dengue Virus Infection. Vaccine 1995, 13, 1251-1258, doi:10.1016/0264-410x(94)00059-v.
29. Chen, H. R.; Lai, Y. C.; Yeh, T. M. Dengue Virus Non-Structural Protein 1: A Pathogenic Factor, Therapeutic Target, and Vaccine Candidate. J. Biomed. Sci. 2018, 25, 58, doi:10.1186/s12929-018-0462-0.
30. Donaldson, B.; Lateef, Z.; Walker, G. F.; Young, S. L.; Ward, V. K. Virus-like Particle Vaccines: Immunology and Formulation for Clinical Translation. Expert Rev. Vaccines 2018, 17, 833-849, doi:10.1080/14760584.2018.1516552.
31. Jelínková, L.; Jhun, H.; Eaton, A.; Petrovsky, N.; Zavala, F.; Chackerian, B. An Epitope-Based Malaria Vaccine Targeting the Junctional Region of Circumsporozoite Protein. NPJ Vaccines 2021, 6, 13, doi:10.1038/s41541-020-00274-4.
32. Mogus, A. T.; Liu, L.; Jia, M.; Ajayi, D. T.; Xu, K.; Kong, R.; Huang, J.; Yu, J.; Kwong, P. D.; Mascola, J. R.; et al. Virus-Like Particle Based Vaccines Elicit Neutralizing Antibodies against the HIV-1 Fusion Peptide. Vaccines 2020, 8, 765, doi:10.3390/vaccines8040765.
33. Crossey, E.; Amar, M. J. A.; Sampson, M.; Peabody, J.; Schiller, J. T.; Chackerian, B.; Remaley, A. T. A Cholesterol-Lowering VLP Vaccine That Targets PCSK9. Vaccine 2015, 33, 5747-5755, doi:10.1016/j.vaccine.2015.09.044.
34. Maphis, N. M.; Peabody, J.; Crossey, E.; Jiang, S.; Jamaleddin Ahmad, F. A.; Alvarez, M.; Mansoor, S. K.; Yaney, A.; Yang, Y.; Sillerud, L. O.; et al. Qβ Virus-like Particle-Based Vaccine Induces Robust Immunity and Protects against Tauopathy. NPJ Vaccines 2019, 4, doi:10.1038/s41541-019-0118-4.
35. Caldeira, J. d. C.; Medford, A.; Kines, R. C.; Lino, C. A.; Schiller, J. T.; Chackerian, B.; Peabody, D. S. Immunogenic Display of Diverse Peptides, Including a Broadly Cross-Type Neutralizing Human Papillomavirus L2 Epitope, on Virus-like Particles of the RNA Bacteriophage PP7. Vaccine 2010, 28, 4384-4393, doi:10.1016/j.vaccine.2010.04.049.
36. Tumban, E.; Peabody, J.; Peabody, D. S.; Chackerian, B. A Pan-HPV Vaccine Based on Bacteriophage PP7 VLPs Displaying Broadly Cross-Neutralizing Epitopes from the HPV Minor Capsid Protein, L2. PloS ONE 2011, 6, doi:10.1371/journal.pone.0023310.
37. Frietze, K. M.; Pascale, J. M.; Moreno, B.; Chackerian, B.; Peabody, D. S. Pathogen-Specific Deep Sequence-Coupled Biopanning: A Method for Surveying Human Antibody Responses. PloS ONE 2017, 12, e0171511, doi:10.1371/journal.pone.0171511.
38. Hertz, T.; Beatty, P. R.; MacMillen, Z.; Killingbeck, S. S.; Wang, C.; Harris, E. Antibody Epitopes Identified in Critical Regions of Dengue Virus Nonstructural 1 Protein in Mouse Vaccination and Natural Human Infections. J. Immunol. 2017, 198, 4025-4035, doi:10.4049/jimmunol.1700029.
39. Falconar, A. K. The Dengue Virus Nonstructural-1 Protein (NS1) Generates Antibodies to Common Epitopes on Human Blood Clotting, Integrin/Adhesin Proteins and Binds to Human Endothelial Cells: Potential Implications in Haemorrhagic Fever Pathogenesis. Arch. Virol. 1997, 142, 897-916, doi:10.1007/s007050050127.
40. Falconar, A. K. I. Antibody Responses Are Generated to Immunodominant ELK/KLE-Type Motifs on the Nonstructural-1 Glycoprotein during Live Dengue Virus Infections in Mice and Humans: Implications for Diagnosis, Pathogenesis, and Vaccine Design. Clin. Vaccine Immunol. 2007, 14, 493-504, doi:10.1128/CVI.00371-06.
41. Lin, C. F.; Wan, S. W.; Chen, M. C.; Lin, S. C.; Cheng, C. C.; Chiu, S. C.; Hsiao, Y. L.; Lei, H. Y.; Liu, H. S.; Yeh, T. M.; et al. Liver Injury Caused by Antibodies against Dengue Virus Nonstructural Protein 1 in a Murine Model. Lab. Investig. 2008, 88, 1079-1089, doi:10.1038/labinvest.2008.70.
42. Lin, Y. S.; Yeh, T. M.; Lin, C. F.; Wan, S. W.; Chuang, Y. C.; Hsu, T. K.; Liu, H. S.; Liu, C. C.; Anderson, R.; Lei, H. Y. Molecular Mimicry between Virus and Host and Its Implications for Dengue Disease Pathogenesis. Exp. Biol. Med. 2011, 236, 515-523, doi:10.1258/ebm.2011.010339.
43. Chuang, Y. C.; Wang, S. Y.; Lin, Y. S.; Chen, H. R.; Yeh, T. M. Re-Evaluation of the Pathogenic Roles of Nonstructural Protein 1 and Its Antibodies during Dengue Virus Infection. J. Biomed. Sci. 2013, 20, 42, doi:10.1186/1423-0127-20-42.
44. Chuang, Y. C.; Lei, H. Y.; Liu, H. S.; Lin, Y. S.; Fu, T. F.; Yeh, T. M. Macrophage Migration Inhibitory Factor Induced by Dengue Virus Infection Increases Vascular Permeability. Cytokine 2011, 54, 222-231, doi:10.1016/j.cyto.2011.01.013.
45. Sun, D. S.; King, C. C.; Huang, H. S.; Shih, Y. L.; Lee, C. C.; Tsai, W. J.; Yu, C. C.; Chang, H. H. Antiplatelet Autoantibodies Elicited by Dengue Virus Non-Structural Protein 1 Cause Thrombocytopenia and Mortality in Mice. J. Thromb. Haemost. 2007, 5, 2291-2299, doi:10.1111/j.1538-7836.2007.02754.x.
46. Cheng, H. J.; Lei, H. Y.; Lin, C. F.; Luo, Y. H.; Wan, S. W.; Liu, H. S.; Yeh, T. M.; Lin, Y. S. Anti-Dengue Virus Nonstructural Protein 1 Antibodies Recognize Protein Disulfide Isomerase on Platelets and Inhibit Platelet Aggregation. Mol. Immunol. 2009, 47, 398-406, doi:10.1016/j.molimm.2009.08.033.
47. Chen, M. C.; Lin, C. F.; Lei, H. Y.; Lin, S. C.; Liu, H. S.; Yeh, T. M.; Anderson, R.; Lin, Y. S. Deletion of the C-Terminal Region of Dengue Virus Nonstructural Protein 1 (NS1) Abolishes Anti-NS1-Mediated Platelet Dysfunction and Bleeding Tendency. J. Immunol. 2009, 183, 1797-1803, doi:10.4049/jimmunol.0800672.
48. Liu, I. J.; Chiu, C. Y.; Chen, Y. C.; Wu, H. C. Molecular Mimicry of Human Endothelial Cell Antigen by Autoantibodies to Nonstructural Protein 1 of Dengue Virus. J. Biol. Chem. 2011, 286, 9726-9736, doi:10.1074/jbc.M110.170993.
49. Chao, C. H.; Wu, W. C.; Lai, Y. C.; Tsai, P. J.; Pemg, G. C.; Lin, Y. S.; Yeh, T. M. Dengue Virus Nonstructural Protein 1 Activates Platelets via Toll-like Receptor 4, Leading to Thrombocytopenia and Hemorrhage. PloS Pathog. 2019, 15, e1007625, doi:10.1371/journal.ppat.1007625.
50. Warner, N. L.; Linville, A. C.; Core, S. B.; Moreno, B.; Pascale, J. M.; Peabody, D. S.; Chackerian, B.; Frietze, K. M. Expansion and Refinement of Deep Sequence-Coupled Biopanning Technology for Epitope-Specific Antibody Responses in Human Serum. Viruses 2020, 12, 1114, doi:10.3390/v12101114.
51. Akey, D. L.; Brown, W. C.; Dutta, S.; Konwerski, J.; Jose, J.; Jurkiw, T. J.; DelProposto, J.; Ogata, C. M.; Skiniotis, G.; Kuhn, R. J.; et al. Flavivirus NS1 Structures Reveal Surfaces for Associations with Membranes and the Immune System. Science 2014, 343, 881-885, doi:10.1126/science.1247749.
52. Henchal, E. A.; Henchal, L. S.; Schlesinger, J. J. Synergistic Interactions of Anti-NS1 Monoclonal Antibodies Protect Passively Immunized Mice from Lethal Challenge with Dengue 2 Virus. J. Gen. Virol. 1988, 69 Pt 8, 2101-2107, doi:10.1099/0022-1317-69-8-2101.
53. Lai, C. Y.; Tsai, W. Y.; Lin, S. R.; Kao, C. L.; Hu, H. P.; King, C. C.; Wu, H. C.; Chang, G. J.; Wang, W. K. Antibodies to Envelope Glycoprotein of Dengue Virus during the Natural Course of Infection Are Predominantly Cross-Reactive and Recognize Epitopes Containing Highly Conserved Residues at the Fusion Loop of Domain II. J. Virol. 2008, 82, 6631-6643, doi:10.1128/JVI.00316-08.
54. Crill, W. D.; Hughes, H. R.; Delorey, M. J.; Chang, G. J. J. Humoral Immune Responses of Dengue Fever Patients Using Epitope-Specific Serotype-2 Virus-like Particle Antigens. PloS ONE 2009, 4, e4991, doi:10.1371/journal.pone.0004991.
55. Wahala, W. M. P. B.; Kraus, A. A.; Haymore, L. B.; Accavitti-Loper, M. A.; de Silva, A. M.

Dengue Virus Neutralization by Human Immune Sera: Role of Envelope Protein Domain III-Reactive Antibody. Virology 2009, 392, 103-113, doi:10.1016/j.virol.2009.06.037.
56. Lin, H. E.; Tsai, W. Y.; Liu, I. J.; Li, P. C.; Liao, M. Y.; Tsai, J. J.; Wu, Y. C.; Lai, C. Y.; Lu, C. H.; Huang, J. H.; et al. Analysis of Epitopes on Dengue Virus Envelope Protein Recognized by Monoclonal Antibodies and Polyclonal Human Sera by a High Throughput Assay. PloS Negl. Trop. Dis. 2012, 6, e1447, doi:10.1371/journal. pntd.0001447.
57. Lai, C. Y.; Williams, K. L.; Wu, Y. C.; Knight, S.; Balmaseda, A.; Harris, E.; Wang, W. K. Analysis of Cross-Reactive Antibodies Recognizing the Fusion Loop of Envelope Protein and Correlation with Neutralizing Antibody Titers in Nicaraguan Dengue Cases. PloS Negl. Trop. Dis. 2013, 7, e2451, doi:10.1371/joumal.pntd.0002451.
58. Wang, C.; Puerta-Guardo, H.; Biering, S. B.; Glasner, D. R.; Tran, E. B.; Patana, M.; Gomberg, T. A.; Malvar, C.; Lo, N. T. N.; Espinosa, D. A.; et al. Endocytosis of Flavivirus NS1 Is Required for NS1-Mediated Endothelial Hyperpermeability and Is Abolished by a Single N-Glycosylation Site Mutation. PloS Pathog. 2019, 15, e1007938, doi:10.1371/joumal.ppat.1007938.
59. Frietze, K. M.; Peabody, D. S.; Chackerian, B. Engineering Virus-like Particles as Vaccine Platforms. Curr. Opin. Virol. 2016, 18, 44-49, doi:10.1016/j.coviro.2016.03.001.
60. O'Rourke, J. P.; Peabody, D. S.; Chackerian, B. Affinity Selection of Epitope-Based Vaccines Using a Bacteriophage Virus-like Particle Platform. Curr. Opin. Virol. 2015, 11, 76-82, doi:10.1016/j.coviro.2015.03.005.

SEQUENCES

| Disclosed Sequences | |
|---|---|
| ImmunoPeptides | SEQ ID NO: |
| VHTWTEQYFKQ | SEQ ID NO: 1; |
| GIRSTTRLENVMWKQI | SEQ ID NO: 2; |
| EVEDYGFGVFTTNIWLK | SEQ ID NO: 3; |
| RLMSAAIKD | SEQ ID NO: 4; |
| AVHADMGYWIES | SEQ ID NO: 5; |
| WPKSHTLWSNGVLES | SEQ ID NO: 6; |
| RGPSLRTTTVSGK | SEQ ID NO: 7; |
| GEDGCWYGMEIRP | SEQ ID NO: 8; |
| GPWHLGKLELDF | SEQ ID NO: 9; |
| KYSWSEWGAK | SEQ ID NO: 10; |
| DSGCVVSWKNKELKC | SEQ ID NO: 14; |
| ITDNVHTWTEQYKFQ | SEQ ID NO: 15; |
| TRLENLMWKQITPEL | SEQ ID NO: 16; |
| ITPELNHILSENEVK | SEQ ID NO: 17; |
| AECPNTNRAWNSLEV | SEQ ID NO: 18; |
| MIIPKNLAGPVSQHN | SEQ ID NO: 19; |
| VSQHNYRPGYHTQIT | SEQ ID NO: 20; |
| TTASGKLITEWCCRS | SEQ ID NO: 21; |
| LRYRGEDGCWYGMEI | SEQ ID NO: 22; |
| KELKCGSGIFITDNV | SEQ ID NO: 23; |
| QYKFQPESPSKLASA | SEQ ID NO: 24; |
| KLASAIQKAHEEGIC | SEQ ID NO: 25; |
| EEGICGIRSVTRLEN | SEQ ID NO: 26; |
| ENEVKLTIMTGDIKG | SEQ ID NO: 27; |
| GDIKGIMQAGKRSLR | SEQ ID NO: 28; |
| KRSLRPQPTELKYSW | SEQ ID NO: 29; |
| LKYSWKTWGKAKMLS | SEQ ID NO: 30; |
| AKMLSTESHNQTFLI | SEQ ID NO: 31; |
| QTFLIDGPETAECPN | SEQ ID NO: 32; |
| NSLEVEDYGFGVFTT | SEQ ID NO: 33; |
| GVFTTNIWLKLKEKQ | SEQ ID NO: 34; |
| LKEKQDVFCDSKLMS | SEQ ID NO: 35; |
| SKLMSAAIKDNRAVH | SEQ ID NO: 36; |
| NRAVHADMGYWIESA | SEQ ID NO: 37; |
| WIESALNDTWKIEKA | SEQ ID NO: 38; |
| KIEKASFIEVKNCHW | SEQ ID NO: 39; |
| KNCHWPKSHTLWSNG | SEQ ID NO: 40; |
| LWSNGVLESEMIIPK | SEQ ID NO: 41; |
| HTQITGPWHLGKLEM | SEQ ID NO: 42; |
| GKLEMDFDFCDGTTV | SEQ ID NO: 43; |
| DGTTVVVTEDCGNRG | SEQ ID NO: 44; |
| CGNRGPSLRTTTASG | SEQ ID NO: 45; |
| WCCRSCTLPPLRYRG | SEQ ID NO: 46; |
| YGMEIRPLKEKEENL | SEQ ID NO: 47; |
| LKEKEENLVNSLVTA | SEQ ID NO: 48. |

1. A composition comprising: (a) a virus-like particle (VLP) comprising a bacteriophage coat protein; and (b) at least one immunogenic peptide; wherein said immunogenic peptide is conjugated onto said virus-like particle, and wherein said immunogenic peptide is between 5 and 17 contiguous amino acids derived from a conserved or non-conserved region of NS1 peptide of dengue virus (DENV).

2. The composition of claim 1, wherein said immunogenic peptide is conjugated to an amino acid residue on said bacteriophage coat protein of said VLP.

3. The composition according to claim 1 wherein said immunogenic peptide is conjugated to a lysine residue.

4. The composition according to claim 1, wherein said immunogenic peptide is displayed on the surface of said VLP.

5. The composition according to claim 1 wherein the bacteriophage coat protein is a coat protein derived from Qbeta or AP205 bacteriophage.

6. The composition according to claim 1 wherein said bacteriophage coat protein is a dimer coat protein.

7. The composition according to claim 1 wherein said bacteriophage coat protein is a coat protein derived from Qbeta bacteriophage.

8. The composition according to claim 1 wherein said bacteriophage coat protein is a coat protein derived from AP205 bacteriophage.

9. The composition according to claim 1 wherein said immunogenic peptide is conjugated to the aminobutylene side chain of a lysine residue on the surface of the bacteriophage.

10. The composition according to claim 9 wherein said immunogenic peptide is conjugated onto said VLP at said lysine residue by covalently binding said immunogenic peptide to said lysine residue through a linker group.

11. The composition according to claim 10 wherein said linker group comprises a crosslinker and an oligopeptide.

12. (canceled)

13. (canceled)

14. (canceled)

15. (canceled)

16. The composition according to claim 11 wherein said oligopeptide is a 3 to 15 mer oligopeptide comprising neutral amino acid residues, said oligopeptide being bonded directly to said immunogenic peptide.

17. (canceled)

18. (canceled)

19. (canceled)

20. The composition according to claim 16 wherein said oligopeptide comprises at least one cysteinyl group.

21. The composition according to claim 20 wherein said oligopeptide comprises a cysteinyl group at the carboxyl end which is bonded directly to a crosslinker through a sulfur group.

22. The composition according to claim 21 wherein said crosslinker is (Succininidyl-6-[β-maleimidopropionanido]hexanoate) (SMPH).

23. The composition according to claim 16 wherein said oligopeptide is bonded directly to the carboxyl end of said immunogenic peptide.

24. (canceled)

25. (canceled)

26. The composition according to claim 1 wherein said immunogenic peptide is an amino acid sequence comprising at least 5 contiguous amino acid residues of any one of the amino acid sequences of SEQ ID Nos: 1-8.

27. The composition according to claim 1 wherein said immunogenic peptide is an amino acid sequence of SEQ ID Nos:1-8 and 10.

28. The composition according to claim 1 wherein said immunogenic peptide is an amino acid sequence of SEQ ID NO: 1-8.

29. The composition according to claim 1 wherein said immunogenic peptide is an amino acid sequence of peptides 1-35 (SEQ ID NOs: 14-48) of FIG. 10A.

30. The composition according to claim 1 wherein said immunogenic peptide is an amino acid sequence of SEQ ID NOs:14-22.

31. A composition comprising: (a) a virus-like particle (VLP) comprising 94180 Qβ bacteriophage single chain coat proteins which self-assemble into said VLP; and (b) at least one immunogenic peptide; wherein said immunogenic peptide is conjugated onto said VLP, wherein said immunogenic peptide is between 5 and 17 contiguous amino acids of amino acid sequences SEQ ID Nos: 1-10 and 14-22 and said immunogenic peptide is conjugated to said VLP through a linker molecule comprising a crosslinker covalently bonded to an oligopeptide, wherein said crosslinker is (Succinimidyl-6-[β-maleimidopropionamido]hexanoate) (SMPH) and said oligopeptide is GGGC of SEQ ID NO: 13 wherein said N-terminal glycine residue of said oligopeptide is covalently bonded to the carboxyl terminus of said immunogenic peptide and said carboxyl terminal cysteinyl residue is covalently bonded to the crosslinker and the crosslinker is also bonded to a lysine residue on the VLP.

32. The composition according to claim 31 wherein said immunogenic peptide is an amino acid sequence according to any one of SEQ ID Nos: 1-8 and 10.

33. The composition according to claim 31 wherein said immunogenic peptide is an amino acid sequence according to any one of SEQ ID Nos: 1-8.

34. The composition according to claim 31 wherein said immunogenic peptide is not the full length amino acid sequence of SEQ ID NO: 9.

35. The composition according to claim 31 wherein said immunogenic peptide is the amino acid sequence of SEQ ID NO: 10.

36. The composition according to claim 31 wherein said immunogenic peptide is an amino sequence of SEQ ID NOs:14-22 or 23-48.

37. A population of virus-like particles according to claim 1.

38. A pharmaceutical composition comprising a population of virus-like particles according to claim 37 in combination with a pharmaceutically acceptable carrier, additive and/or excipient.

39. The composition according to claim 38 which is formulated as a vaccine for administration to a subject or patient.

40. The composition according to claim 38 wherein said composition comprises an adjuvant.

41. (canceled)

42. (canceled)

43. (canceled)

44. (canceled)

45. A method for treating or inhibiting a dengue virus (DENV) infection, morbidity, or a symptom thereof in a patient or subject in need comprising administering to said patient a composition according to claim 38 to said patient or subject.

46. The method of claim 45 wherein said infection is dengue fever.

47. The method of claim 45 wherein said infection is severe dengue.

48. The method according to claim 45 wherein said symptom is one or more of high fever, aches and pains across the body, nausea, vomiting, skin rash, loss of appetite, headache, abdominal pain, bloody gums and nose, blood in stools, blood vessel damage, organ dysfunction in heart, lungs and/or liver, blood in vomit, bruise-like formations on the skin and bleeding under the epidermis.

49-62. (canceled)
